(19) United States
(12) Patent Application Publication
(10) Pub. No.: US 2024/0262838 A1
(43) Pub. Date: Aug. 8, 2024

(54) NITROGEN-CONTAINING HETEROCYCLIC COMPOUNDS AND USES THEREOF

(71) Applicant: GUANGZHOU CHINARAY OPTOELECTRONIC MATERIALS LTD., Guangzhou, Guangdong (CN)

(72) Inventors: Chi Ming LEUNG, Guangzhou, Guangdong (CN); Ruifeng HE, Guangzhou, Guangdong (CN); Yan LI, Guangzhou, Guangdong (CN); Haoran WANG, Guangzhou, Guangdong (CN)

(73) Assignee: GUANGZHOU CHINARAY OPTOELECTRONIC MATERIALS LTD., Guangzhou, Guangdong (CN)

(21) Appl. No.: 18/682,421

(22) PCT Filed: May 19, 2021
(86) PCT No.: PCT/CN2021/094647

§ 371 Date: Feb. 8, 2024

(30) Foreign Application Priority Data
Dec. 17, 2020 (CN) 202011492791.6

(51) Int. Cl. C07D 487/16; C07D 487/22; C07D 519/00

(52) U.S. Cl. CPC C07D 487/16; C07D 487/22; C07D 519/00

(57) ABSTRACT

The present application provides a nitrogen-containing heterocyclic compound and uses thereof. The nitrogen-containing heterocyclic compound has a structure represented by formula (1), in which Ar₁ is selected from a substituted or unsubstituted aromatic group having 6 to 40 ring atoms or a substituted or unsubstituted heteroaromatic group having 5 to 40 ring atoms, X₁ at each occurrence is independently selected from CR₂ or N, X₂ at each occurrence is independently selected from CR₃ or N, X₃ at each occurrence is independently selected from CR₄ or N, and X₄ at each occurrence is independently selected from CR₅ or N.

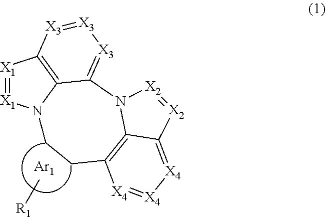

### CROSS-REFERENCE TO RELATED APPLICATIONS

This application claims the priority to and benefit of Chinese Patent Application No. 2020114927916, filed on Dec. 17, 2020, and titled “NITROGEN-CONTAINING HETEROCYCLIC COMPOUNDS AND USES THEREOF”, the disclosure of which is incorporated herein by reference in its entirety.

### TECHNICAL FIELD

The disclosure relates to the field of organic electroluminescence, and in particular, to nitrogen-containing heterocyclic compounds and uses thereof.

### BACKGROUND

Organic optoelectronic materials have advantages of diversity in synthesis, relatively low manufacturing cost, and excellent optical and electrical properties. Organic light-emitting diodes (OLEDs) have broad development potential in the uses of optoelectronic devices (for example, flat panel displays and illumination devices), due to their advantages such as wide viewing angle, fast response time, low operating voltage, thin thicknesses of panels, and the like.

In order to improve luminous efficiency of organic light-emitting diodes, various light-emitting material systems based on fluorescence and phosphorescence have been developed. Organic light-emitting diodes using fluorescent materials have the characteristic of high reliability, but their internal electroluminescence quantum efficiency is 25% or less under electrical excitation. This is because the ratio of the singlet excited state to the triplet excited state of excitons generated by the current is 1:3. In contrast, organic light-emitting diodes using phosphorescent materials have achieved almost 100% internal electroluminescence quantum efficiency, so the development of phosphorescent light-emitting materials has been extensively researched.

In addition to the development of emitters (guest materials) mentioned above, matrix materials (host materials) also play an important role in improving color purity, luminous efficiency, and stability. When using the host materials/the guest materials system as materials of the light-emitting layers, because the host materials have significant impact on efficiency and characteristics of electroluminescent devices, the selection of the host materials is important.

At present, 4,4′-bis(N-carbazolyl)-1,1′-biphenyl (CBP) is the most widely used phosphorescent substance as the host material. In recent years, Pioneer Electronic Corporation (Japan) and others have developed a high performance organic electroluminescent device, which uses compounds such as bis(2-methyl)-8-hydroxyquinoline-4-phenylphenol aluminum (III) (BAlq) and phenanthroline (BCP) as host materials. However, performances and lifetime of the devices obtained using these materials still need to be improved.

Therefore, solutions using the host materials still need to be improved and developed.

### SUMMARY

An object of the disclosure is to provide a nitrogen-containing heterocyclic compound and uses thereof, aiming to provide a new type of host material to improve efficiency and lifetime of devices.

The nitrogen-containing heterocyclic compound has a structure represented by formula (1):

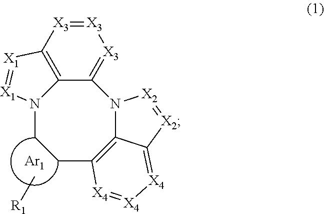

wherein,
Ar₁ is selected from a substituted or unsubstituted aromatic group having 6 to 40 ring atoms or a substituted or unsubstituted heteroaromatic group having 5 to 40 ring atoms;
X₁ at each occurrence is independently selected from CR₂ or N;
X₂ at each occurrence is independently selected from CR₃ or N;
X₃ at each occurrence is independently selected from CR₄ or N;
X₄ at each occurrence is independently selected from CR₅ or N;
R₁ to R₅ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
R₁ and X₄ are connected to form a ring or not form a ring together; two adjacent R₂ groups are connected to form a ring or not form a ring together; two adjacent R₃ groups are connected to form a ring or not form a ring together; two adjacent R₄ groups are connected to form a ring or not form a ring together; and two adjacent R₅ groups are connected to form a ring or not form a ring together.

The disclosure further relates to a mixture that includes the nitrogen-containing heterocyclic compound as described above, and at least one organic functional material selected from a hole injection material, a hole transport material, an electron transport material, an electron injection material, an electron blocking material, a hole blocking material, a light-emitting material, a host material, and/or an organic dye.

The disclosure further relates to a composition that includes the nitrogen-containing heterocyclic compound or the mixture as described above, and at least one organic solvent.

The disclosure further relates to an organic electronic device that includes at least one functional layer. A material of the functional layer includes the nitrogen-containing heterocyclic compound or the mixture as described above, or the functional layer is prepared from the composition as described above.

### Beneficial Effects

The nitrogen-containing heterocyclic compound provided in the disclosure can be used in OLEDs, especially as host materials of light-emitting layers, which can improve external quantum efficiency of devices and prolong their lifetime. At the same time, the compound provided in the disclosure not only has a relatively stable eight-membered ring structure, but also has a larger ring structure, which can increase the conjugation system of the compound, make the compound more stable, thus improving stability of devices and reducing starting voltages to increase lifetime of devices.

### DETAILED DESCRIPTION

In order to make the object, technical solutions, and advantages of the disclosure clearer and more definite, the following contents will provide a detailed description of the disclosure in conjunction with the embodiments. It should be understood that the specific embodiments described here are only used to illustrate the disclosure and not intended to limit the disclosure.

Unless otherwise defined, all technical and scientific terms used in this context have the same meanings as those commonly understood by those skilled in the art. The terms used in the specification of the disclosure are only for the purpose of describing specific embodiments and are not intended to limit the disclosure. The term “and/or” used in this context includes any and all combinations of one or more related listed items.

The disclosure provides a nitrogen-containing heterocyclic compound and uses thereof. In order to make the object, the technical solutions, and effects of the disclosure clearer and more definite, the following will provide a further detailed description of the disclosure. It should be understood that the embodiments described here are only used to explain the disclosure and are not intended to limit the disclosure.

In the disclosure, the term “substituted” means that a hydrogen atom in one substituted group is substituted by a substituent group.

In the disclosure, the same substituent groups at different substituent sites may be independently selected from different groups. For example, if a formula includes multiple R₁ groups, the R₁ groups may be independently selected from different groups.

In the disclosure, the term “substituted or unsubstituted” means that a defined group may be substituted or not be substituted. When the defined group is substituted, it can be understood that the defined group is optionally substituted by acceptable groups in the art, which include, but not limited to, -D, a cyano group, an isocyano group, a nitro group, a halogen atom, a C₁₋₁₀ alkyl group, a C₁₋₁₀ alkoxy group, a C₁₋₁₀ alkyl sulfur group, a C₆₋₃₀ aryl group, a C₆₋₃₀ aryloxy group, a C₆₋₃₀ aryl sulfur group, a C₃₋₃₀ heteroaryl group, a C₁₋₃₀ silyl group, a C₂₋₁₀ alkylamine group, a C₆₋₃₀ arylamine group, or any combination of these groups.

In the disclosure, the term “the number of ring atoms” refers to a number of atoms constituting a ring of a structural compound obtained by atomic bonding, for example, a monocyclic compound, a fused ring compound, a cross-linked compound, a carbon ring compound, or a heterocyclic compound. In a ring substituted by a substituent group, the atoms contained in the substituent group are not included in the atoms forming the ring. The term “the number of ring atoms” is used in the context for the same meaning unless otherwise specified. For example, the number of ring atoms in a benzene ring is 6, the number of ring atoms in a naphthalene ring is 10, and the number of ring atoms in a thiophene group is 5.

In the disclosure, the term “an alkyl group” refers to a linear alkyl group, a branched alkyl group, and/or a cyclic alkyl group. The number of carbon atoms in the alkyl group may range from 1 to 50, 1 to 30, 1 to 20, 1 to 10, or 1 to 6. The term containing the alkyl group, such as “a C₁₋₉ alkyl group”, refers to an alkyl group having 1 to 9 carbon atoms. The C₁₋₉ alkyl group at each occurrence is independently selected from a C₁ alkyl group, a C₂ alkyl group, a C₃ alkyl group, a C₄ alkyl group, a C₅ alkyl group, a C₆ alkyl group, a C₇ alkyl group, a C₈ alkyl group, or a C₉ alkyl group. Examples of the alkyl group include, but not limited to, methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butyhexyl, cyclohexyl, adamantane, and the like.

The term “an aryl group or an aromatic group” refers to an aromatic hydrocarbon group derived from a basis of an aromatic ring compound removing one hydrogen atom, which may be a single ring aryl group, a fused ring aryl group, or a polycyclic aryl group. For a polycyclic ring, at least one ring of the polycyclic ring is an aromatic ring system. For example, the term “a substituted or unsubstituted aryl group having 6 to 40 ring atoms” refers to an aryl group having 6 to 40 ring atoms, preferably a substituted or unsubstituted aryl group having 6 to 30 ring atoms, more preferably a substituted or unsubstituted aryl group having 6 to 18 ring atoms, most preferably a substituted or unsubstituted aryl group having 6 to 14 ring atoms, and the aryl group is optionally further substituted. Suitable examples include, but not limited to, benzene, biphenyl, triphenyl, naphthalene, anthracene, fluoranthene, phenanthrene, benzophenanthrene, perylene, naphthacene, pyrene, benzopyrene, acenaphthene, fluorine, and derivatives thereof. Understandably, aryl groups may further be disconnected by short non-aromatic units (for example, a non-hydrogenium atom contenting less than 10%, such as C, N, or O). In particular, acenaphthene, fluorene, or a system consisting of 9,9-diarylfluorene, triarylamine, and diaryl ether may further be included in the definition of the aryl group.

The term “an heteroaryl group or a heteroaromatic group” refers to a basis of an aryl group having at least one carbon atom substituted by a non-carbon atom, and the non-carbon atom may be N, O, S, or the like. For example, “a substituted or unsubstituted heteroaryl group having 5 to 40 ring atoms” refers to a heteroaryl group having 5 to 40 ring atoms, preferably a substituted or unsubstituted heteroaryl group having 6 to 30 ring atoms, more preferably a substituted or unsubstituted heteroaryl group having 6 to 18 ring atoms, most preferably a substituted or unsubstituted heteroaryl group having 6 to 14 ring atoms, and the heteroaryl group is optionally further substituted. Suitable examples of the heteroaryl group include, but not limited to, groups obtained from triazine, pyridine, pyrimidine, imidazole, furan, thiophene, benzofuran, benzothiophene, indole, carbazole, pyrrolimidazole, diketopyrrolopyrrole, thiophenopyrrole, thienothiophene, furanopyrrole, furanofuran, thienofuran, benzoisoxazole, benzisothiazole, benzimidazole, quinoline, isoquinoline, ortho-diazonaphthalene, quinoxaline, phenanthridine, perimidine, quinazoline, quinazolinone, dibenzothiophene, dibenzofuran, carbazole, and derivatives thereof.

In the disclosure, the term “*” connected to a single bond indicates a linking site or a fusing site.

In the disclosure, when a linking site in a group is not specified, it means that any of connectable sites in the group may be selected as the linking site.

In the disclosure, when a fusing site in a group is not specified, it means that any of fusible sites in the group may be selected as the fusing site. Preferably, two or more adjacent sites in the group form a fusing site.

In the disclosure, the term “adjacent groups” refers to the absence of substitutable sites between two substituent groups.

In the disclosure, the term “form a ring” refers to forming an aliphatic ring, an aromatic ring, a heteroaromatic ring, an aliphatic heterocycle, or any combinations of these rings.

A nitrogen-containing heterocyclic compound has a structure represented by formula (1):

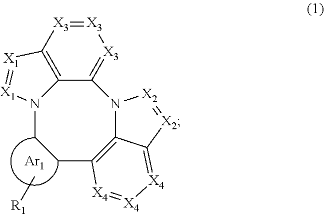

wherein,
Ar₁ is selected from a substituted or unsubstituted aromatic group having 6 to 40 ring atoms or a substituted or unsubstituted heteroaromatic group having 5 to 40 ring atoms;
X₁ at each occurrence is independently selected from CR₂ or N;
X₂ at each occurrence is independently selected from CR₃ or N;
X₃ at each occurrence is independently selected from CR₄ or N;
X₄ at each occurrence is independently selected from CR₅ or N;
R₁ to R₅ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups.

In one embodiment, R₁ to R₅ are independently selected from —H, -D, a linear alkyl group having 1 to 10 carbon atoms, a branched alkyl group having 3 to 10 carbon atoms, a cyclic alkyl group having 3 to 10 carbon atoms, an alkenyl group having 1 to 10 carbon atoms, a substituted or unsubstituted aromatic group having 6 to 18 ring atoms, or a substituted or unsubstituted heteroaromatic group having 6 to 18 ring atoms.

R₁ and X₄ are connected to form a ring or not form a ring together; two adjacent R₂ groups are connected to form a ring or not form a ring together; two adjacent R₃ groups are connected to form a ring or not form a ring together; two adjacent R₄ groups are connected to form a ring or not form a ring together; and two adjacent R₅ groups are connected to form a ring or not form a ring together.

In one embodiment, the term “form a ring” in the disclosure refers to forming an aromatic ring or a heteroaromatic ring with 5, 6, or 10 ring atoms. Further, the formed ring is a substituted or unsubstituted benzene or a substituted or unsubstituted naphthalene.

In one embodiment, two adjacent R₂ groups do not form a ring together, and two adjacent R₃ groups do not form a ring together. Further, the nitrogen-containing heterocyclic compound has a structure represented by any one of formulae (2-1) to (2-3):

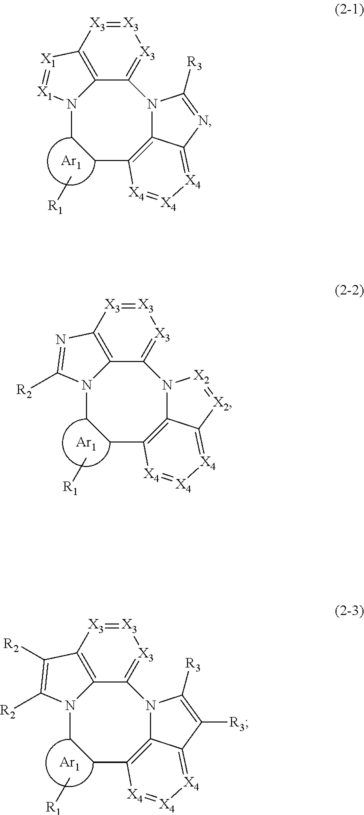

in which two adjacent X₁ groups in the formula (2-1) do not form a ring together, and two adjacent X₂ groups in the formula (2-2) do not form a ring together.

Further, the formula (2-1) and the formula (2-2) are independently selected from the following structures:

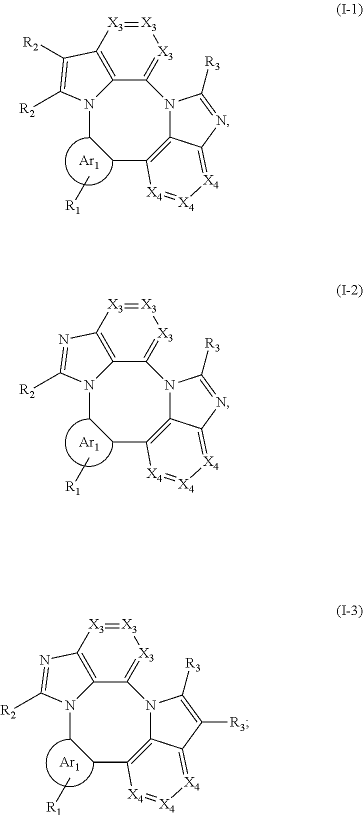

in which two adjacent R₂ groups in formula (I-1) do not form a ring together, and two adjacent R₃ groups in formula (I-3) do not form a ring together.

In one embodiment, two adjacent R₂ groups do not form a ring together, and two adjacent R₃ groups are connected to each other to form a six-membered ring; or, two adjacent R₂ groups are connected to form a six-membered ring, and two adjacent R₃ groups do not form a ring together. Further, the nitrogen-containing heterocyclic compound has a structure represented by any one of the following formulae (2-4) and (2-5):

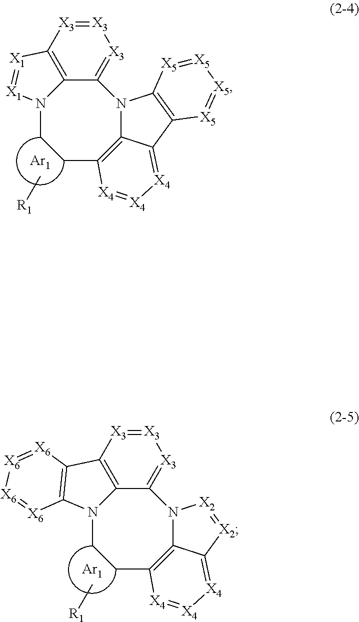

in which two adjacent X₁ groups in the formula (2-4) do not form a ring together; and two adjacent X₂ groups in the formula (2-5) do not form a ring together.

Further, the formulae (2-4) and (2-5) are independently selected from the following structures:

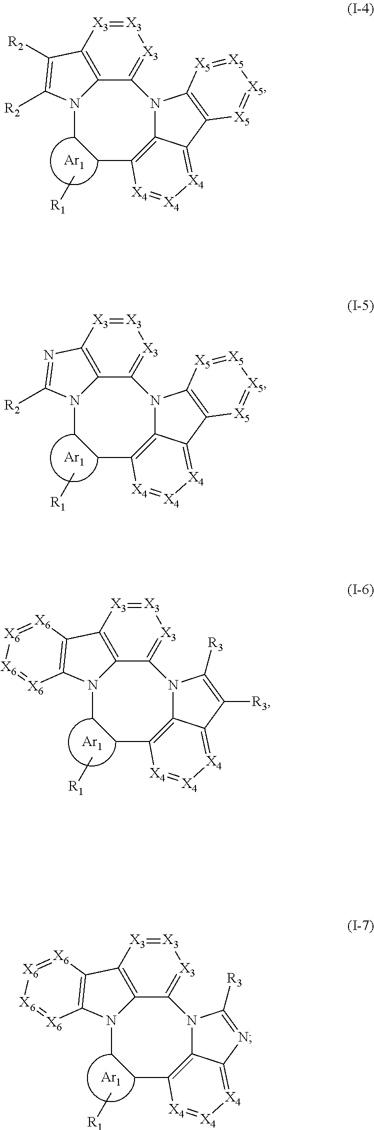

in which two adjacent R₂ groups in the formula (I-4) do not form a ring together, and two adjacent R₃ groups in the formula (I-6) do not form a ring together.

In one embodiment, two adjacent R₂ groups are connected to form a six-membered ring and two adjacent R₃ groups are connected to form a six-membered ring. Further, the nitrogen-containing heterocyclic compound has a structure represented by formula (2-6):

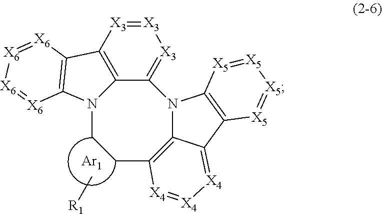

in which in the formulae (2-4), (2-5), (2-6), (I-4), (I-5), (I-6), and (I-7),
X₅ at each occurrence is independently selected from CR₆ or N;
X₆ at each occurrence is independently selected from CR₇ or N; and
R₆ and R₇ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; two adjacent R₆ groups are connected to form a ring or not form a ring together; and two adjacent R₇ groups are connected to form a ring or not form a ring together.

Preferably, the nitrogen-containing heterocyclic compound has a structure represented by the formula (2-6).

In one embodiment, at least one of two adjacent X₁ groups, two adjacent X₂ groups, two adjacent X₃ groups, and two adjacent X₄ groups in the formula (1) are independently connected to form a ring together. Further, the formed ring is an aromatic ring or a heteroaromatic ring with 6 or 10 ring atoms. Furthermore, the formed ring is a substituted or unsubstituted benzene or a substituted or unsubstituted naphthalene.

Preferably, the nitrogen-containing heterocyclic compound has a structure represented by any one of the following formulae (A-1) to (A-3):

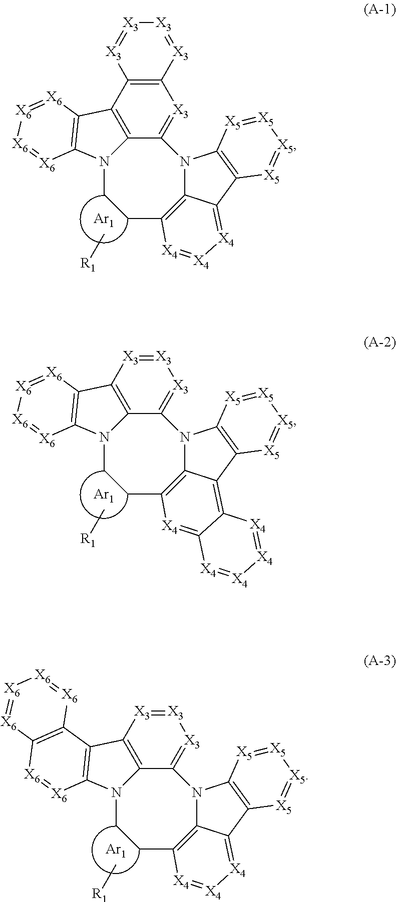

In one embodiment, Ar₁ is selected from a substituted or unsubstituted aromatic group having 6-18 ring atoms or a substituted or unsubstituted heteroaromatic group having 5-18 ring atoms.

In some preferable embodiments, Ar₁ is any one selected from a group consisting of following groups:

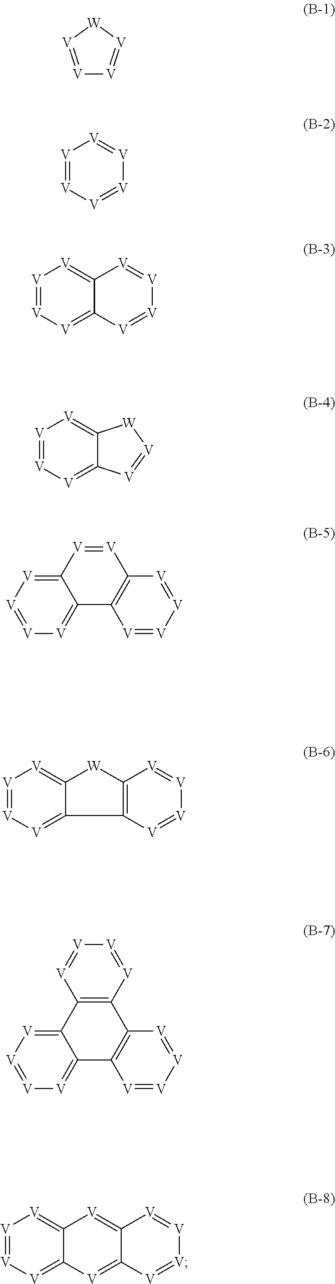

wherein,
V at each occurrence is independently selected from CR₁ or N, and at least one V is selected from CR₁;
W at each occurrence is independently selected from NR₈, PR₈, CR₈R₉, SiR₈R₉, O, S, S(═O)₂, or S(═O); and
R₈ and R₉ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups. It can be understood that when V is a linking site, V is C.

In one embodiment, R₈ and R₉ at each occurrence are independently selected from methyl or phenyl.

In one embodiment, W is independently selected from —NPh, —PPh, —C(CH₃)₂, —Si(CH₃)₂, O, S, S(═O)₂, or S(═O).

In one embodiment, Ar₁ is fused with other structures in the formula (1) through two adjacent C atoms.

Further, in one preferable embodiment, Ar₁ is any one selected from a group consisting of following groups:

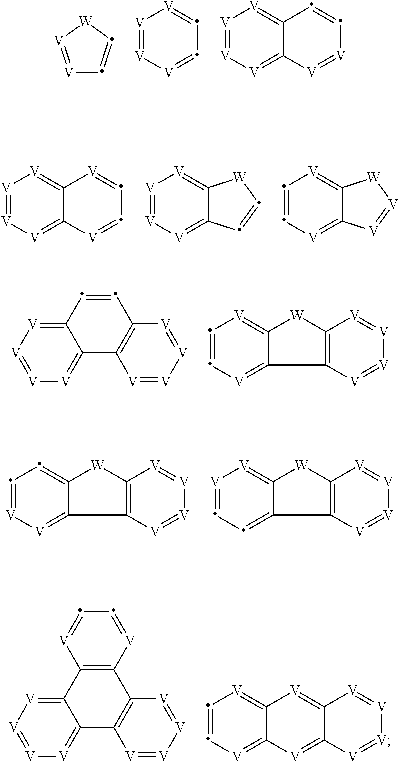

in which * indicates a linking site.

Preferably, the nitrogen-containing heterocyclic compound has a structure represented by any one of formulae (3-1) to (3-16):

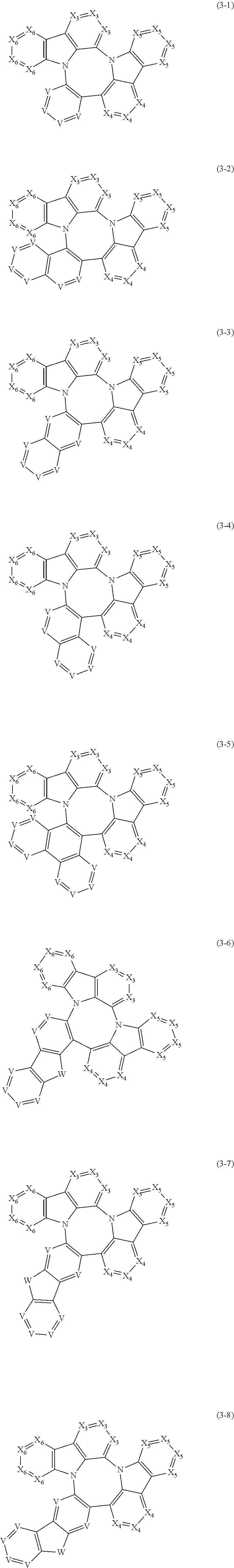

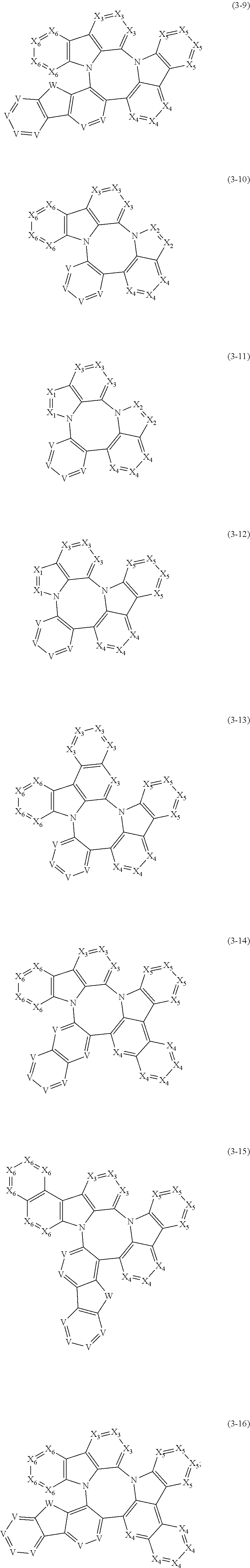

in which two adjacent X₂ groups in the formula (3-10) do not form a ring together; and two adjacent X₁ groups in the formulae (3-11) and (3-12) do not form a ring together.

In one embodiment, R₁ and X₄ are connected to form a ring together, for example, a five-membered ring or a six-membered ring.

Further, the nitrogen-containing heterocyclic compound has a structure represented by any one of formulae (4-1) and (4-2):

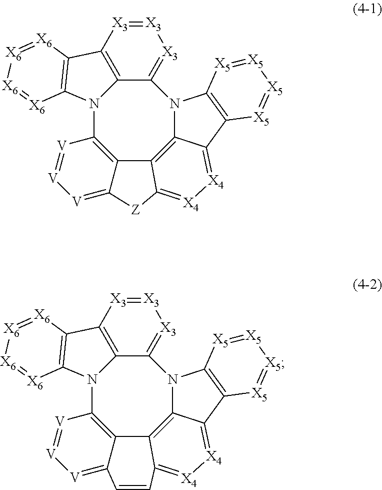

wherein,
Z at each occurrence is independently selected from NR₁₀, PR₁₀, CR₁₁R₁₂, SiR₁₁R₁₂, O, S, S(═O)₂, or S(═O);
R₁₀ to R₁₂ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
R₁₁ and R₁₂ are connected to form a ring or not form a ring together.

In one embodiment, R₁₁ and R₁₂ at each occurrence are independently selected from —H, -D, a linear alkyl group having 1 to 10 carbon atoms, a branched alkyl group having 3 to 10 carbon atoms, a cyclic alkyl group having 3 to 10 carbon atoms, a substituted or unsubstituted aromatic group having 5 to 20 ring atoms, or a substituted or unsubstituted heteroaromatic group having 5 to 20 ring atoms.

In one embodiment, R₁ to R₇ at each occurrence are independently from —H, -D, or a triazine group represented by formula (5).

In one embodiment, the nitrogen-containing heterocyclic compound has at least one triazine group represented by formula (5):
wherein,

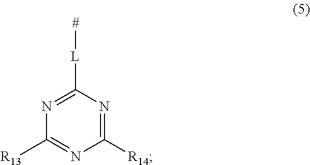

L is selected from a single bond, a substituted or unsubstituted aromatic group having 6 to 40 ring atoms, or a substituted or unsubstituted heteroaromatic group having 5 to 40 ring atoms;
R₁₃ and R₁₄ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
in which # indicates a linking site.

In one embodiment, at least one R₁ is selected from the formula (5). Further, only one R₁ is selected from the formula (5).

In one embodiment, at least one R₂ is selected from the formula (5). Further, only one R₂ is selected from the formula (5).

In one embodiment, at least one R₃ is selected from the formula (5). Further, only one R₃ is selected from the formula (5).

In one embodiment, at least one R₄ is selected from the formula (5). Further, only one R₄ is selected from the formula (5).

In one embodiment, at least one R₅ is selected from the formula (5). Further, only one R₅ is selected from the formula (5).

In one embodiment, at least one R₆ is selected from the formula (5). Further, only one R₆ is selected from formula (5).

In one embodiment, at least one R₇ is selected from the formula (5). Further, only one R₇ is selected from the formula (5).

In one embodiment, the nitrogen-containing heterocyclic compound has a structure represented by any one of the following structures:

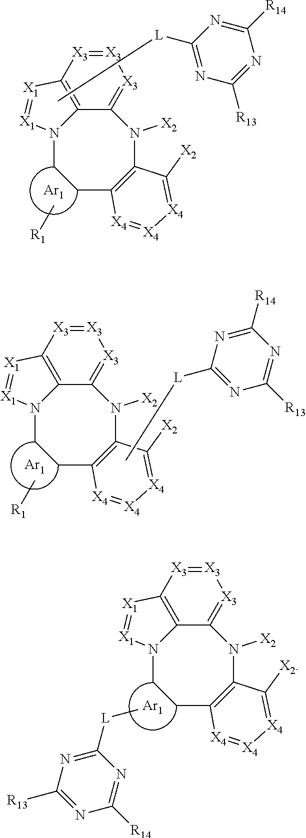

In one embodiment, L is selected from a single bond, phenyl, or naphthyl.

In one embodiment, R₁₃ and R₁₄ at each occurrence are independently selected from a group consisting of the following groups:

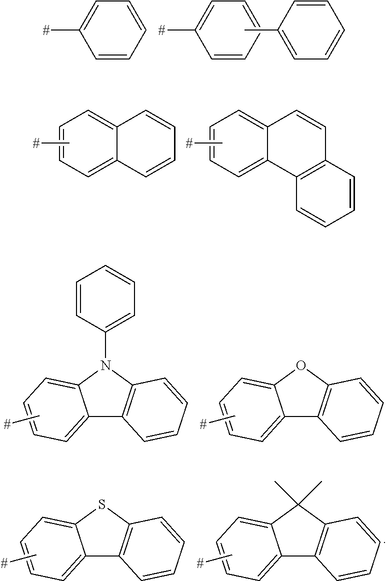

The nitrogen-containing heterocyclic compound provided in the disclosure has a structure represented by any one selected from a group consisting of the following structures, but not limited to these structures,

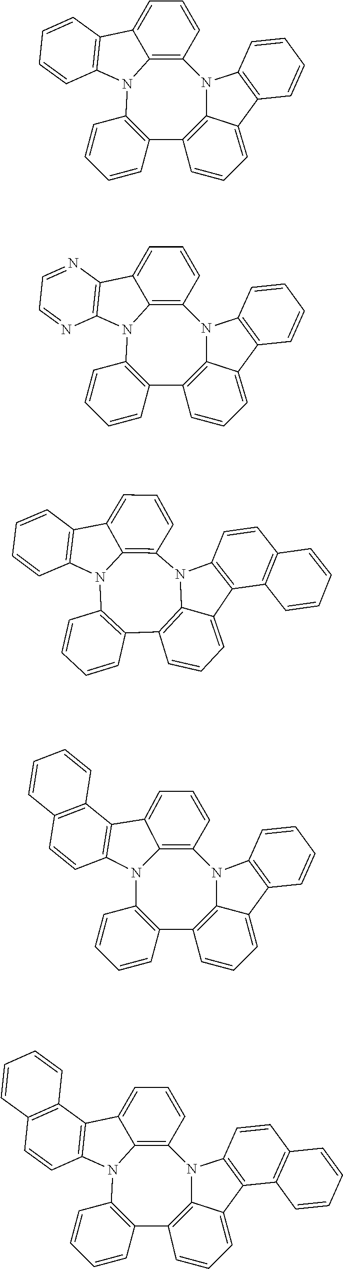 [SMILES: c1ccc2c(c1)c1cccc3c4ccccc4n4c5ccccc5c5cccc(c54)n2c13 ; c1ccc2c(c1)c1cccc3c4ccccc4n4c5nccnc5c5cccc(c54)n2c13 ; c1ccc2c(c1)ccc1c2c2cccc3c4ccccc4n4c5ccccc5c5cccc(c54)n1c32 ; c1ccc2c(c1)ccc1c2c2cccc3c2n1c1ccccc1c1cccc2c4ccccc4n3c21 ; c1ccc2c(c1)ccc1c2c2cccc3c4ccccc4n4c5ccc6ccccc6c5c5cccc(c54)n1c32]

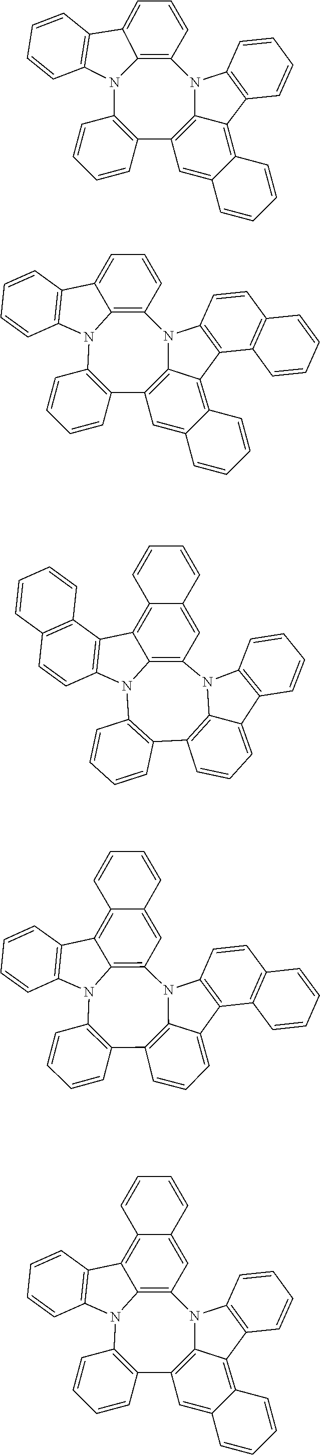 [SMILES: c1ccc2c(c1)cc1c3ccccc3n3c4ccccc4c4cccc(c43)n3c4ccccc4c2c13 ; c1ccc2c(c1)ccc1c2c2c3ccccc3cc3c4ccccc4n4c5ccccc5c5cccc(c54)n1c32 ; c1ccc2c(c1)ccc1c2c2c3ccccc3cc3c2n1c1ccccc1c1cccc2c4ccccc4n3c21 ; c1ccc2c(c1)ccc1c2c2cccc3c4ccccc4n4c5ccccc5c5c6ccccc6cc(c54)n1c32 ; c1ccc2c(c1)cc1c3ccccc3n3c4ccccc4c4c5ccccc5cc(c43)n3c4ccccc4c2c13]

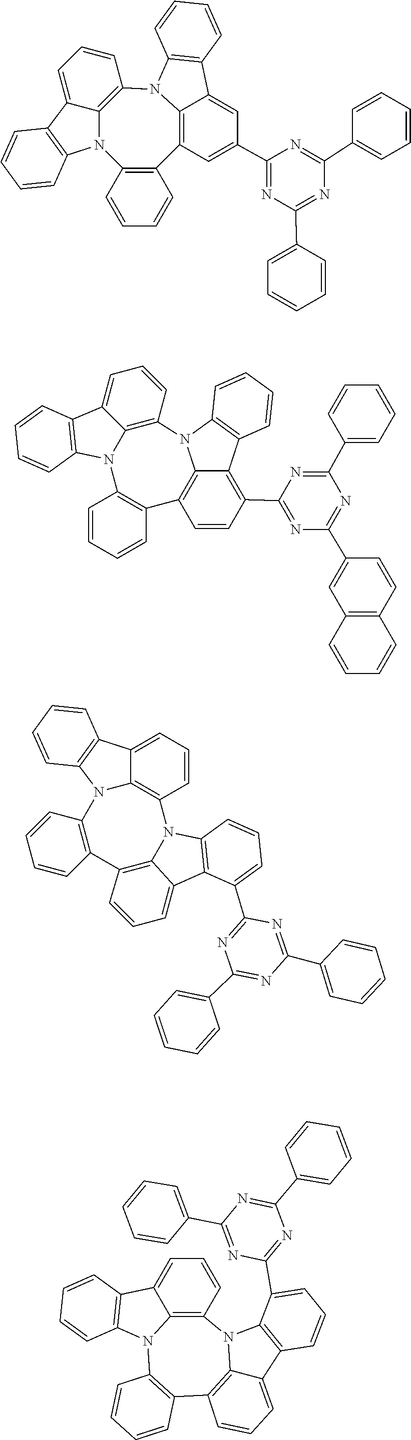 [SMILES: c1ccc(-c2nc(-c3ccccc3)nc(-c3cc4c5ccccc5n5c6cccc7c8ccccc8n(c8ccccc8c(c3)c45)c76)n2)cc1 ; c1ccc(-c2nc(-c3ccc4ccccc4c3)nc(-c3ccc4c5ccccc5n5c6ccccc6c6cccc(c65)n5c6ccccc6c3c45)n2)cc1 ; c1ccc(-c2nc(-c3ccccc3)nc(-c3cccc4c3c3cccc5c6ccccc6n6c7ccccc7c7cccc(c76)n4c53)n2)cc1 ; c1ccc(-c2nc(-c3ccccc3)nc(-c3cccc4c5cccc6c7ccccc7n7c8ccccc8c8cccc(c87)n(c34)c65)n2)cc1]

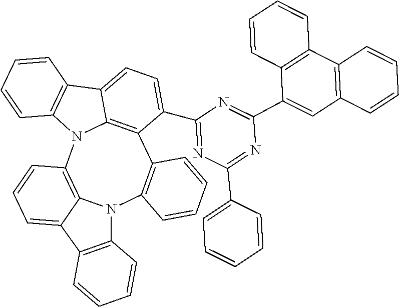 [SMILES: c1ccc(-c2nc(-c3cc4ccccc4c4ccccc34)nc(-c3ccc4c5ccccc5n5c6cccc7c8ccccc8n(c8ccccc8c3c45)c76)n2)cc1]

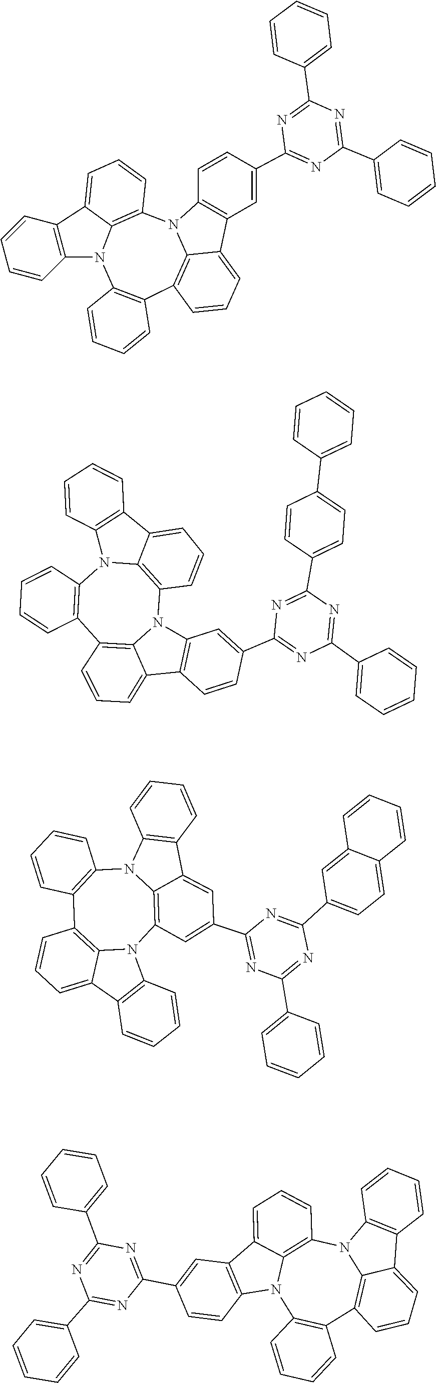 [SMILES: c1ccc(-c2nc(-c3ccccc3)nc(-c3ccc4c(c3)c3cccc5c6ccccc6n6c7ccccc7c7cccc(c76)n4c35)n2)cc1 ; c1ccc(-c2ccc(-c3nc(-c4ccccc4)nc(-c4ccc5c6cccc7c8ccccc8n8c9ccccc9c9cccc(c98)n(c5c4)c67)n3)cc2)cc1 ; c1ccc(-c2nc(-c3ccc4ccccc4c3)nc(-c3cc4c5ccccc5n5c6ccccc6c6cccc7c8ccccc8n(c(c3)c45)c76)n2)cc1 ; c1ccc(-c2nc(-c3ccccc3)nc(-c3ccc4c(c3)c3cccc5c3n4c3ccccc3c3cccc4c6ccccc6n5c43)n2)cc1]

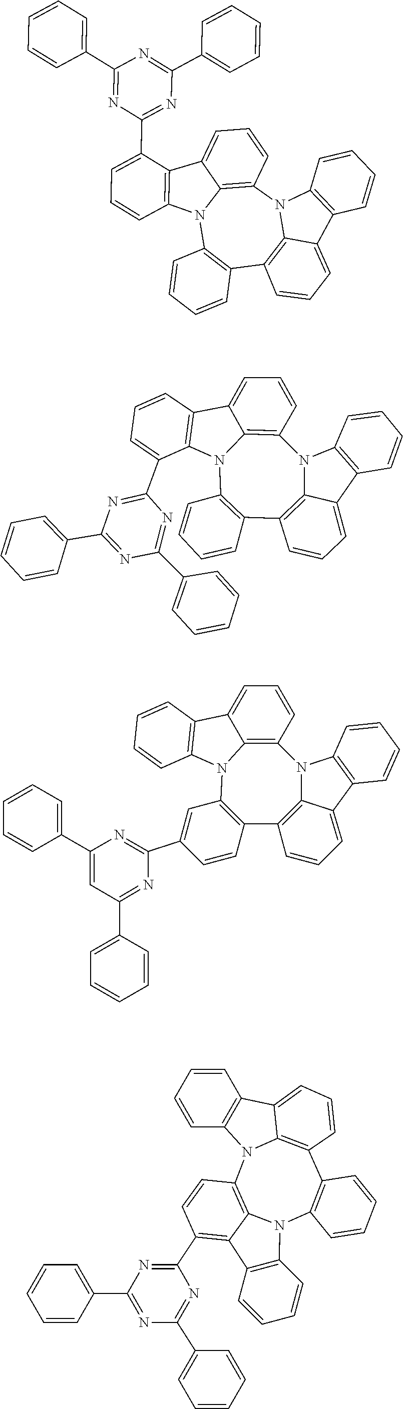 [SMILES: c1ccc(-c2nc(-c3ccccc3)nc(-c3cccc4c3c3cccc5c3n4c3ccccc3c3cccc4c6ccccc6n5c43)n2)cc1 ; c1ccc(-c2nc(-c3ccccc3)nc(-c3cccc4c5cccc6c5n(c5ccccc5c5cccc7c8ccccc8n6c75)c34)n2)cc1 ; c1ccc(-c2cc(-c3ccccc3)nc(-c3ccc4c5cccc6c7ccccc7n(c7cccc8c9ccccc9n(c4c3)c87)c65)n2)cc1 ; c1ccc(-c2nc(-c3ccccc3)nc(-c3ccc4c5c3c3ccccc3n5c3ccccc3c3cccc5c6ccccc6n4c53)n2)cc1]

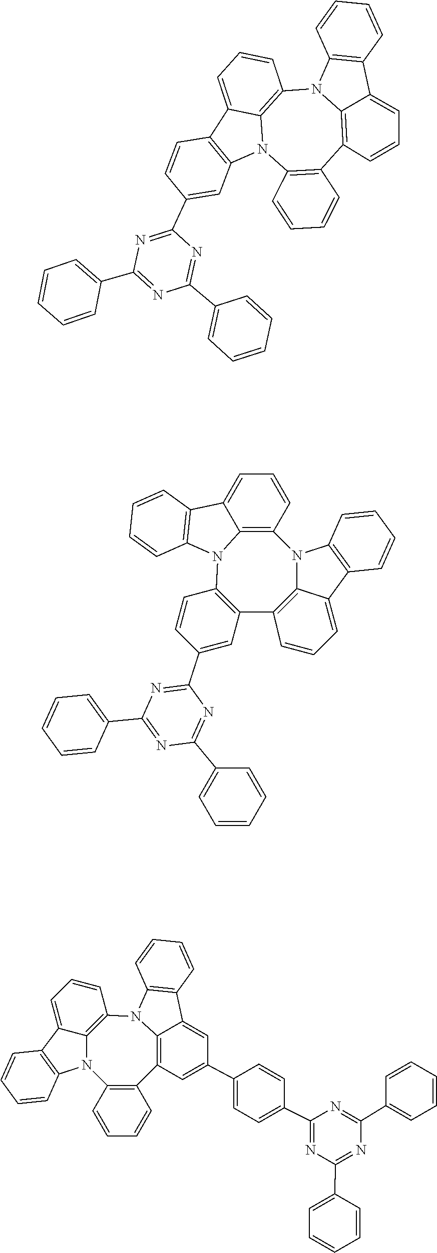 [SMILES: c1ccc(-c2nc(-c3ccccc3)nc(-c3ccc4c5cccc6c5n(c5ccccc5c5cccc7c8ccccc8n6c75)c4c3)n2)cc1 ; c1ccc(-c2nc(-c3ccccc3)nc(-c3ccc4c(c3)c3cccc5c6ccccc6n(c6cccc7c8ccccc8n4c76)c53)n2)cc1 ; c1ccc(-c2nc(-c3ccccc3)nc(-c3ccc(-c4cc5c6ccccc6n6c7cccc8c9ccccc9n(c9ccccc9c(c4)c56)c87)cc3)n2)cc1]

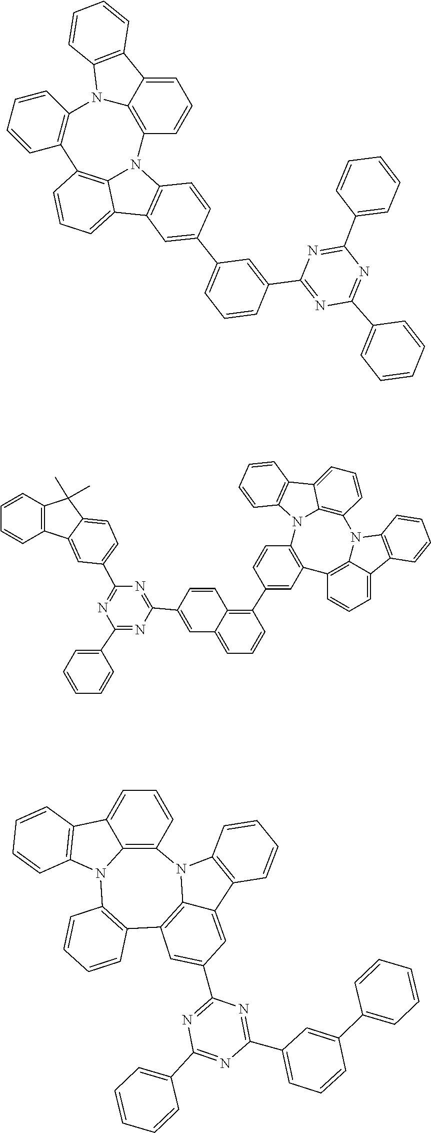 [SMILES: c1ccc(-c2nc(-c3ccccc3)nc(-c3cccc(-c4ccc5c(c4)c4cccc6c7ccccc7n7c8ccccc8c8cccc(c87)n5c46)c3)n2)cc1 ; CC1(C)c2ccccc2-c2cc(-c3nc(-c4ccccc4)nc(-c4ccc5c(-c6ccc7c(c6)c6cccc8c9ccccc9n(c9cccc%10c%11ccccc%11n7c%109)c86)cccc5c4)n3)ccc21 ; c1ccc(-c2cccc(-c3nc(-c4ccccc4)nc(-c4cc5c6ccccc6n6c7cccc8c9ccccc9n(c9ccccc9c(c4)c56)c87)n3)c2)cc1]

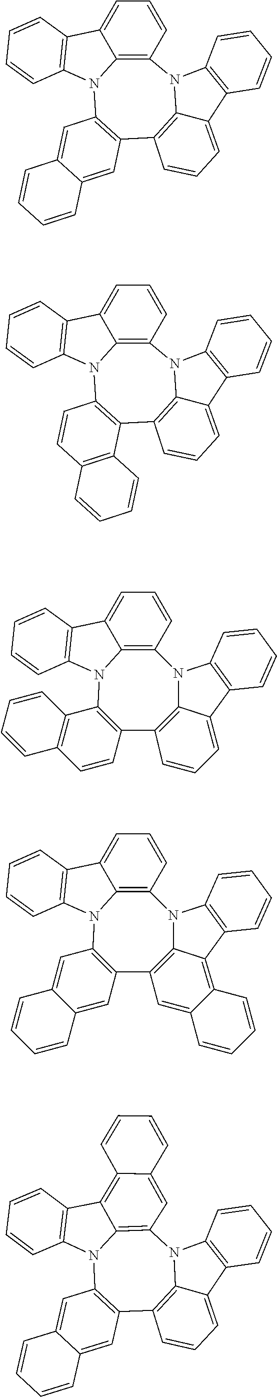 [SMILES: c1ccc2cc3c(cc2c1)c1cccc2c4ccccc4n(c4cccc5c6ccccc6n3c54)c21 ; c1ccc2c(c1)ccc1c2c2cccc3c4ccccc4n(c4cccc5c6ccccc6n1c54)c32 ; c1ccc2c(c1)ccc1c3cccc4c5ccccc5n(c5cccc6c7ccccc7n(c21)c65)c43 ; c1ccc2cc3c(cc2c1)c1cc2ccccc2c2c4ccccc4n(c4cccc5c6ccccc6n3c54)c12 ; c1ccc2cc3c(cc2c1)c1cccc2c4ccccc4n(c4cc5ccccc5c5c6ccccc6n3c54)c21]

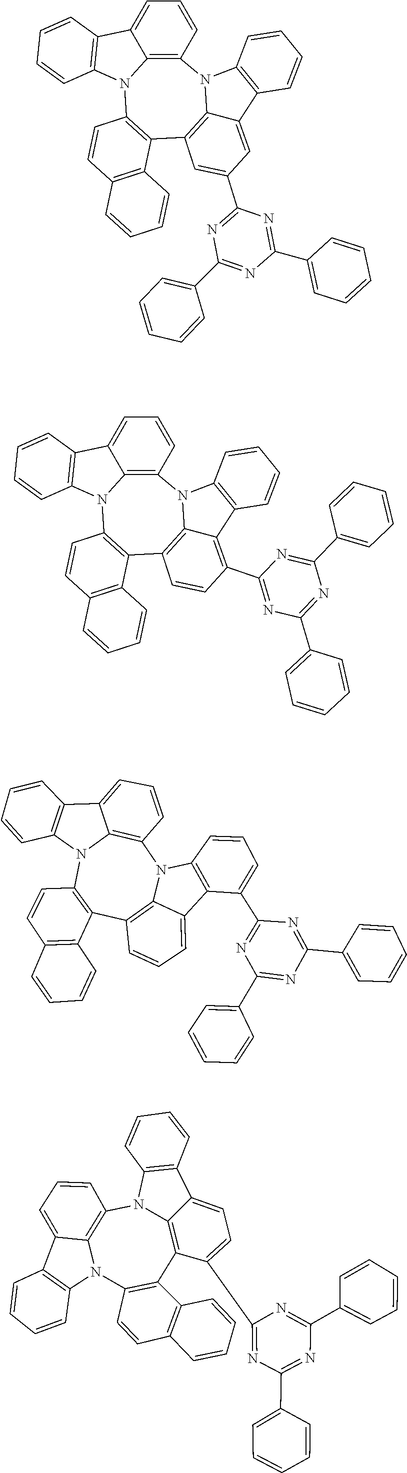 [SMILES: c1ccc(-c2nc(-c3ccccc3)nc(-c3cc4c5ccccc5n5c6cccc7c8ccccc8n(c8ccc9ccccc9c8c(c3)c45)c76)n2)cc1 ; c1ccc(-c2nc(-c3ccccc3)nc(-c3ccc4c5c6ccccc6ccc5n5c6ccccc6c6cccc(c65)n5c6ccccc6c3c45)n2)cc1 ; c1ccc(-c2nc(-c3ccccc3)nc(-c3cccc4c3c3cccc5c6c7ccccc7ccc6n6c7ccccc7c7cccc(c76)n4c35)n2)cc1 ; c1ccc(-c2nc(-c3ccccc3)nc(-c3ccc4c5ccccc5n5c6cccc7c8ccccc8n(c8ccc9ccccc9c8c3c45)c76)n2)cc1]

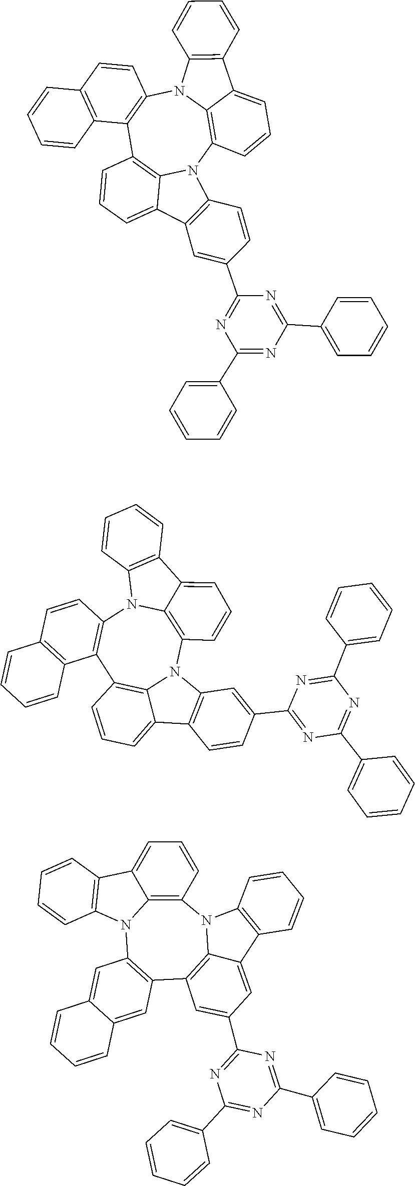 [SMILES: c1ccc(-c2nc(-c3ccccc3)nc(-c3ccc4c(c3)c3cccc5c6c7ccccc7ccc6n6c7ccccc7c7cccc(c76)n4c35)n2)cc1 ; c1ccc(-c2nc(-c3ccccc3)nc(-c3ccc4c5cccc6c7c8ccccc8ccc7n7c8ccccc8c8cccc(c87)n(c4c3)c56)n2)cc1 ; c1ccc(-c2nc(-c3ccccc3)nc(-c3cc4c5ccccc5n5c6cccc7c8ccccc8n(c8cc9ccccc9cc8c(c3)c45)c76)n2)cc1]

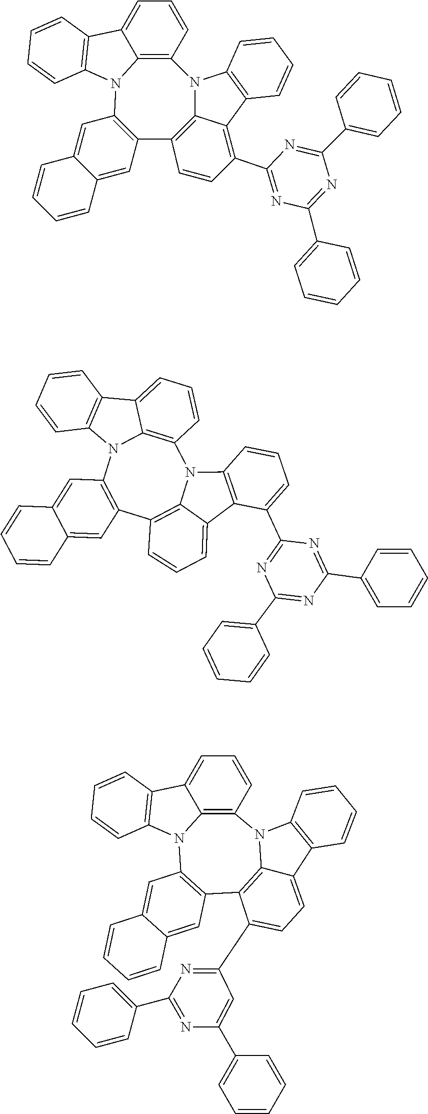 [SMILES: c1ccc(-c2nc(-c3ccccc3)nc(-c3ccc4c5cc6ccccc6cc5n5c6ccccc6c6cccc(c65)n5c6ccccc6c3c45)n2)cc1 ; c1ccc(-c2nc(-c3ccccc3)nc(-c3cccc4c3c3cccc5c6cc7ccccc7cc6n6c7ccccc7c7cccc(c76)n4c53)n2)cc1 ; c1ccc(-c2cc(-c3ccc4c5ccccc5n5c6cccc7c8ccccc8n(c8cc9ccccc9cc8c3c45)c76)nc(-c3ccccc3)n2)cc1]

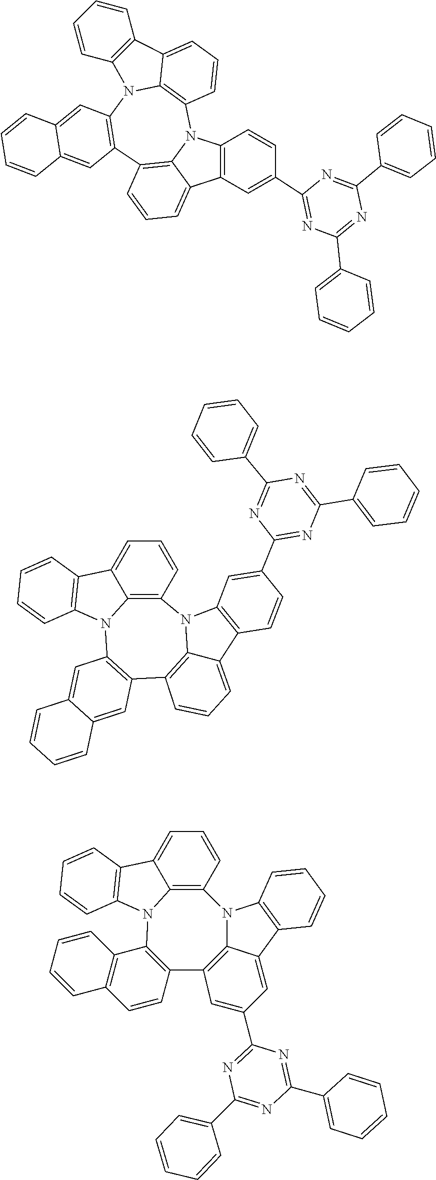 [SMILES: c1ccc(-c2nc(-c3ccccc3)nc(-c3ccc4c(c3)c3cccc5c6cc7ccccc7cc6n6c7ccccc7c7cccc(c76)n4c35)n2)cc1 ; c1ccc(-c2nc(-c3ccccc3)nc(-c3ccc4c5cccc6c7cc8ccccc8cc7n7c8ccccc8c8cccc(c87)n(c4c3)c56)n2)cc1 ; c1ccc(-c2nc(-c3ccccc3)nc(-c3cc4c5ccccc5n5c6cccc7c8ccccc8n(c8c9ccccc9ccc8c(c3)c45)c76)n2)cc1]

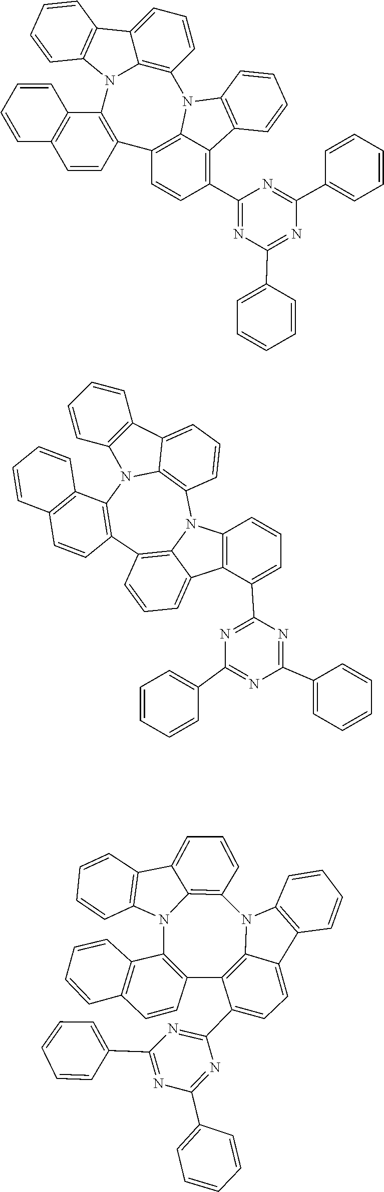 [SMILES: c1ccc(-c2nc(-c3ccccc3)nc(-c3ccc4c5ccc6ccccc6c5n5c6ccccc6c6cccc(c65)n5c6ccccc6c3c45)n2)cc1 ; c1ccc(-c2nc(-c3ccccc3)nc(-c3cccc4c3c3cccc5c6ccc7ccccc7c6n6c7ccccc7c7cccc(c76)n4c53)n2)cc1 ; c1ccc(-c2nc(-c3ccccc3)nc(-c3ccc4c5ccccc5n5c6cccc7c8ccccc8n(c8c9ccccc9ccc8c3c45)c76)n2)cc1]

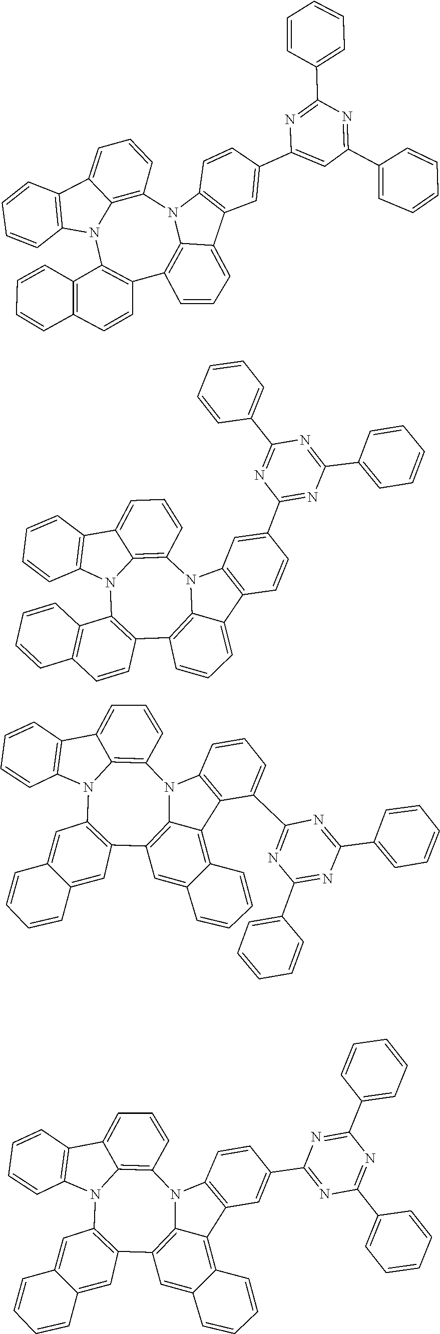

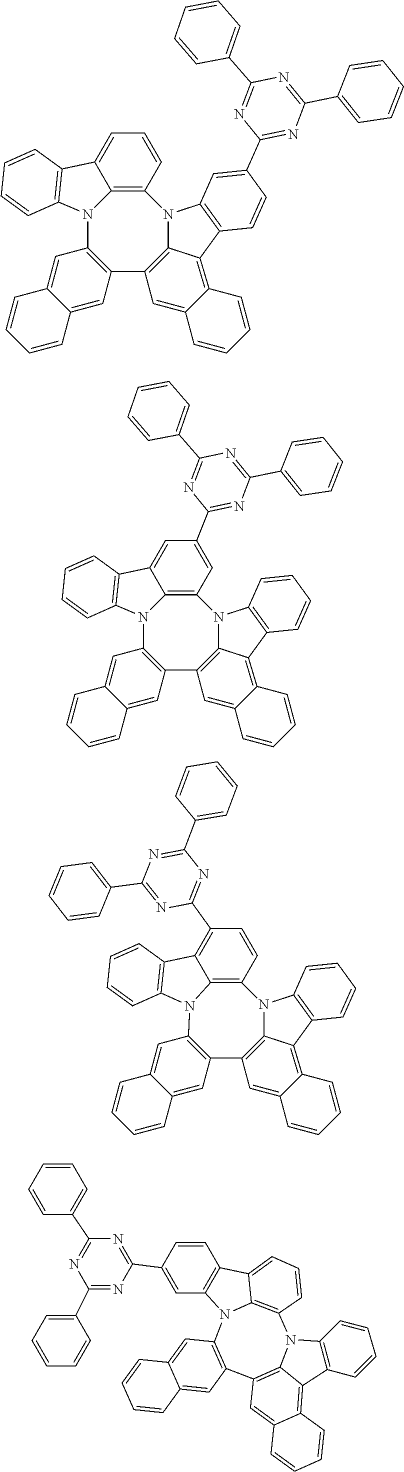

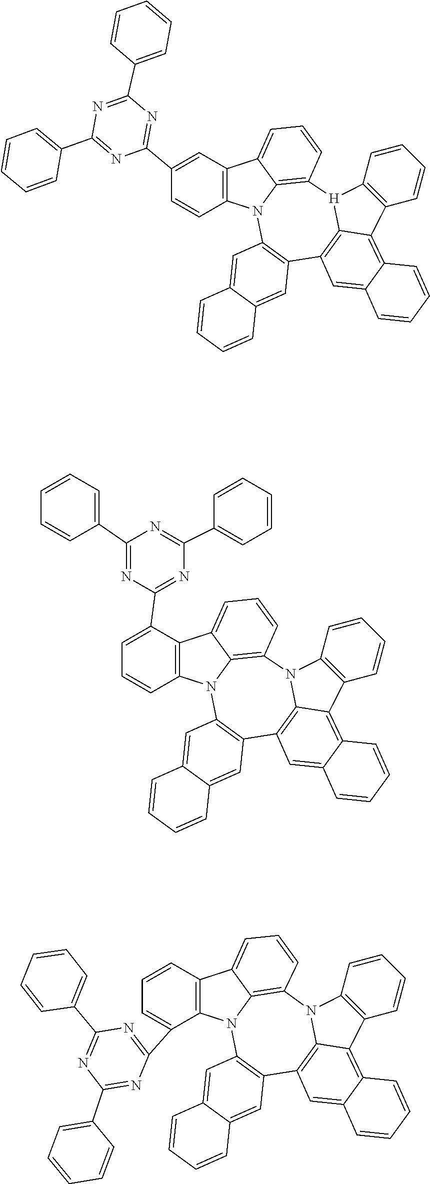

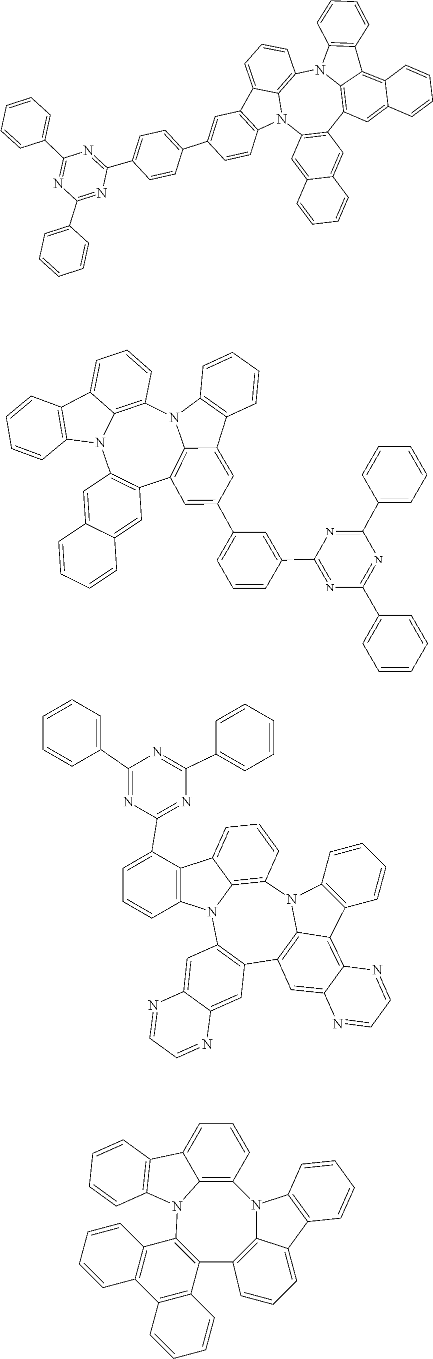 [SMILES: c1ccc(-c2nc(-c3ccccc3)nc(-c3ccc(-c4ccc5c(c4)c4cccc6c4n5c4cc5ccccc5cc4c4cc5ccccc5c5c7ccccc7n6c45)cc3)n2)cc1 ; c1ccc(-c2nc(-c3ccccc3)nc(-c3cccc(-c4cc5c6ccccc6n6c7cccc8c9ccccc9n(c9cc%10ccccc%10cc9c(c4)c56)c87)c3)n2)cc1 ; c1ccc(-c2nc(-c3ccccc3)nc(-c3cccc4c3c3cccc5c3n4c3cc4nccnc4cc3c3cc4nccnc4c4c6ccccc6n5c34)n2)cc1 ; c1ccc2c(c1)c1ccccc1c1c2c2cccc3c4ccccc4n(c4cccc5c6ccccc6n1c54)c32]

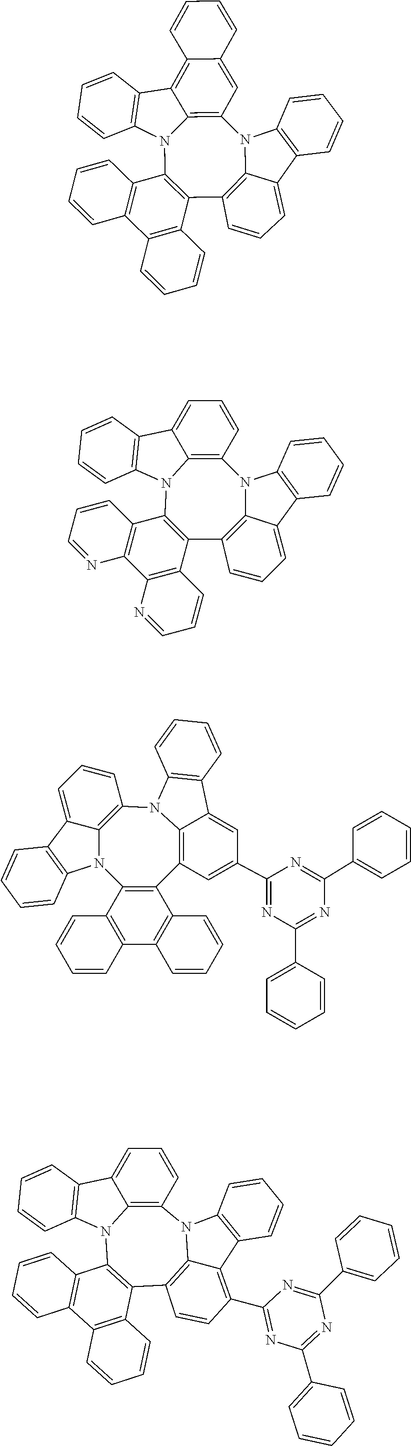 [SMILES: c1ccc2c(c1)cc1c3c2c2ccccc2n3c2c3ccccc3c3ccccc3c2c2cccc3c4ccccc4n1c32 ; c1cnc2c(c1)c1c3cccc4c5ccccc5n(c5cccc6c7ccccc7n(c1c1cccnc12)c65)c43 ; c1ccc(-c2nc(-c3ccccc3)nc(-c3cc4c5ccccc5n5c6cccc7c8ccccc8n(c8c9ccccc9c9ccccc9c8c(c3)c45)c76)n2)cc1 ; c1ccc(-c2nc(-c3ccccc3)nc(-c3ccc4c5c6ccccc6c6ccccc6c5n5c6ccccc6c6cccc(c65)n5c6ccccc6c3c45)n2)cc1]

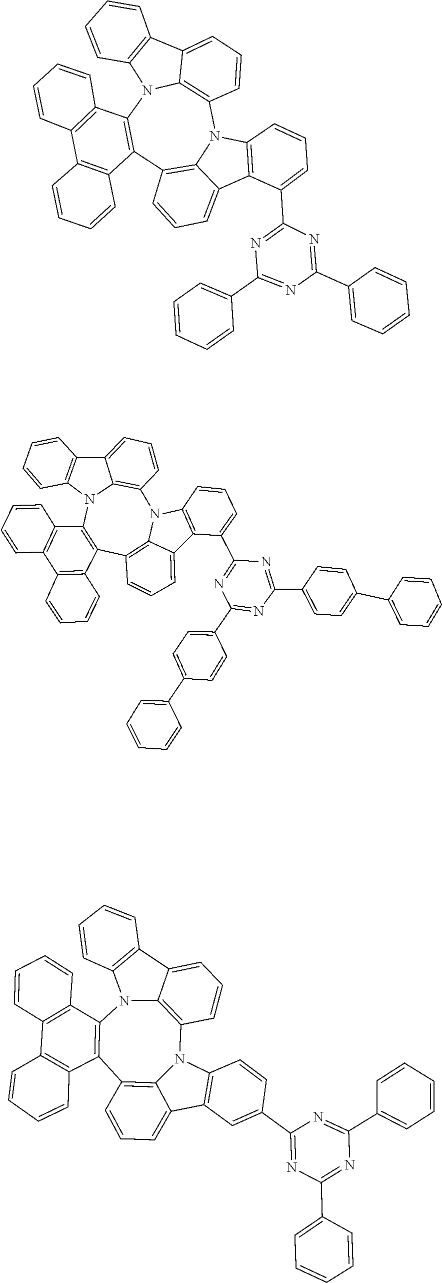 [SMILES: c1ccc(-c2nc(-c3ccccc3)nc(-c3cccc4c3c3cccc5c6c7ccccc7c7ccccc7c6n6c7ccccc7c7cccc(c76)n4c35)n2)cc1 ; c1ccc(-c2ccc(-c3nc(-c4ccc(-c5ccccc5)cc4)nc(-c4cccc5c4c4cccc6c7c8ccccc8c8ccccc8c7n7c8ccccc8c8cccc(c87)n5c46)n3)cc2)cc1 ; c1ccc(-c2nc(-c3ccccc3)nc(-c3ccc4c(c3)c3cccc5c6c7ccccc7c7ccccc7c6n6c7ccccc7c7cccc(c76)n4c35)n2)cc1]

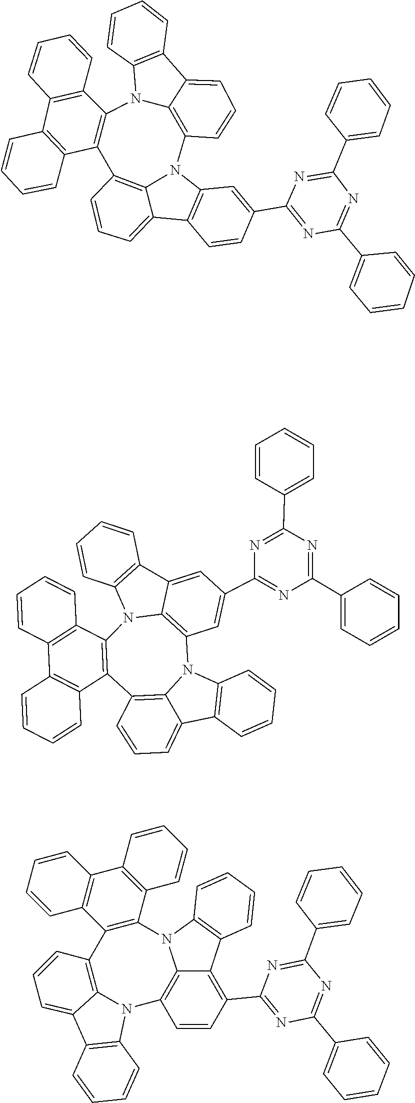 [SMILES: c1ccc(-c2nc(-c3ccccc3)nc(-c3ccc4c5cccc6c7c8ccccc8c8ccccc8c7n7c8ccccc8c8cccc(c87)n(c4c3)c56)n2)cc1 ; c1ccc(-c2nc(-c3ccccc3)nc(-c3cc4c5ccccc5n5c6c7ccccc7c7ccccc7c6c6cccc7c8ccccc8n(c(c3)c45)c76)n2)cc1 ; c1ccc(-c2nc(-c3ccccc3)nc(-c3ccc4c5c3c3ccccc3n5c3c5ccccc5c5ccccc5c3c3cccc5c6ccccc6n4c53)n2)cc1]

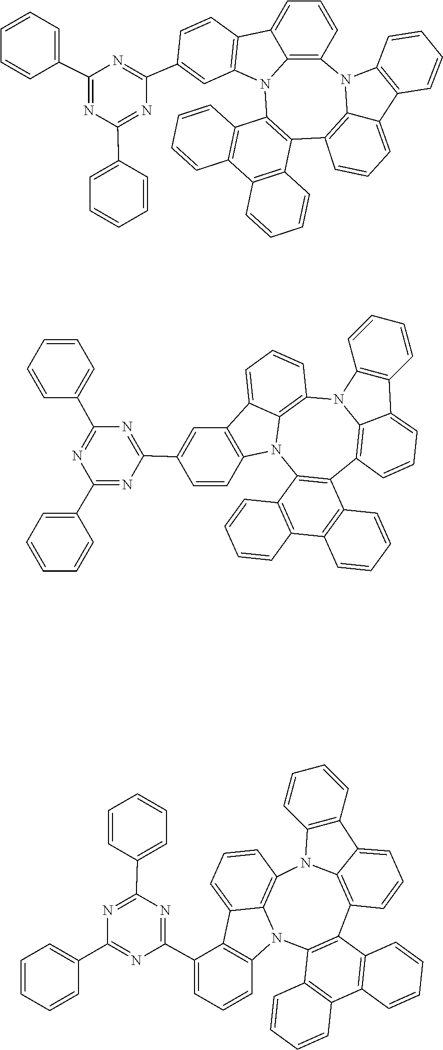 [SMILES: c1ccc(-c2nc(-c3ccccc3)nc(-c3ccc4c5cccc6c5n(c4c3)c3c4ccccc4c4ccccc4c3c3cccc4c5ccccc5n6c43)n2)cc1 ; c1ccc(-c2nc(-c3ccccc3)nc(-c3ccc4c(c3)c3cccc5c3n4c3c4ccccc4c4ccccc4c3c3cccc4c6ccccc6n5c43)n2)cc1 ; c1ccc(-c2nc(-c3ccccc3)nc(-c3cccc4c3c3cccc5c3n4c3c4ccccc4c4ccccc4c3c3cccc4c6ccccc6n5c43)n2)cc1]

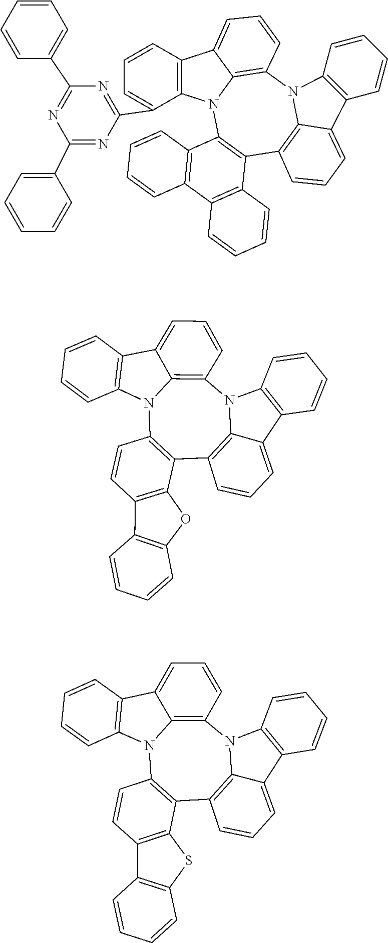 [SMILES: c1ccc(-c2nc(-c3ccccc3)nc(-c3cccc4c5cccc6c5n(c34)c3c4ccccc4c4ccccc4c3c3cccc4c5ccccc5n6c43)n2)cc1 ; c1ccc2c(c1)oc1c2ccc2c1c1cccc3c4ccccc4n(c4cccc5c6ccccc6n2c54)c31 ; c1ccc2c(c1)sc1c2ccc2c1c1cccc3c4ccccc4n(c4cccc5c6ccccc6n2c54)c31]

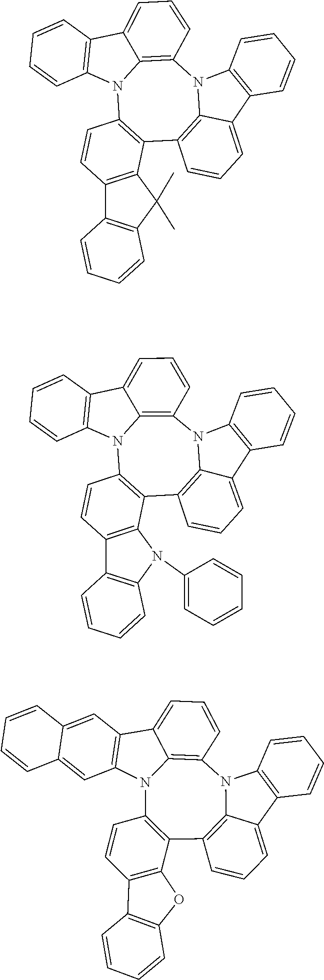 [SMILES: CC1(C)c2ccccc2-c2ccc3c(c21)c1cccc2c4ccccc4n(c4cccc5c6ccccc6n3c54)c21 ; c1ccc(-n2c3ccccc3c3ccc4c(c5cccc6c7ccccc7n(c7cccc8c9ccccc9n4c87)c65)c32)cc1 ; c1ccc2cc3c(cc2c1)c1cccc2c1n3c1ccc3c4ccccc4oc3c1c1cccc3c4ccccc4n2c31]

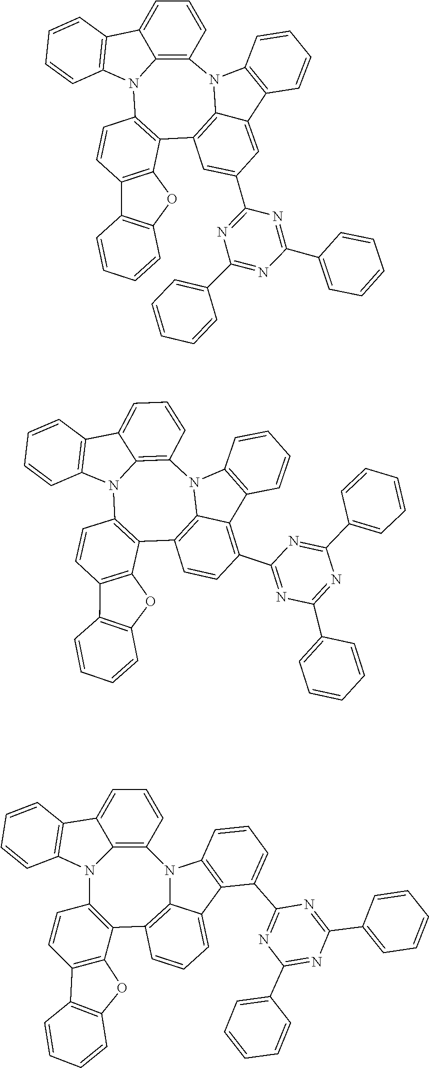 [SMILES: c1ccc(-c2nc(-c3ccccc3)nc(-c3cc4c5ccccc5n5c6cccc7c8ccccc8n(c8ccc9c%10ccccc%10oc9c8c(c3)c45)c76)n2)cc1 ; c1ccc(-c2nc(-c3ccccc3)nc(-c3ccc4c5c6oc7ccccc7c6ccc5n5c6ccccc6c6cccc(c65)n5c6ccccc6c3c45)n2)cc1 ; c1ccc(-c2nc(-c3ccccc3)nc(-c3cccc4c3c3cccc5c6c7oc8ccccc8c7ccc6n6c7ccccc7c7cccc(c76)n4c35)n2)cc1]

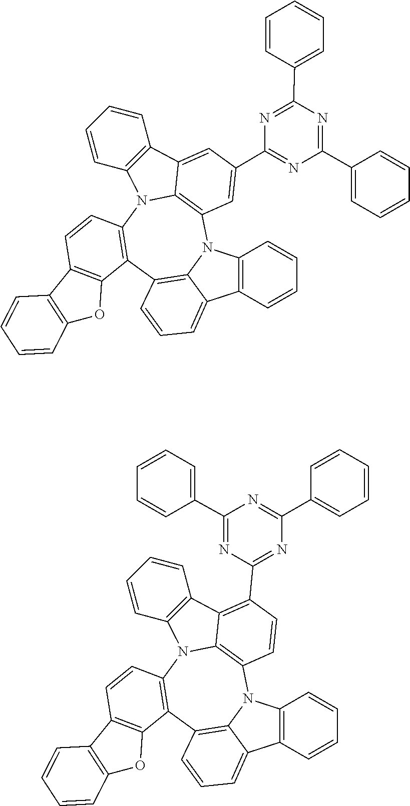 [SMILES: c1ccc(-c2nc(-c3ccccc3)nc(-c3cc4c5ccccc5n5c6ccc7c8ccccc8oc7c6c6cccc7c8ccccc8n(c(c3)c45)c76)n2)cc1 ; c1ccc(-c2nc(-c3ccccc3)nc(-c3ccc4c5c3c3ccccc3n5c3ccc5c6ccccc6oc5c3c3cccc5c6ccccc6n4c53)n2)cc1]

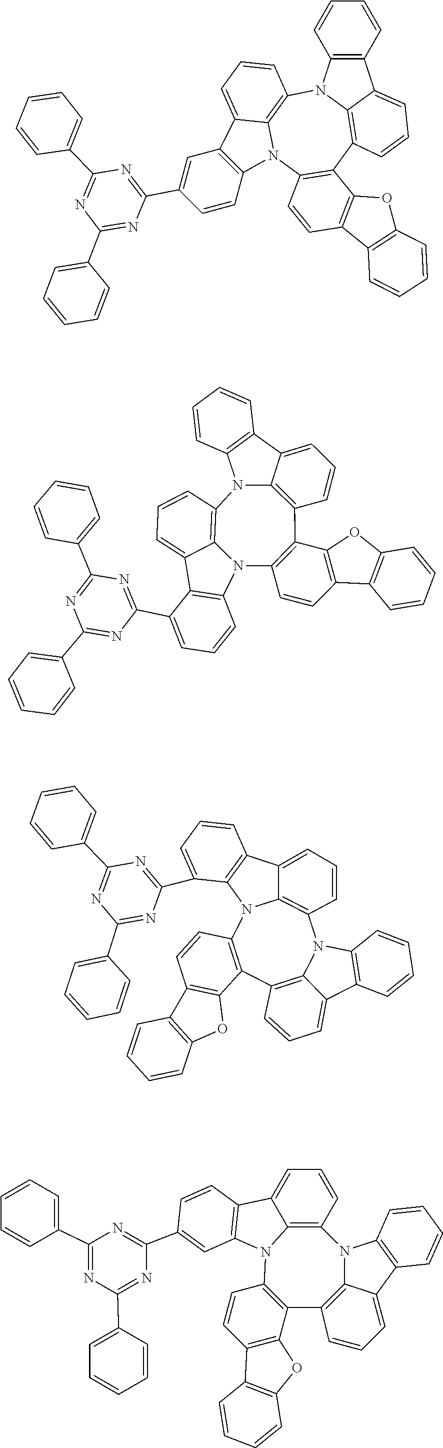 [SMILES: c1ccc(-c2nc(-c3ccccc3)nc(-c3ccc4c(c3)c3cccc5c3n4c3ccc4c6ccccc6oc4c3c3cccc4c6ccccc6n5c43)n2)cc1 ; c1ccc(-c2nc(-c3ccccc3)nc(-c3cccc4c3c3cccc5c3n4c3ccc4c6ccccc6oc4c3c3cccc4c6ccccc6n5c43)n2)cc1 ; c1ccc(-c2nc(-c3ccccc3)nc(-c3cccc4c5cccc6c5n(c5ccc7c8ccccc8oc7c5c5cccc7c8ccccc8n6c75)c34)n2)cc1 ; c1ccc(-c2nc(-c3ccccc3)nc(-c3ccc4c5cccc6c5n(c4c3)c3ccc4c5ccccc5oc4c3c3cccc4c5ccccc5n6c43)n2)cc1]

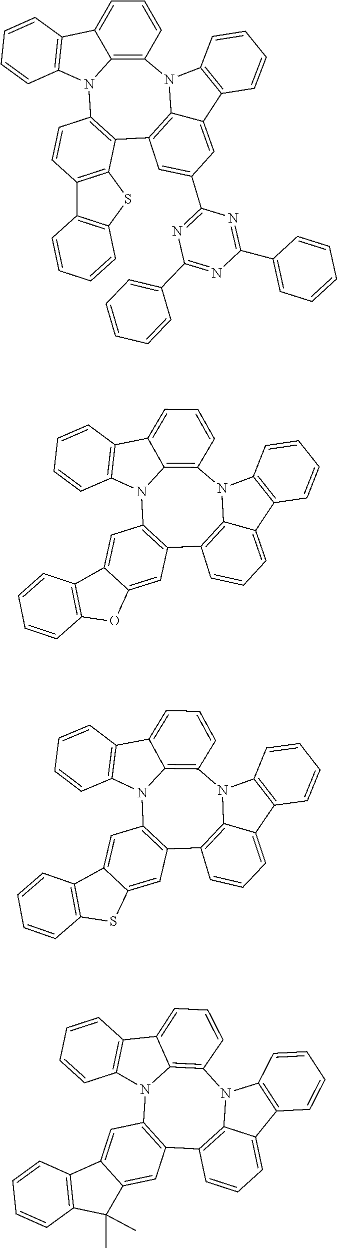 [SMILES: c1ccc(-c2nc(-c3ccccc3)nc(-c3cc4c5ccccc5n5c6cccc7c8ccccc8n(c8ccc9c%10ccccc%10sc9c8c(c3)c45)c76)n2)cc1 ; c1ccc2c(c1)oc1cc3c4cccc5c6ccccc6n(c6cccc7c8ccccc8n(c3cc12)c76)c54 ; c1ccc2c(c1)sc1cc3c4cccc5c6ccccc6n(c6cccc7c8ccccc8n(c3cc12)c76)c54 ; CC1(C)c2ccccc2-c2cc3c(cc21)c1cccc2c4ccccc4n(c4cccc5c6ccccc6n3c54)c21]

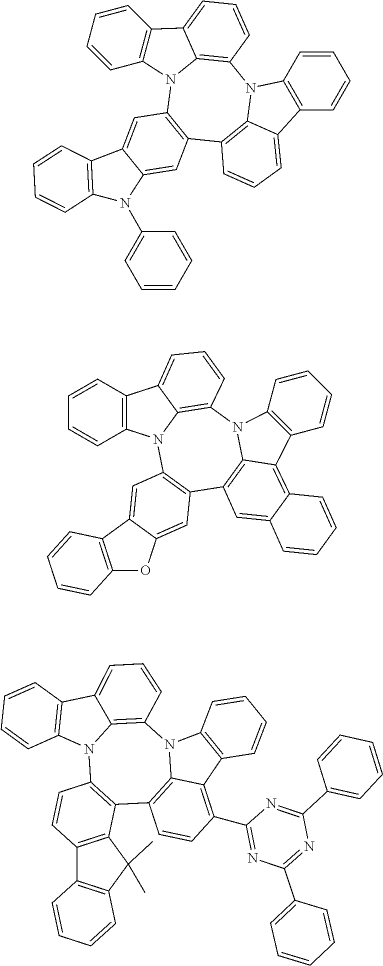 [SMILES: c1ccc(-n2c3ccccc3c3cc4c(cc32)c2cccc3c5ccccc5n(c5cccc6c7ccccc7n4c65)c32)cc1 ; c1ccc2c(c1)cc1c3cc4oc5ccccc5c4cc3n3c4ccccc4c4cccc(c43)n3c4ccccc4c2c13 ; CC1(C)c2ccccc2-c2ccc3c(c21)c1ccc(-c2nc(-c4ccccc4)nc(-c4ccccc4)n2)c2c4ccccc4n(c4cccc5c6ccccc6n3c54)c12]

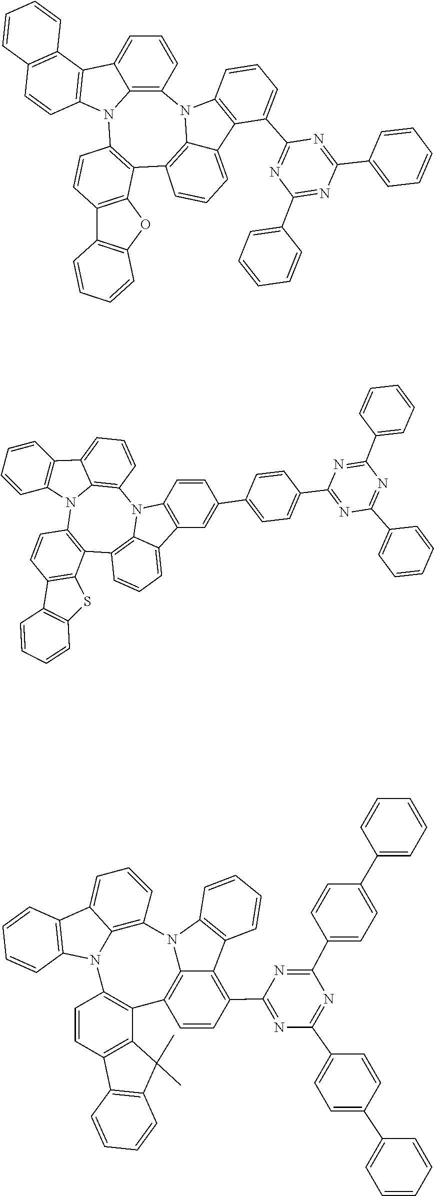 [SMILES: c1ccc(-c2nc(-c3ccccc3)nc(-c3cccc4c3c3cccc5c6c7oc8ccccc8c7ccc6n6c7ccc8ccccc8c7c7cccc(c76)n4c35)n2)cc1 ; c1ccc(-c2nc(-c3ccccc3)nc(-c3ccc(-c4ccc5c(c4)c4cccc6c7c8sc9ccccc9c8ccc7n7c8ccccc8c8cccc(c87)n5c46)cc3)n2)cc1 ; CC1(C)c2ccccc2-c2ccc3c(c21)c1ccc(-c2nc(-c4ccc(-c5ccccc5)cc4)nc(-c4ccc(-c5ccccc5)cc4)n2)c2c4ccccc4n(c4cccc5c6ccccc6n3c54)c12]

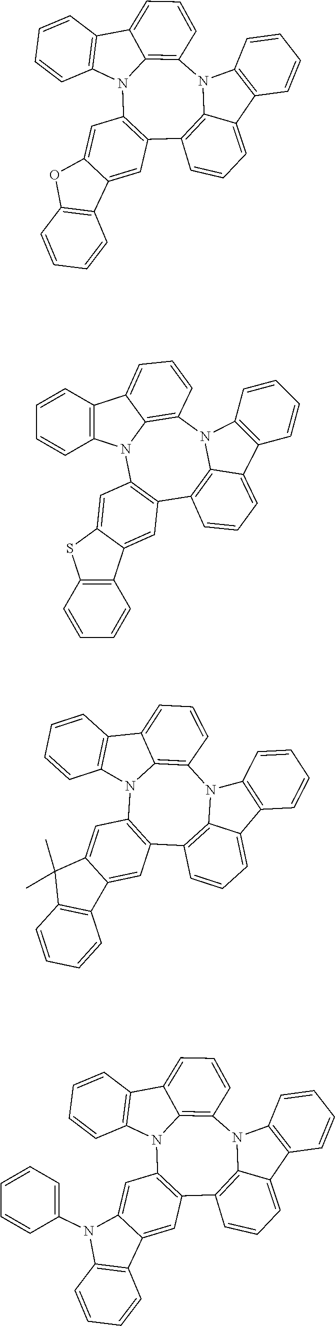 [SMILES: c1ccc2c(c1)oc1cc3c(cc12)c1cccc2c4ccccc4n(c4cccc5c6ccccc6n3c54)c21 ; c1ccc2c(c1)sc1cc3c(cc12)c1cccc2c4ccccc4n(c4cccc5c6ccccc6n3c54)c21 ; CC1(C)c2ccccc2-c2cc3c4cccc5c6ccccc6n(c6cccc7c8ccccc8n(c3cc21)c76)c54 ; c1ccc(-n2c3ccccc3c3cc4c5cccc6c7ccccc7n(c7cccc8c9ccccc9n(c4cc32)c87)c65)cc1]

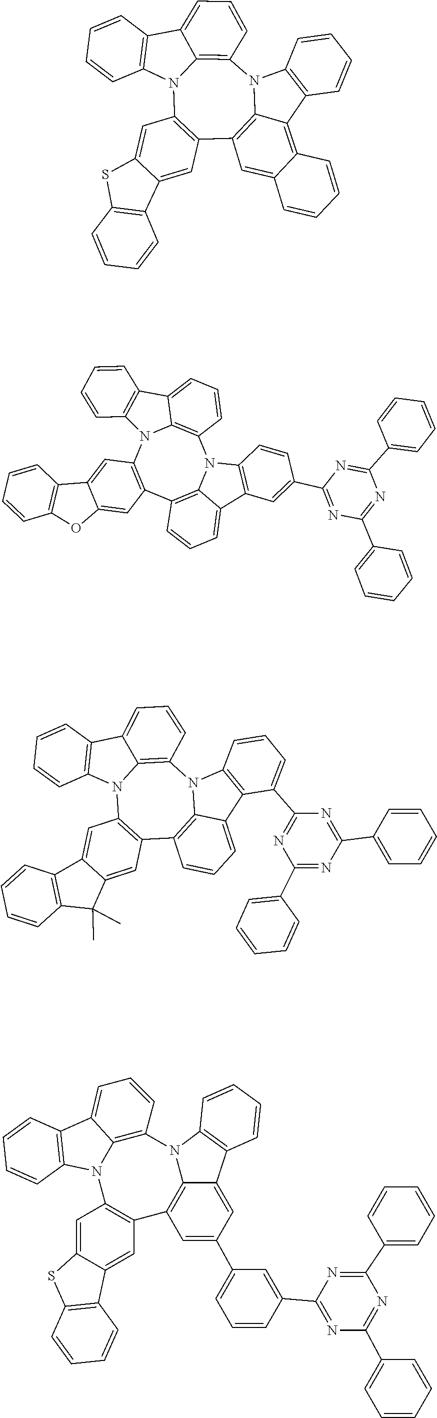 [SMILES: c1ccc2c(c1)cc1c3cc4c(cc3n3c5ccccc5c5cccc(c53)n3c5ccccc5c2c13)sc1ccccc14 ; c1ccc(-c2nc(-c3ccccc3)nc(-c3ccc4c(c3)c3cccc5c6cc7oc8ccccc8c7cc6n6c7ccccc7c7cccc(c76)n4c35)n2)cc1 ; CC1(C)c2ccccc2-c2cc3c(cc21)c1cccc2c4c(-c5nc(-c6ccccc6)nc(-c6ccccc6)n5)cccc4n(c4cccc5c6ccccc6n3c54)c12 ; c1ccc(-c2nc(-c3ccccc3)nc(-c3cccc(-c4cc5c6ccccc6n6c7cccc8c9ccccc9n(c9cc%10sc%11ccccc%11c%10cc9c(c4)c56)c87)c3)n2)cc1]

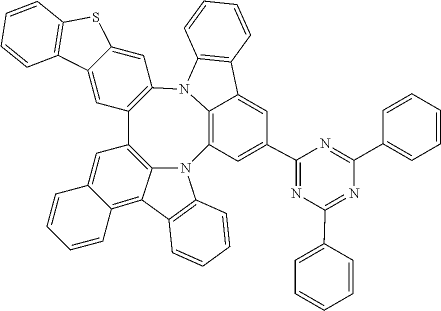 [SMILES: c1ccc(-c2nc(-c3ccccc3)nc(-c3cc4c5ccccc5n5c6cc7sc8ccccc8c7cc6c6cc7ccccc7c7c8ccccc8n(c(c3)c45)c67)n2)cc1]

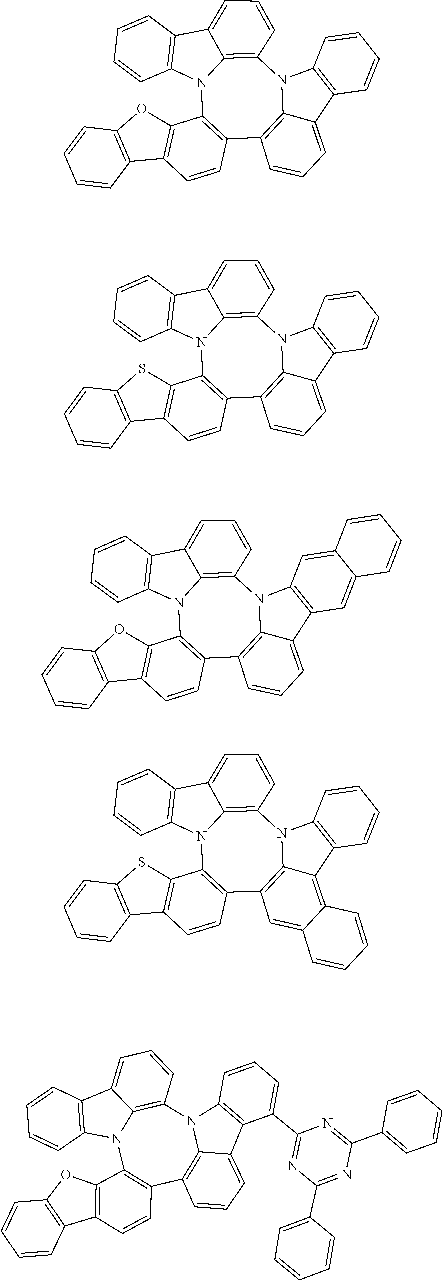 [SMILES: c1ccc2c(c1)oc1c2ccc2c3cccc4c5ccccc5n(c5cccc6c7ccccc7n(c21)c65)c43 ; c1ccc2c(c1)sc1c2ccc2c3cccc4c5ccccc5n(c5cccc6c7ccccc7n(c21)c65)c43 ; c1ccc2cc3c(cc2c1)c1cccc2c4ccc5c6ccccc6oc5c4n4c5ccccc5c5cccc(c54)n3c12 ; c1ccc2c(c1)cc1c3ccc4c5ccccc5sc4c3n3c4ccccc4c4cccc(c43)n3c4ccccc4c2c13 ; c1ccc(-c2nc(-c3ccccc3)nc(-c3cccc4c3c3cccc5c6ccc7c8ccccc8oc7c6n6c7ccccc7c7cccc(c76)n4c53)n2)cc1]

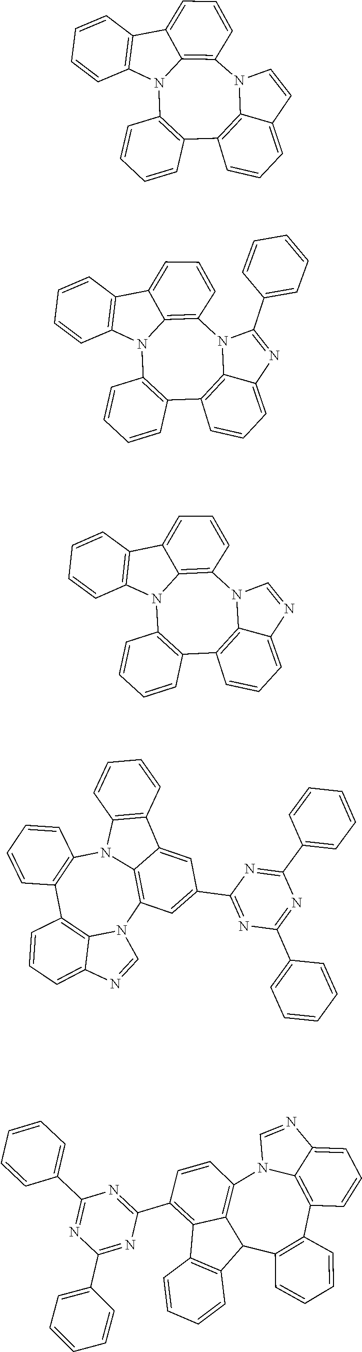 [SMILES: c1cc2ccn3c4cccc5c6ccccc6n(c6ccccc6c(c1)c23)c54 ; c1ccc(-c2nc3cccc4c5ccccc5n5c6ccccc6c6cccc(c65)n2c34)cc1 ; c1cc2ncn3c4cccc5c6ccccc6n(c6ccccc6c(c1)c23)c54 ; c1ccc(-c2nc(-c3ccccc3)nc(-c3cc4c5ccccc5n5c6ccccc6c6cccc7ncn(c(c3)c45)c76)n2)cc1 ; c1ccc(-c2nc(-c3ccccc3)nc(-c3ccc4c5c3-c3ccccc3C5c3ccccc3-c3cccc5ncn-4c35)n2)cc1]

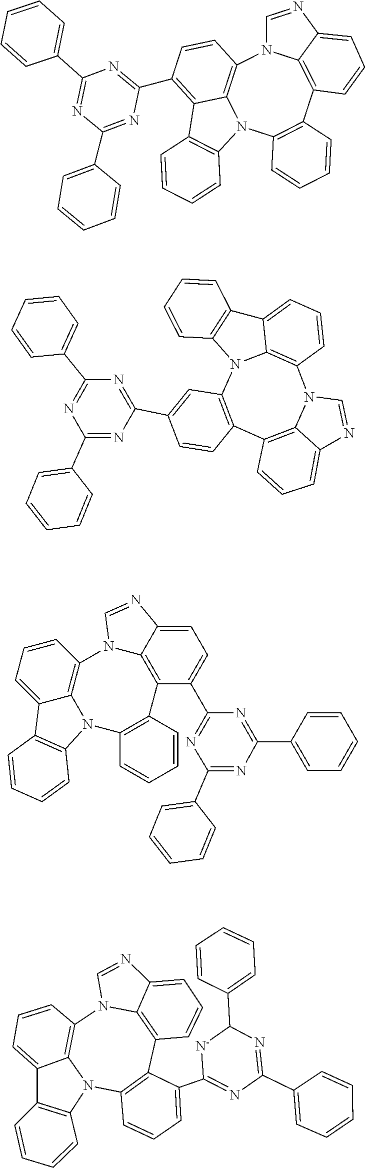 [SMILES: c1ccc(-c2nc(-c3ccccc3)nc(-c3ccc4c5c3c3ccccc3n5c3ccccc3c3cccc5ncn4c53)n2)cc1 ; c1ccc(-c2nc(-c3ccccc3)nc(-c3ccc4c5cccc6ncn(c7cccc8c9ccccc9n(c4c3)c87)c65)n2)cc1 ; c1ccc(-c2nc(-c3ccccc3)nc(-c3ccc4ncn5c6cccc7c8ccccc8n(c8ccccc8c3c45)c76)n2)cc1 ; c1ccc(C2=NC(c3ccccc3)[N-]C(c3cccc4c3c3cccc5ncn(c6cccc7c8ccccc8n4c76)c53)=N2)cc1]

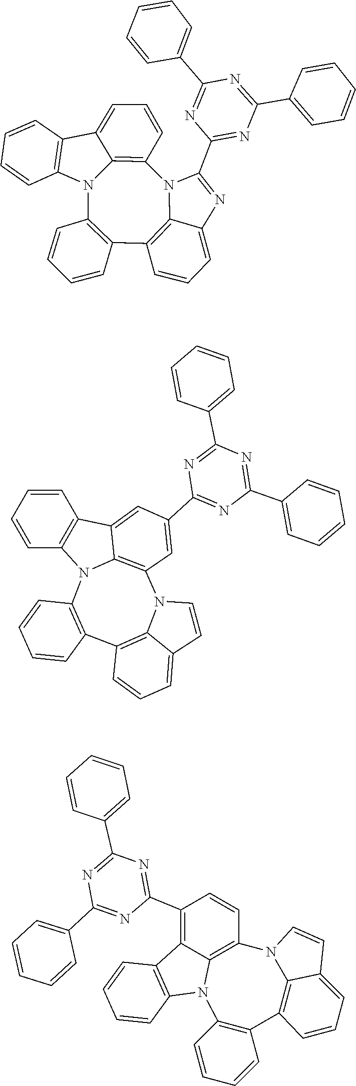 [SMILES: c1ccc(-c2nc(-c3ccccc3)nc(-c3nc4cccc5c6ccccc6n6c7ccccc7c7cccc(c76)n3c45)n2)cc1 ; c1ccc(-c2nc(-c3ccccc3)nc(-c3cc4c5ccccc5n5c6ccccc6c6cccc7ccn(c(c3)c45)c76)n2)cc1 ; c1ccc(-c2nc(-c3ccccc3)nc(-c3ccc4c5c3c3ccccc3n5c3ccccc3c3cccc5ccn4c53)n2)cc1]

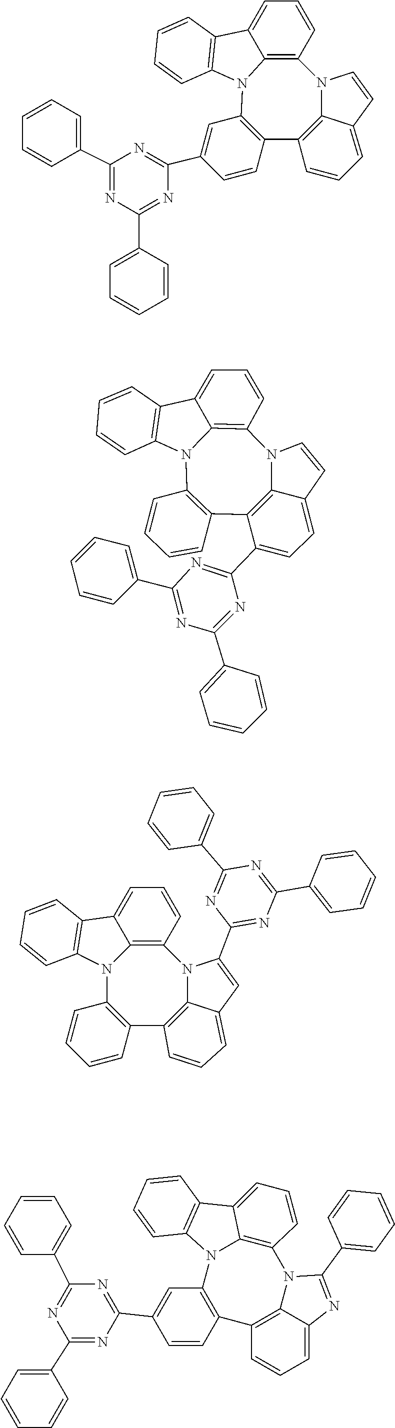 [SMILES: c1ccc(-c2nc(-c3ccccc3)nc(-c3ccc4c5cccc6ccn(c7cccc8c9ccccc9n(c4c3)c87)c65)n2)cc1 ; c1ccc(-c2nc(-c3ccccc3)nc(-c3ccc4ccn5c6cccc7c8ccccc8n(c8ccccc8c3c45)c76)n2)cc1 ; c1ccc(-c2nc(-c3ccccc3)nc(-c3cc4cccc5c6ccccc6n6c7ccccc7c7cccc(c76)n3c45)n2)cc1 ; c1ccc(-c2nc(-c3ccccc3)nc(-c3ccc4c5cccc6nc(-c7ccccc7)n(c7cccc8c9ccccc9n(c4c3)c87)c65)n2)cc1]

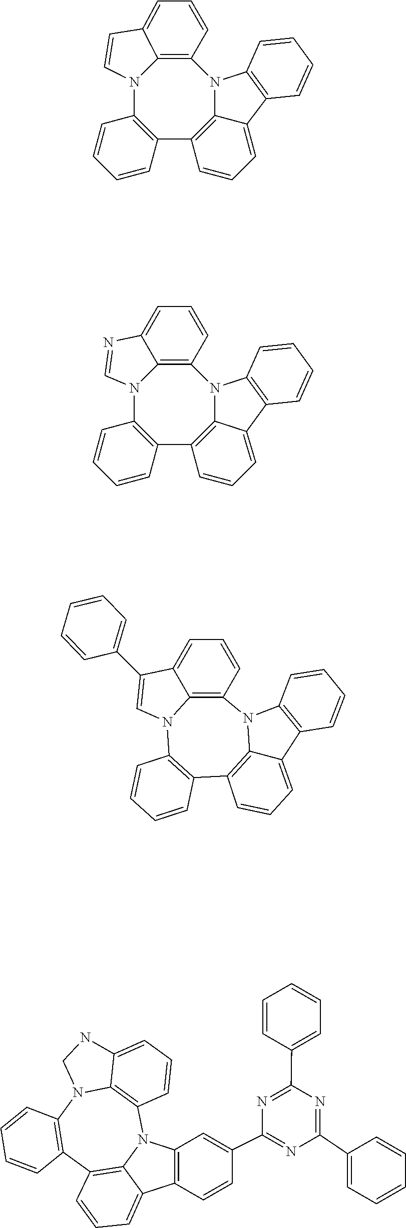

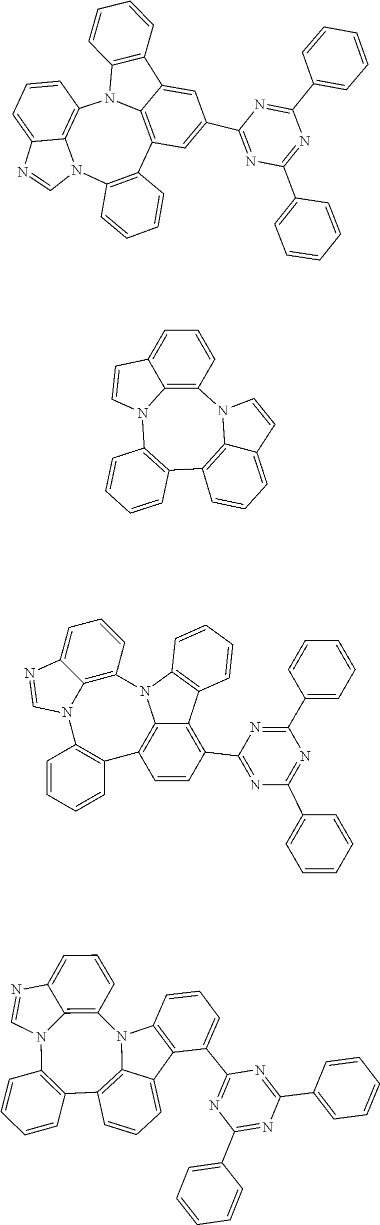 [SMILES: c1ccc(-c2nc(-c3ccccc3)nc(-c3cc4c5ccccc5n5cnc6cccc(c65)n5c6ccccc6c(c3)c45)n2)cc1 ; c1ccc2c(c1)c1cccc3ccn(c4cccc5ccn2c54)c31 ; c1ccc(-c2nc(-c3ccccc3)nc(-c3ccc4c5ccccc5n5cnc6cccc(c65)n5c6ccccc6c3c45)n2)cc1 ; c1ccc(-c2nc(-c3ccccc3)nc(-c3cccc4c3c3cccc5c6ccccc6n6cnc7cccc(c76)n4c53)n2)cc1]

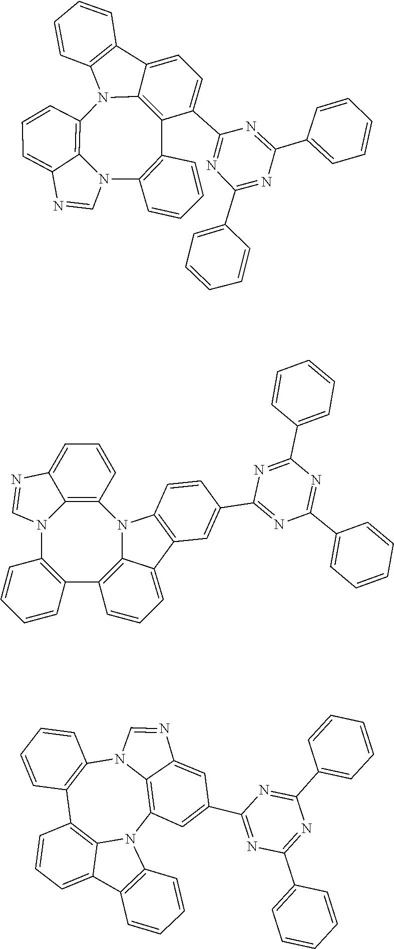 [SMILES: c1ccc(-c2nc(-c3ccccc3)nc(-c3ccc4c5ccccc5n5c6cccc7ncn(c8ccccc8c3c45)c76)n2)cc1 ; c1ccc(-c2nc(-c3ccccc3)nc(-c3ccc4c(c3)c3cccc5c6ccccc6n6cnc7cccc(c76)n4c53)n2)cc1 ; c1ccc(-c2nc(-c3ccccc3)nc(-c3cc4ncn5c6ccccc6c6cccc7c8ccccc8n(c(c3)c45)c67)n2)cc1]

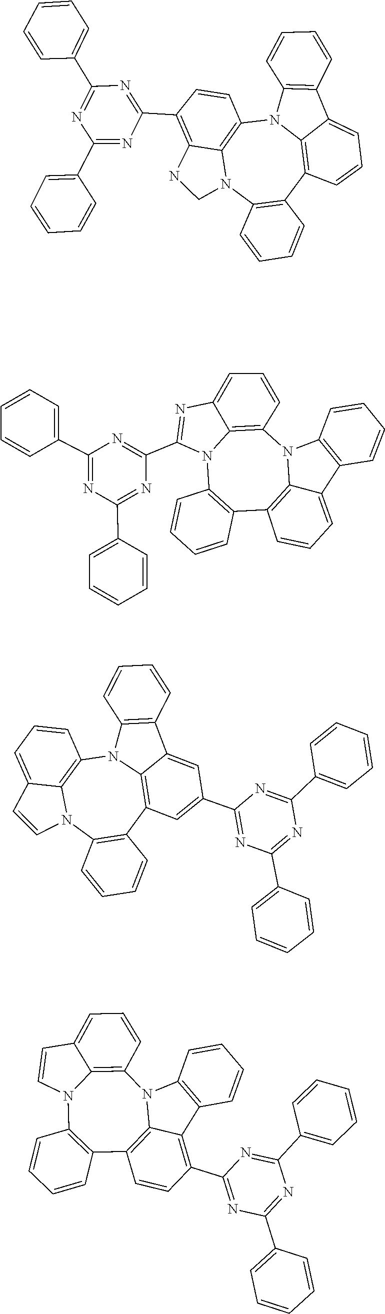 [SMILES: c1ccc(-c2nc(-c3ccccc3)nc(-c3ccc4c5c3NCn5c3ccccc3c3cccc5c6ccccc6n4c35)n2)cc1 ; c1ccc(-c2nc(-c3ccccc3)nc(-c3nc4cccc5c4n3c3ccccc3c3cccc4c6ccccc6n5c34)n2)cc1 ; c1ccc(-c2nc(-c3ccccc3)nc(-c3cc4c5ccccc5n5ccc6cccc(c65)n5c6ccccc6c(c3)c45)n2)cc1 ; c1ccc(-c2nc(-c3ccccc3)nc(-c3ccc4c5ccccc5n5ccc6cccc(c65)n5c6ccccc6c3c45)n2)cc1]

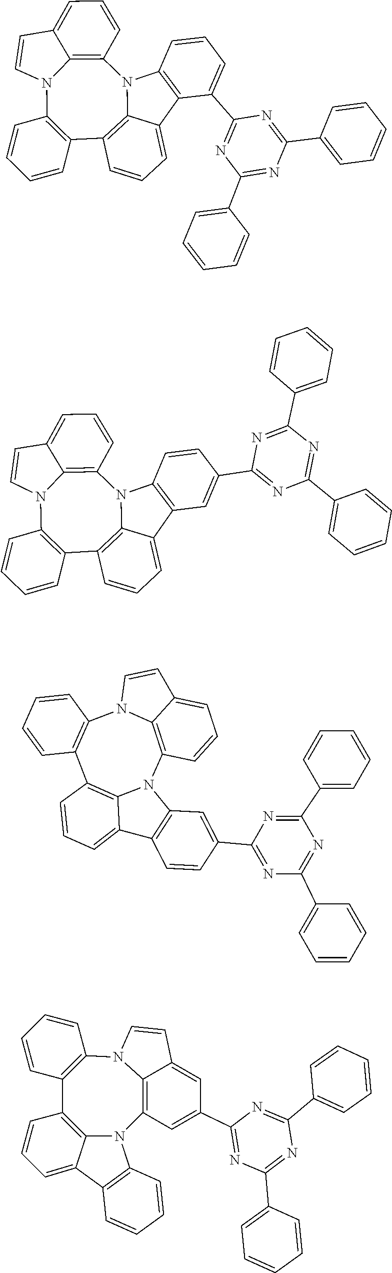 [SMILES: c1ccc(-c2nc(-c3ccccc3)nc(-c3cccc4c3c3cccc5c6ccccc6n6ccc7cccc(c76)n4c53)n2)cc1 ; c1ccc(-c2nc(-c3ccccc3)nc(-c3ccc4c(c3)c3cccc5c6ccccc6n6ccc7cccc(c76)n4c53)n2)cc1 ; c1ccc(-c2nc(-c3ccccc3)nc(-c3ccc4c5cccc6c7ccccc7n7ccc8cccc(c87)n(c4c3)c65)n2)cc1 ; c1ccc(-c2nc(-c3ccccc3)nc(-c3cc4ccn5c6ccccc6c6cccc7c8ccccc8n(c(c3)c45)c67)n2)cc1]

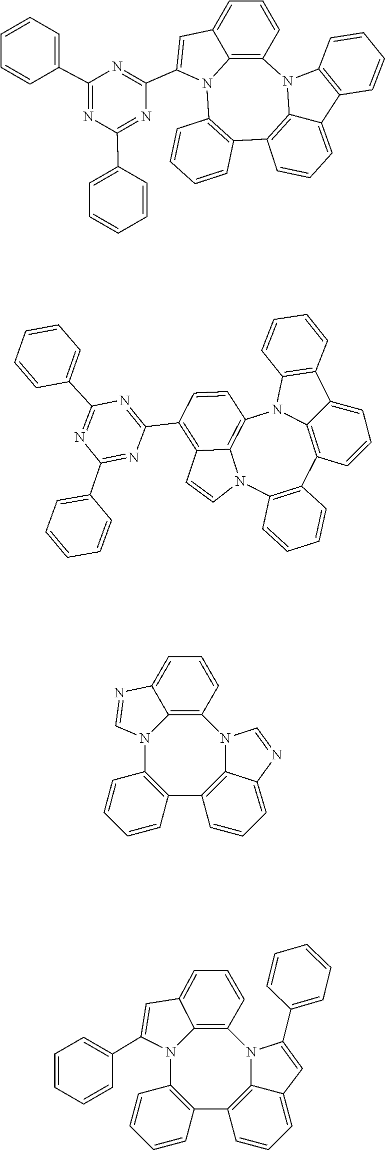 [SMILES: c1ccc(-c2nc(-c3ccccc3)nc(-c3cc4cccc5c4n3c3ccccc3c3cccc4c6ccccc6n5c34)n2)cc1 ; c1ccc(-c2nc(-c3ccccc3)nc(-c3ccc4c5c3ccn5c3ccccc3c3cccc5c6ccccc6n4c35)n2)cc1 ; c1ccc2c(c1)c1cccc3ncn(c4cccc5ncn2c54)c31 ; c1ccc(-c2cc3cccc4c5ccccc5n5c(-c6ccccc6)cc6cccc(c65)n2c34)cc1]

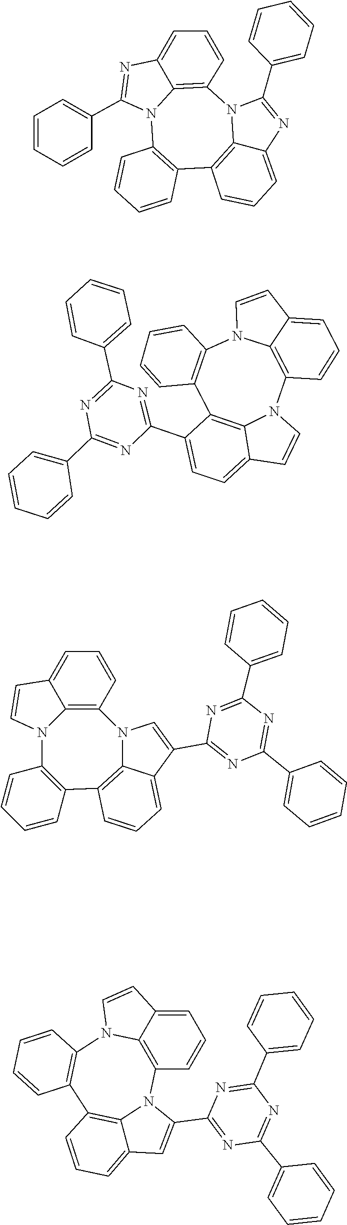 [SMILES: c1ccc(-c2nc3cccc4c5ccccc5n5c(-c6ccccc6)nc6cccc(c65)n2c34)cc1 ; c1ccc(-c2nc(-c3ccccc3)nc(-c3ccc4ccn5c6cccc7ccn(c8ccccc8c3c45)c76)n2)cc1 ; c1ccc(-c2nc(-c3ccccc3)nc(-c3cn4c5cccc6ccn(c7ccccc7c7cccc3c74)c65)n2)cc1 ; c1ccc(-c2nc(-c3ccccc3)nc(-c3cc4cccc5c6ccccc6n6ccc7cccc(c76)n3c45)n2)cc1]

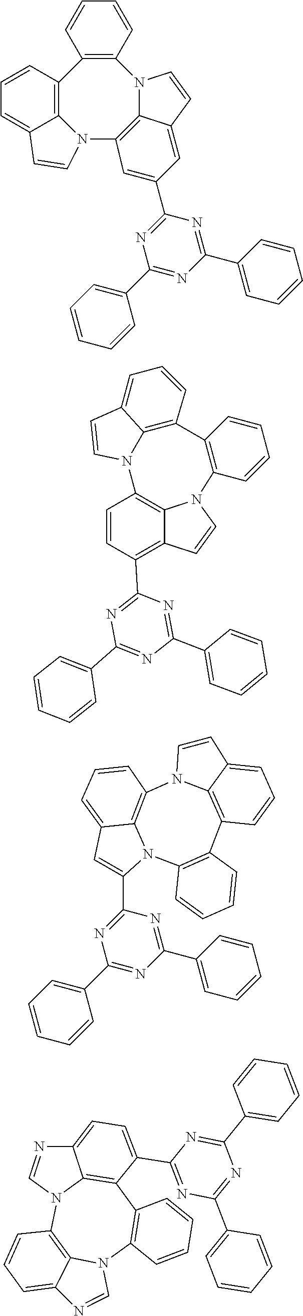

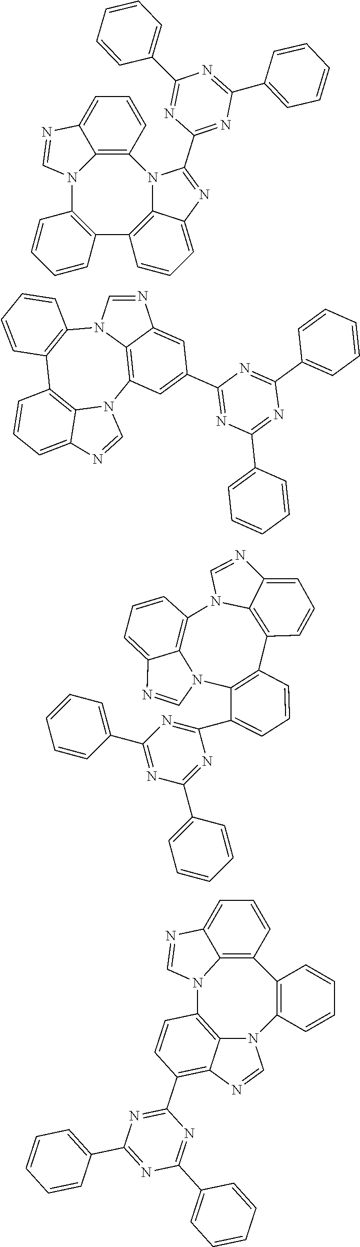

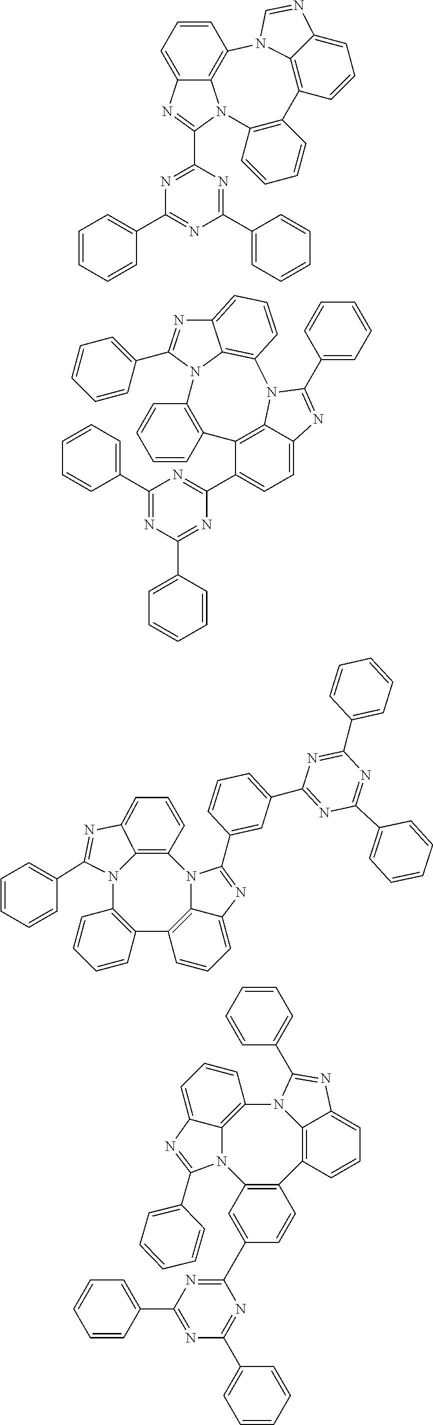

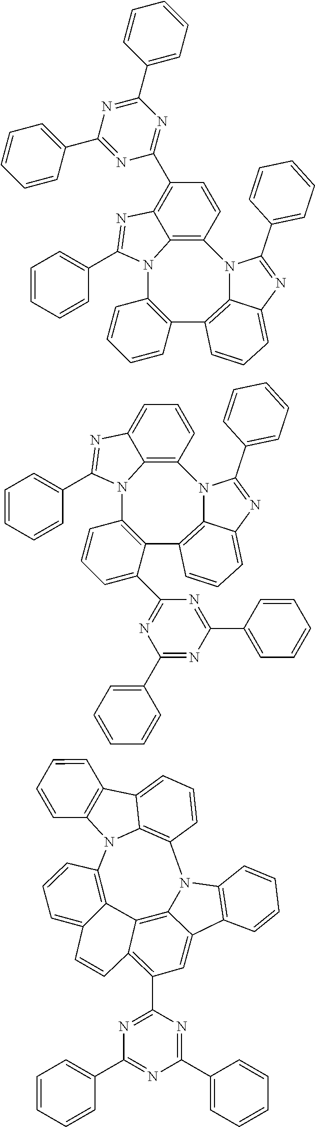

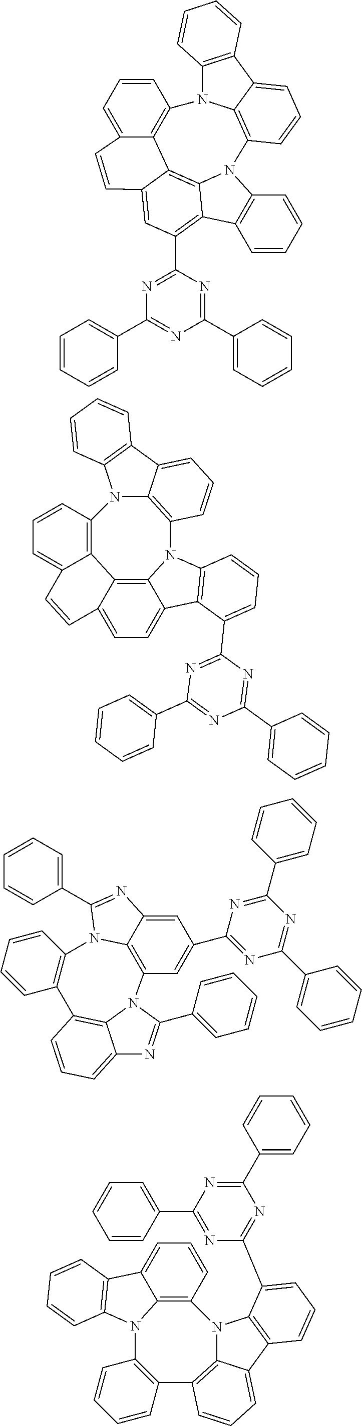

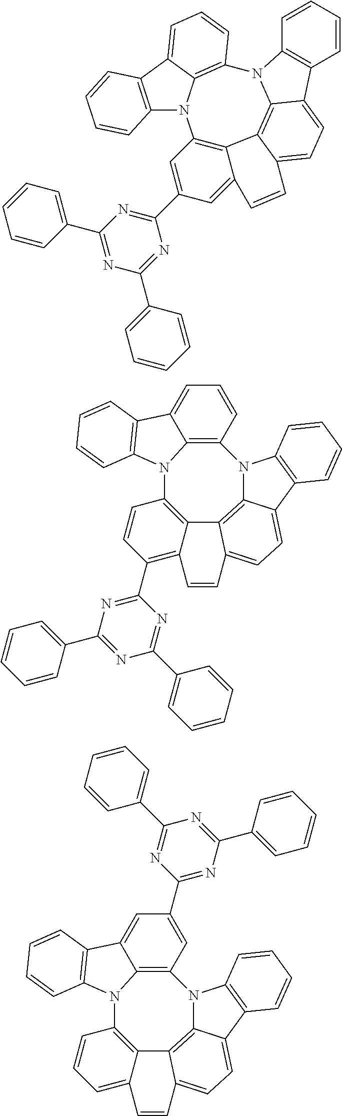

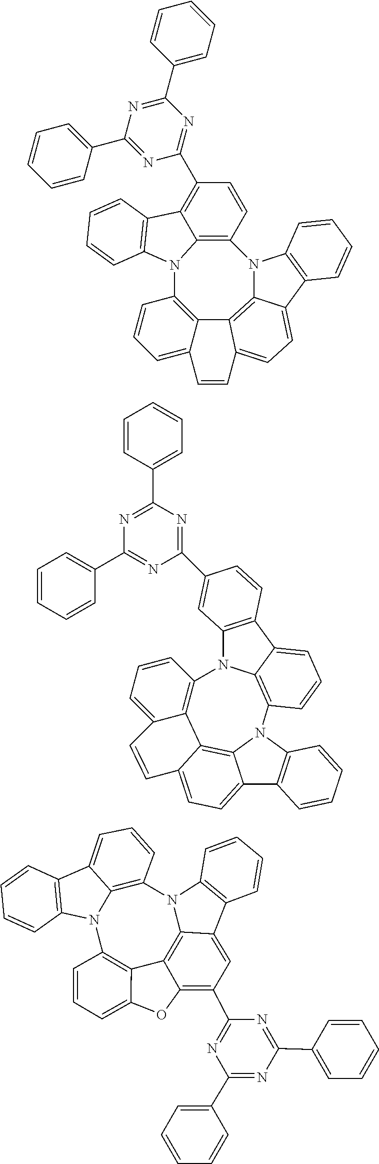

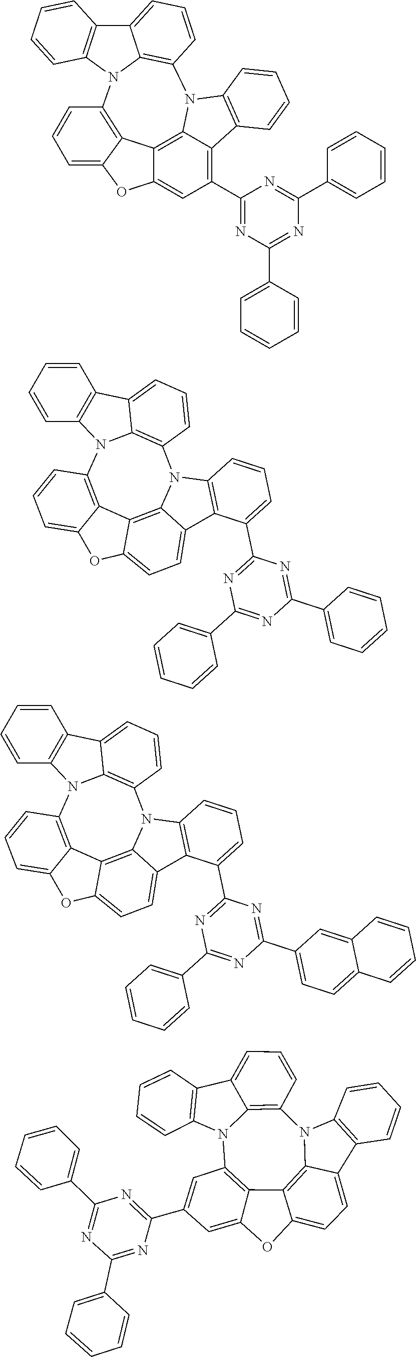

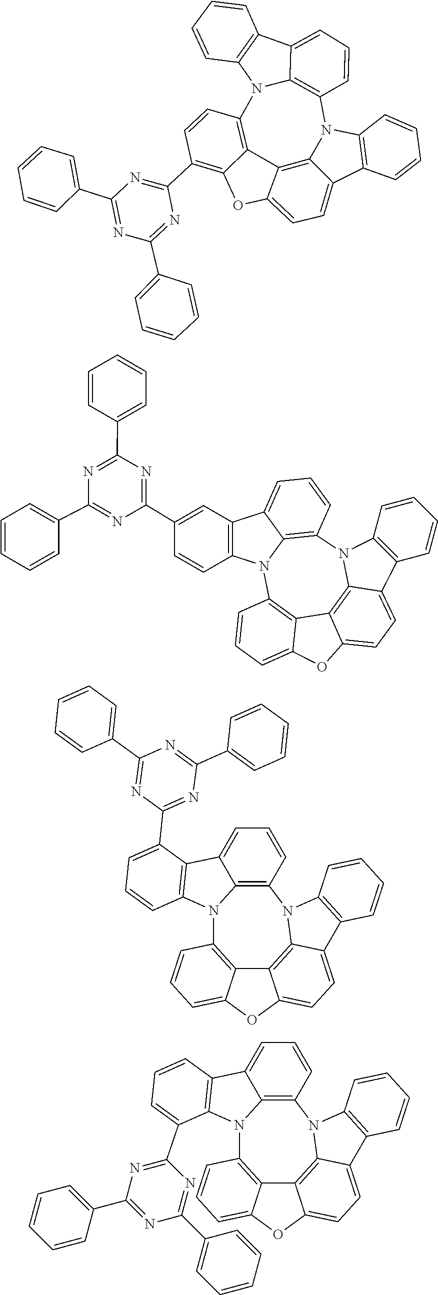

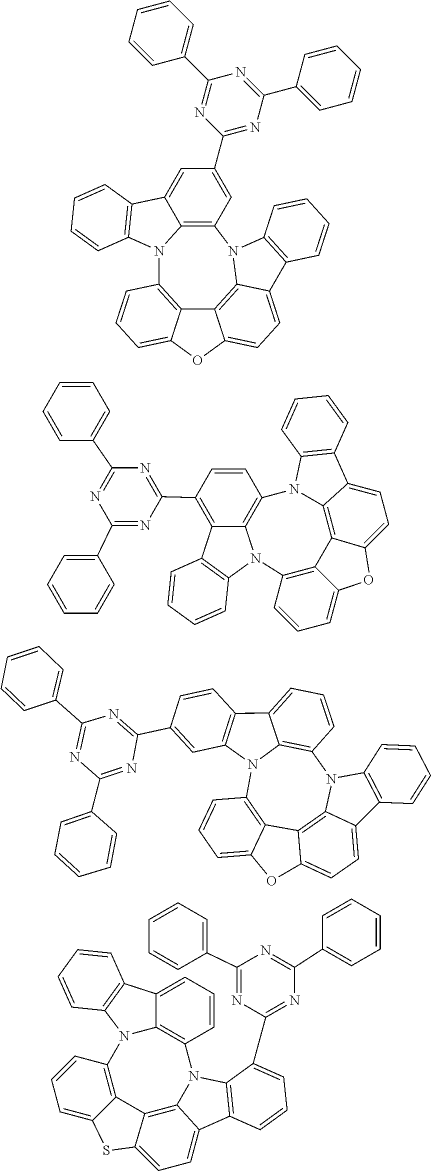

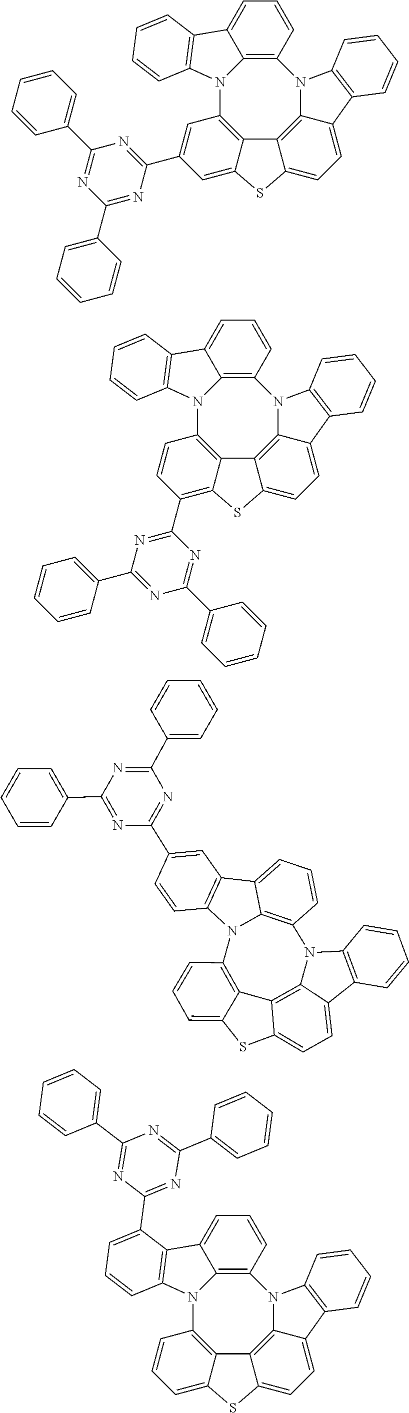

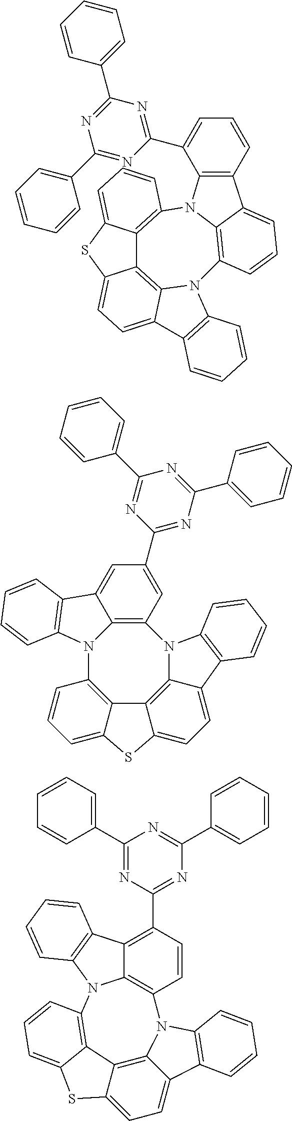

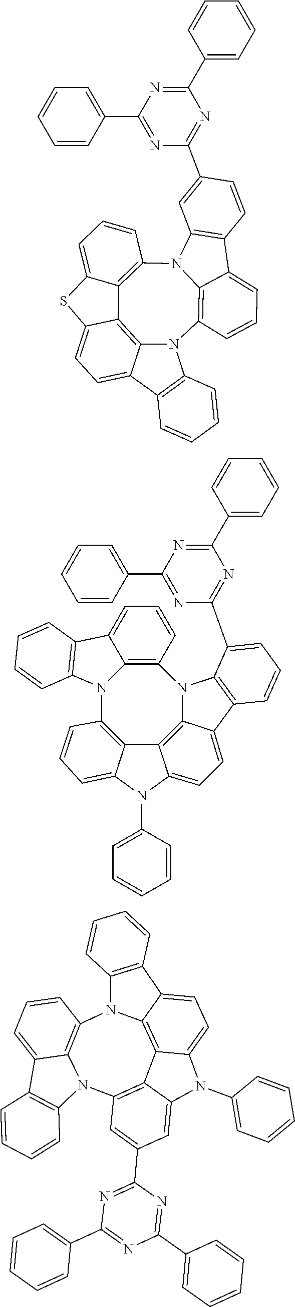

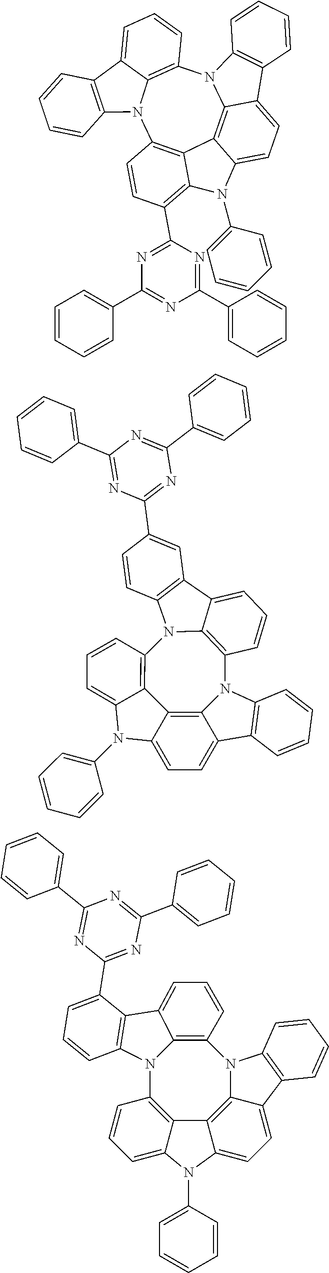

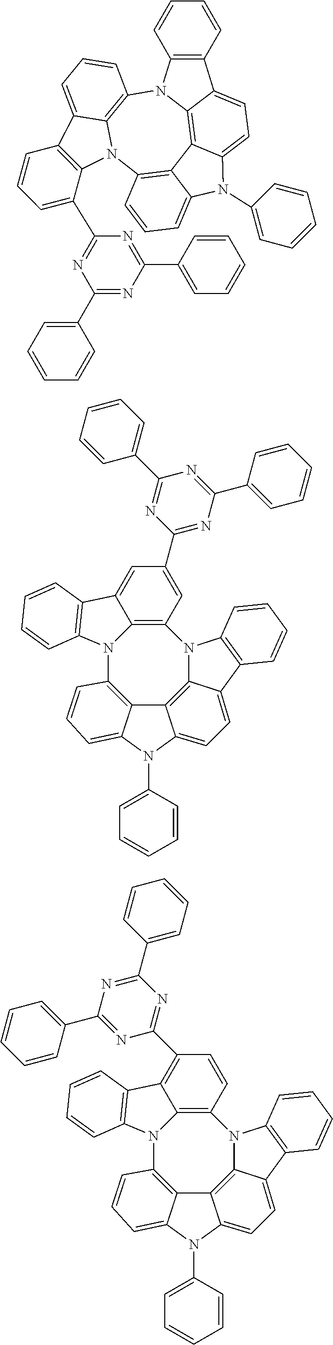

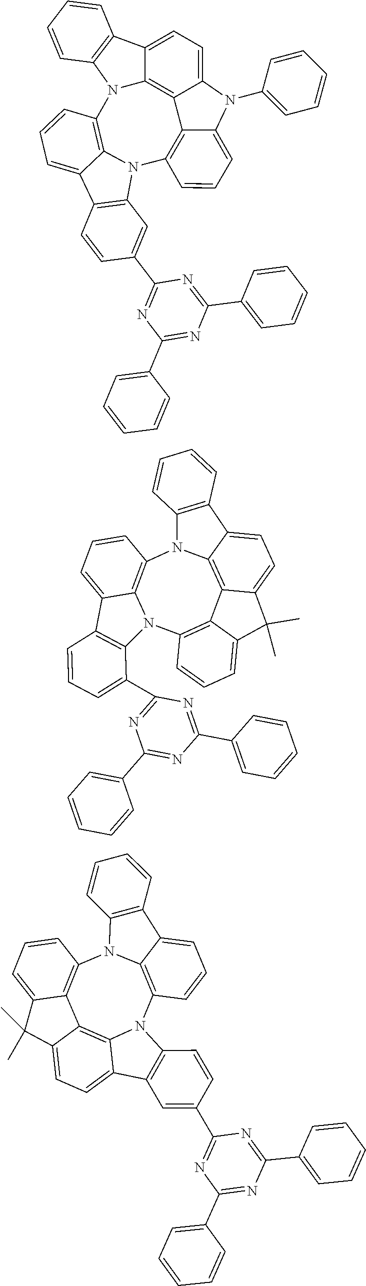

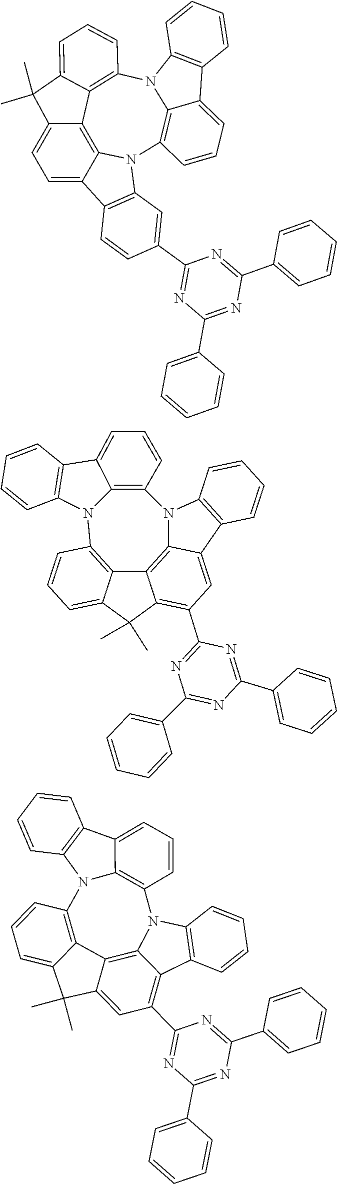

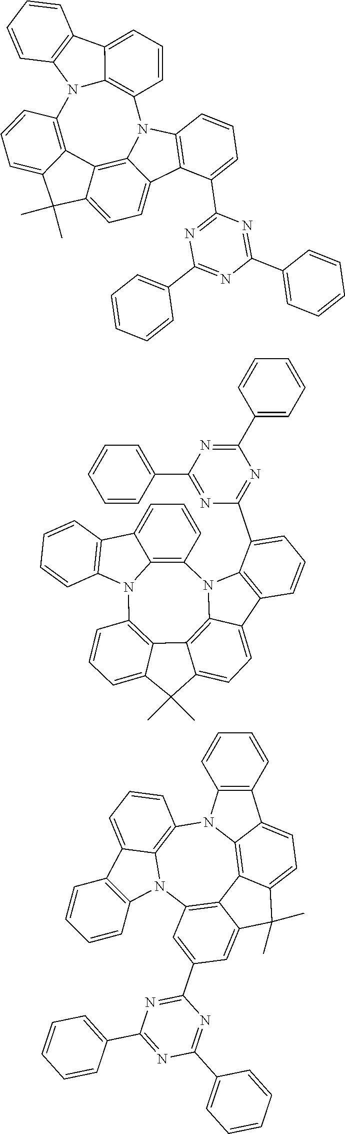

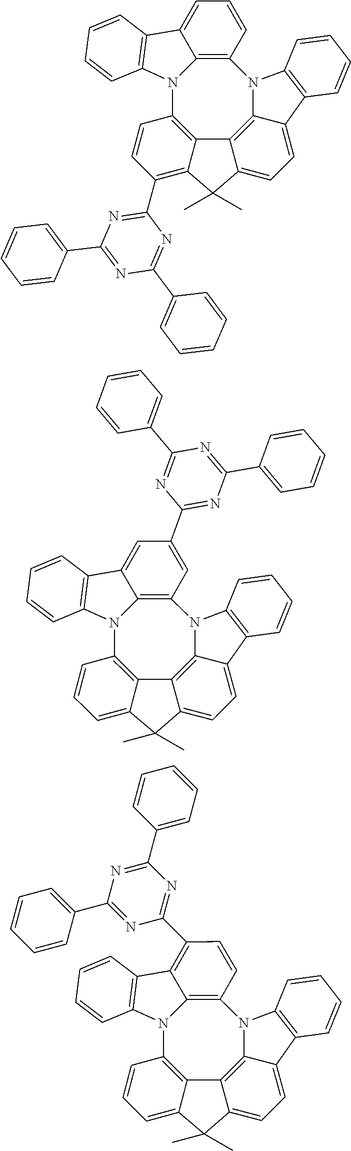

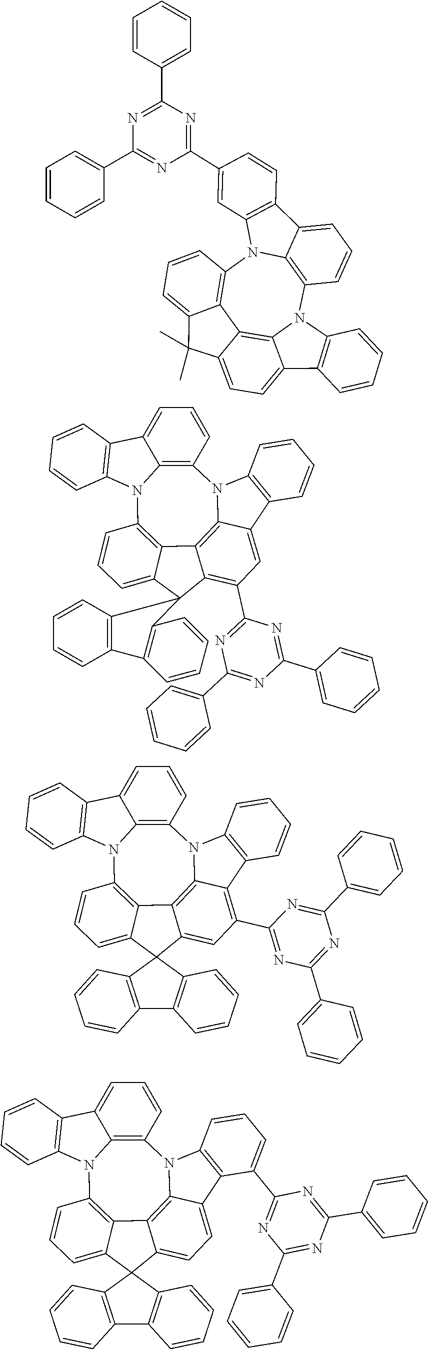

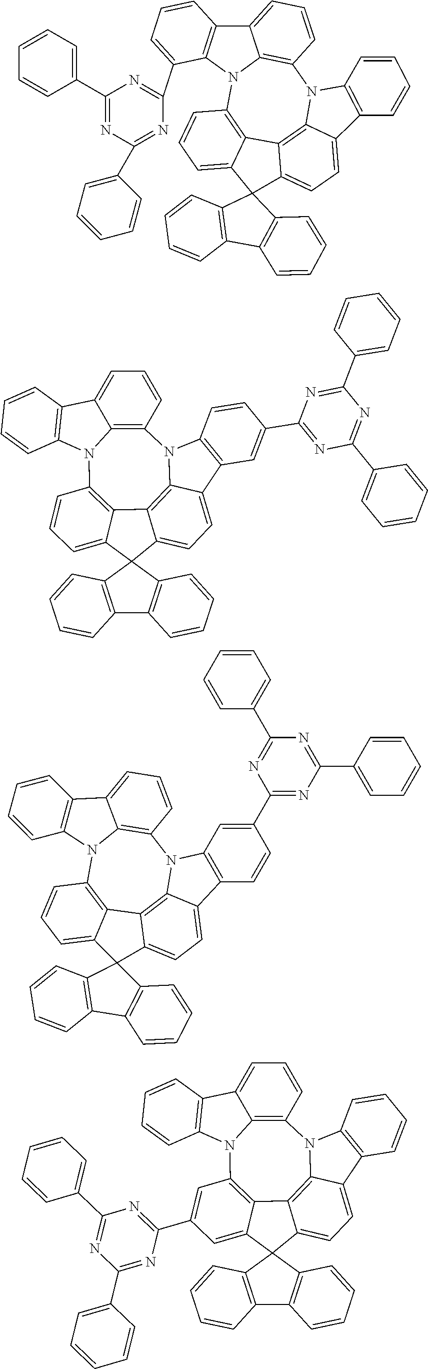

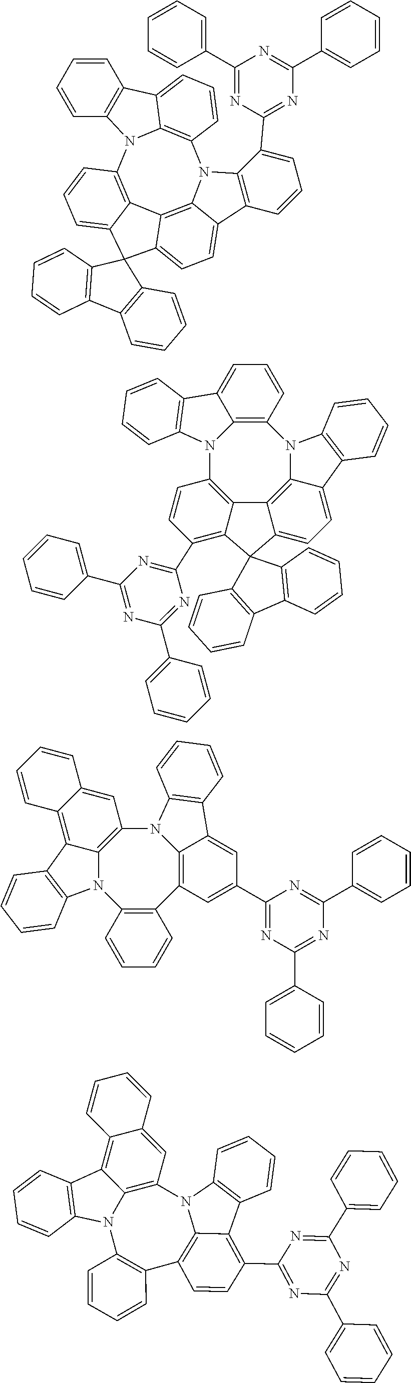

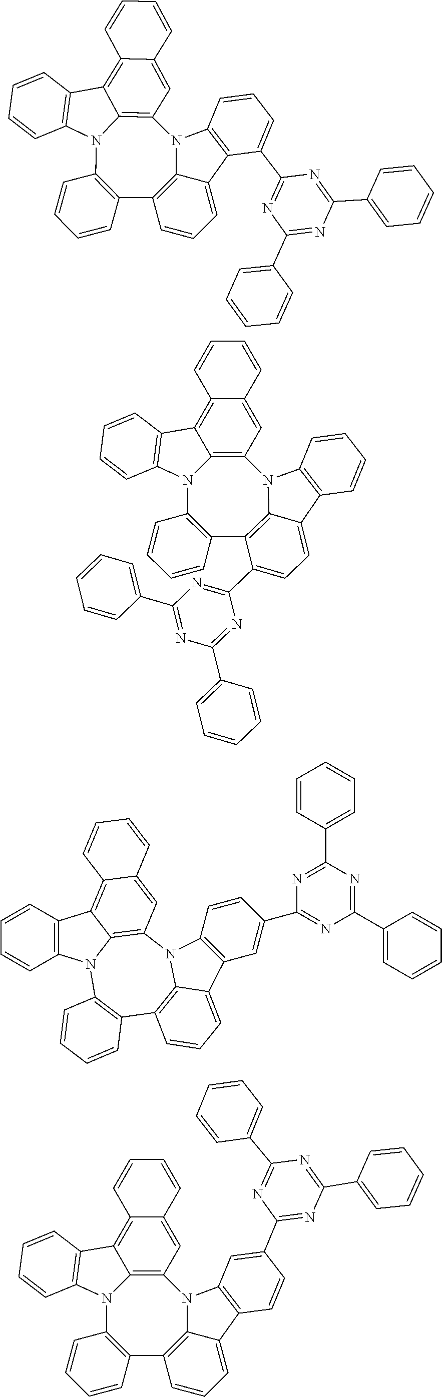

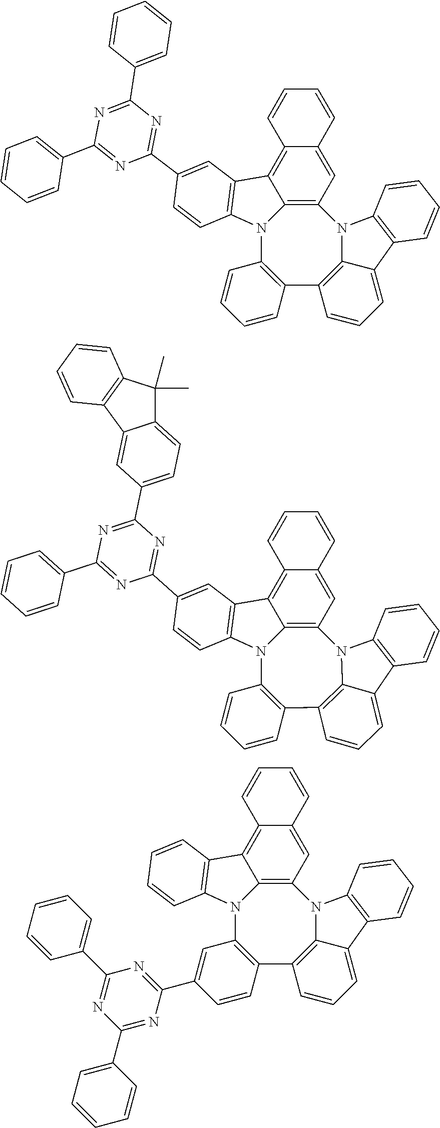

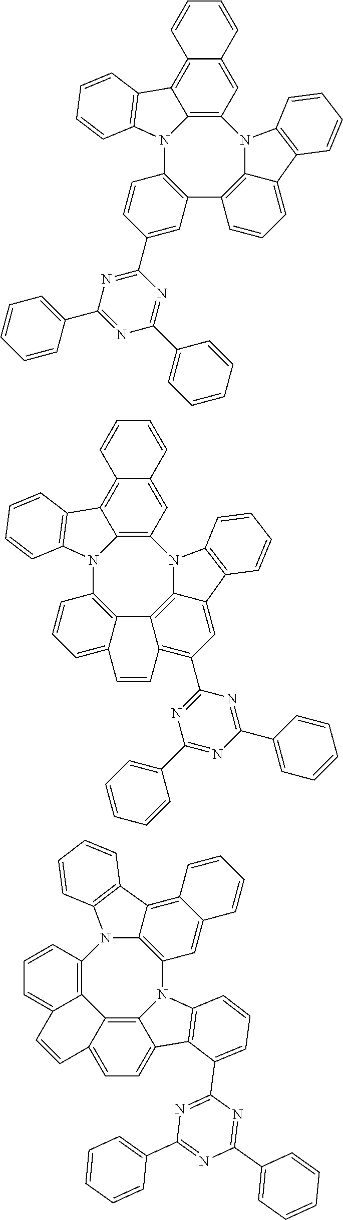

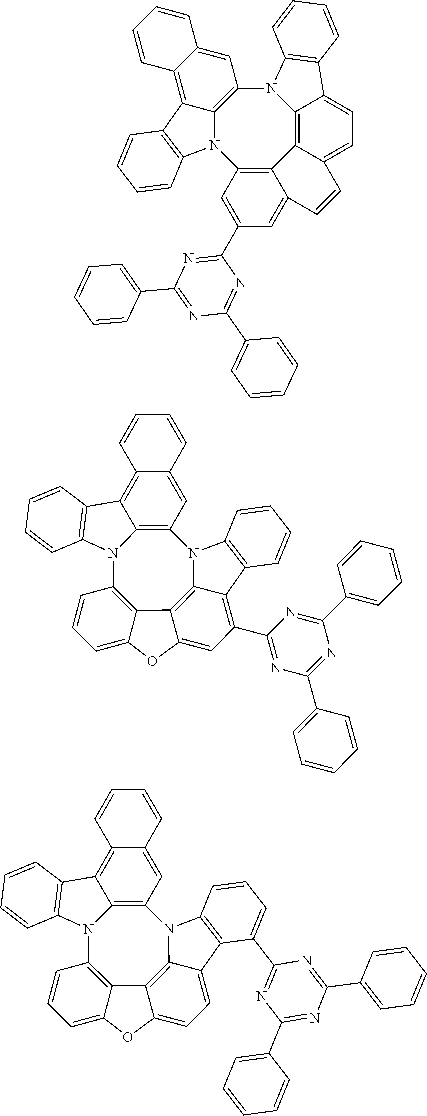 [SMILES: c1ccc(-c2nc(-c3ccccc3)nc(-c3cc4ccc5ccc6c7ccccc7n7c8cc9ccccc9c9c%10ccccc%10n(c(c3)c4c5c67)c98)n2)cc1 ; c1ccc(-c2nc(-c3ccccc3)nc(-c3cc4oc5cccc6c5c4c4c3c3ccccc3n4c3cc4ccccc4c4c5ccccc5n6c43)n2)cc1 ; c1ccc(-c2nc(-c3ccccc3)nc(-c3cccc4c3c3ccc5oc6cccc7c6c5c3n4c3cc4ccccc4c4c5ccccc5n7c43)n2)cc1]

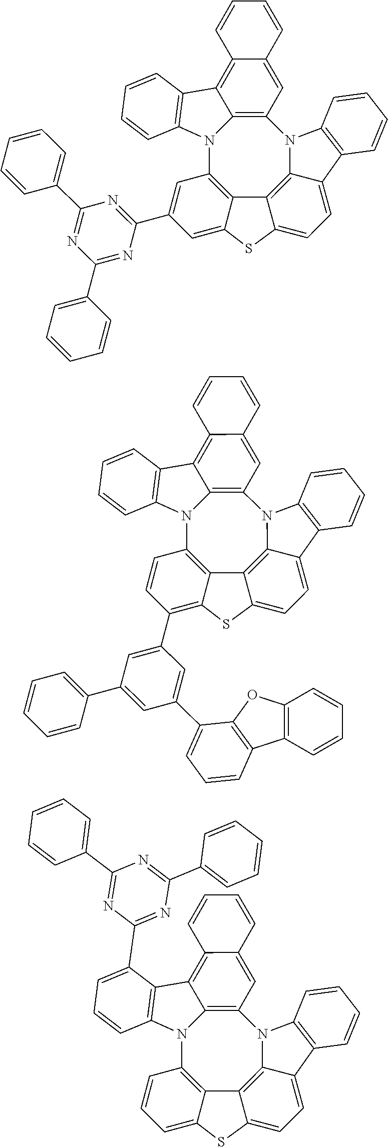

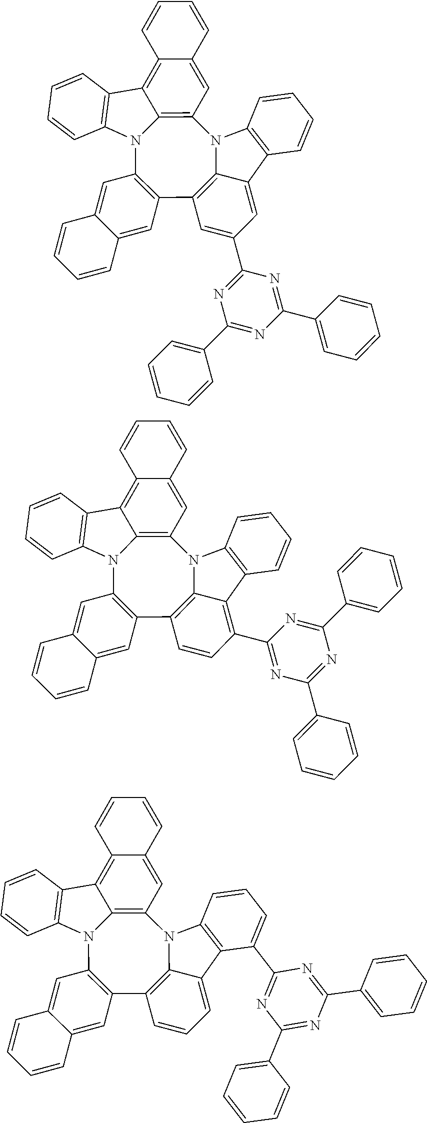

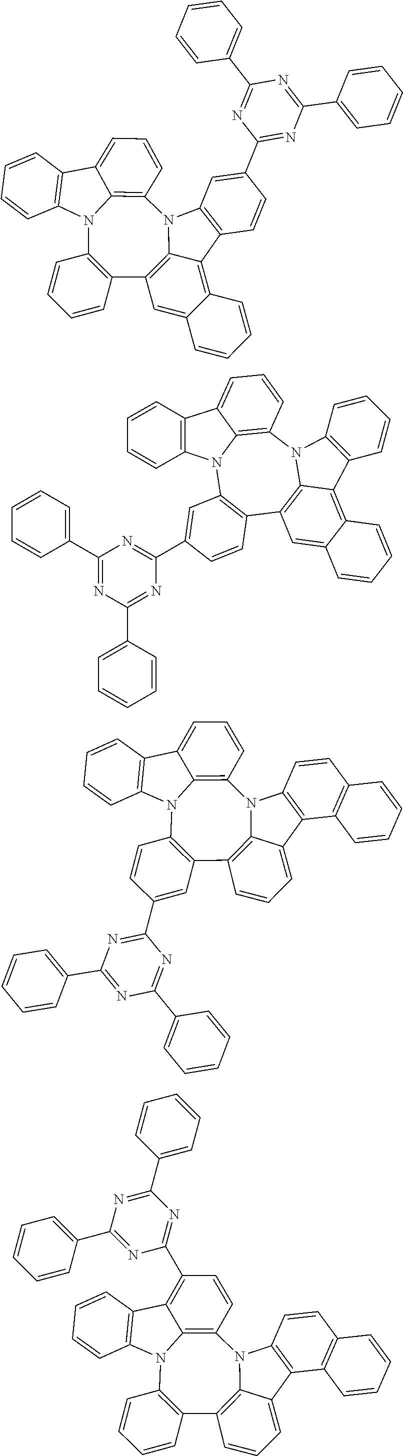

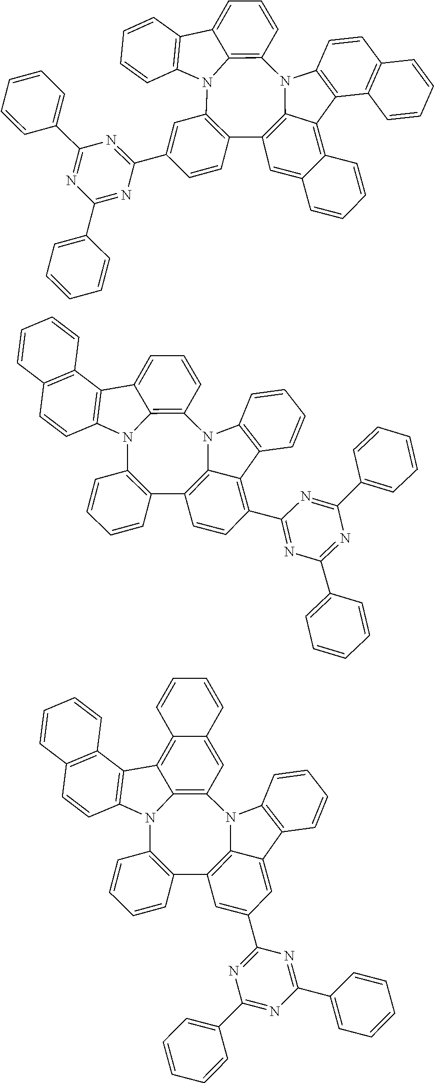

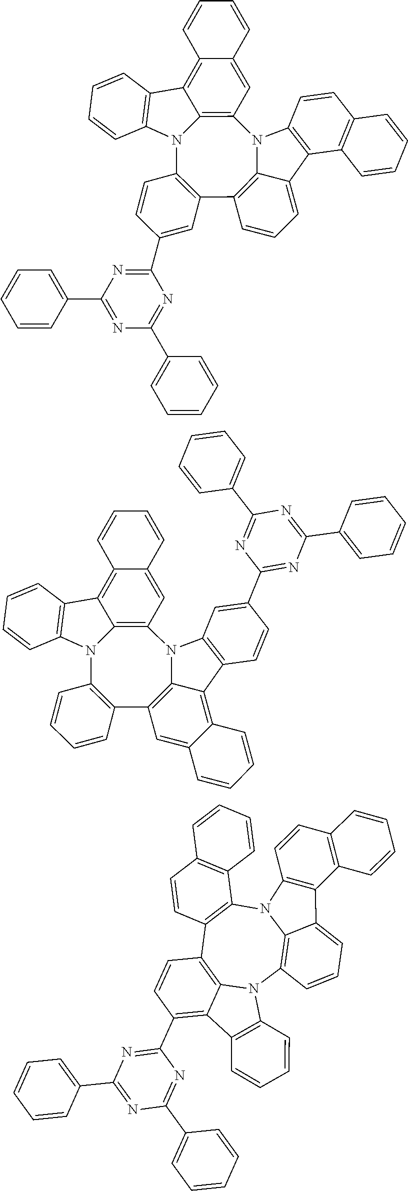

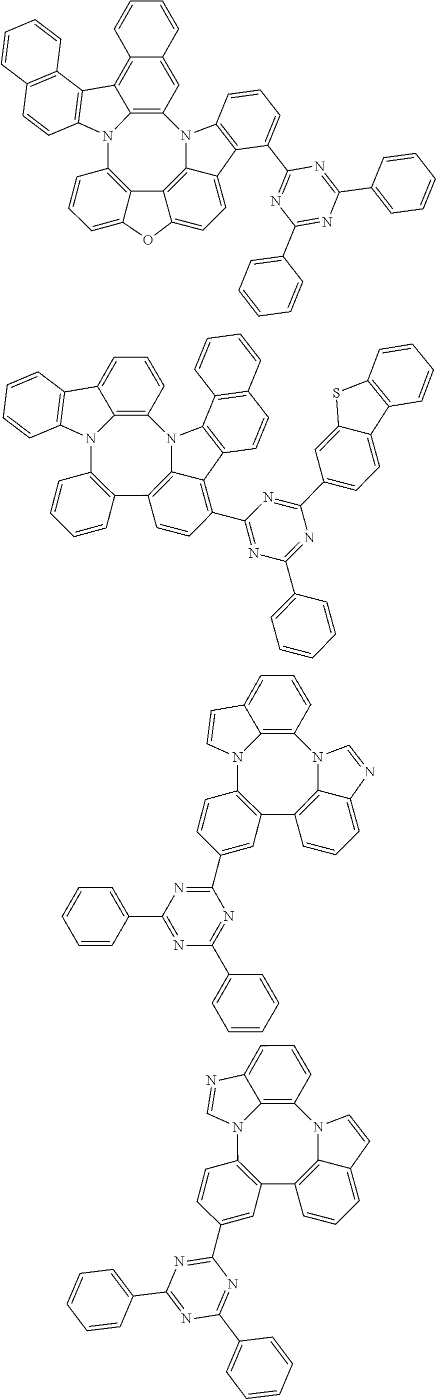

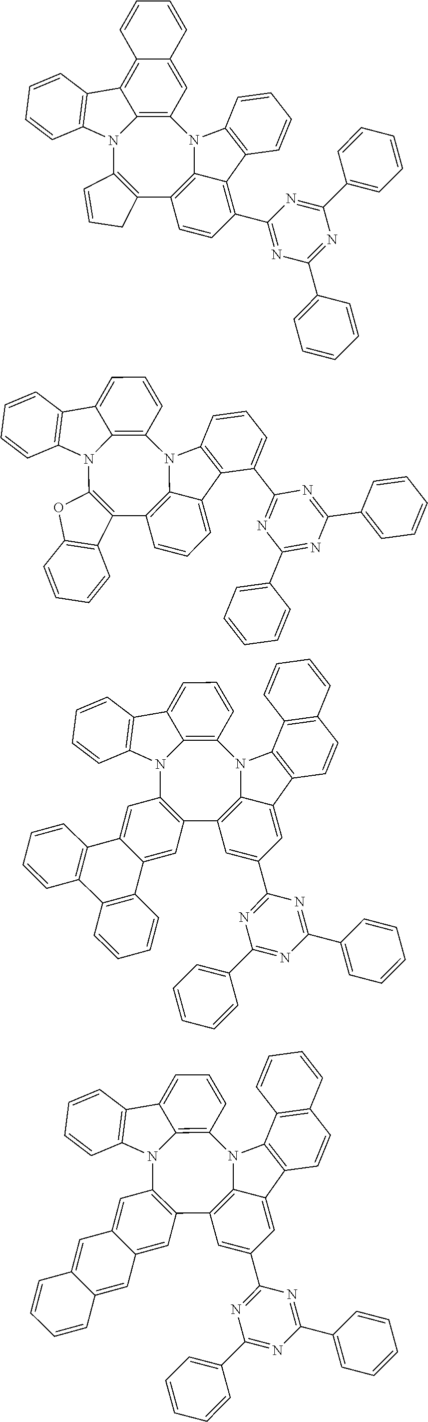

The nitrogen-containing heterocyclic compound of the disclosure can be used as functional materials in electronic devices. The functional materials include, but not limited to, hole injection materials, hole transport materials, electron transport materials, electron injection materials, electron blocking materials (EBMs), hole blocking materials, light-emitting materials, and host materials.

In one embodiment, the nitrogen-containing heterocyclic compound of the disclosure is used as host materials, in particular, phosphorescent host materials.

The phosphorescent host materials need to have an appropriate triplet energy level (E_{T1}). In some embodiments, the compound of the disclosure has the E_{T1} greater than or equal to 2.2 eV, preferably greater than or equal to 2.4 eV, more preferably greater than or equal to 2.6 eV.

In one embodiment, the nitrogen-containing heterocyclic compound of the disclosure needs to have a suitable resonance factor f(S1) to facilitate the transfer of excitons from the host to the guest, so as to improve luminous efficiency of devices. It is preferably to have the f(S1) greater than or equal to 0.01, more preferably greater than or equal to 0.05, most preferably greater than or equal to 0.08.

In another embodiment, the nitrogen-containing heterocyclic compound of the disclosure needs to have a suitable energy level difference ΔE_{ST} between the singlet energy level and the triplet energy level to facilitate the transfer of excitons from the host to the guest, so as to improve luminous efficiency of devices. It is preferably to have the ΔE_{ST} less than or equal to 0.9 eV, more preferably less than or equal to 0.6 eV, most preferably less than or equal to 0.4 eV.

In some embodiments, the nitrogen-containing heterocyclic compound of the disclosure has the luminescence function and has a luminescence wavelength ranging from 300 to 1,000 nm, preferably ranging from 350 to 900 nm, more preferably ranging from 400 to 800 nm. The luminescence here refers to photoluminescence or electroluminescence.

The disclosure further relates to a mixture that includes the organic compound as described above and at least one organic functional material. The organic functional material is selected from a hole injection material, a hole transport material, an electron transport material, an electron injection material, an electron blocking material, a hole blocking material, a light-emitting material, or a host material. The light-emitting material is selected from a singlet emitter, a triplet emitter, or an organic thermally excited delayed fluorescent (TADF) material. Detailed descriptions of these organic functional materials can refer to patent applications WO 2010/135519 A1, US 2009/0134784 A1, and WO 2011/110277 A1.

In one embodiment, the mixture includes at least one organic compound according to the disclosure and one light-emitting material, and the light-emitting material is selected from a singlet emitter, a triplet emitter, or a TADF emitter.

In one embodiment, the mixture includes one organic compound according to the disclosure and another host material. The organic compound according to the disclosure here can be used as a second host having a weight percentage ranging from 30% to 70%.

Detailed description of the singlet emitter, the triplet emitter, the TADF material, and the host material in the disclosure can refer to patent application WO 2018/095390 A1.

The disclosure further relates to a composition that includes at least one organic compound or the mixture as described above, and at least one organic solvent. The at least one organic solvent is selected from an aromatic-based solvent, an heteroaromatic-based solvent, an ester-based solvent, an aromatic ketone-based solvent, an aromatic ether-based solvent, an aliphatic ketone-based solvent, an aliphatic ether-based solvent, an alicyclic compound, an olefin compound, a borate ester compound, a phosphate ester compound, or a mixture of two or more of the solvents above.

For the composition according to the disclosure, a boiling point needs to be considered during selecting the organic solvent. In the disclosure, the boiling point of the organic solvent is greater than or equal to 150° C., preferably greater than or equal to 180° C., more preferably greater than or equal to 200° C., further more preferably greater than or equal to 250° C., most preferably greater than or equal to 300° C. The boiling point within any of these ranges is beneficial to preventing nozzles of inkjet printing heads from clogging. The organic solvent can be evaporated from a solvent system to form a film including the functional material.

In one preferable embodiment, the composition according to the disclosure is a solution.

In another preferable embodiment, the composition according to the disclosure is a suspension.

The composition in the embodiments of the disclosure may include 0.01 to 10 wt % of the organic compound or the mixture according to the disclosure, preferably 0.1 to 5 wt %, more preferably 0.2 to 5 wt %, most preferably 0.25 to 3 wt %.

The disclosure further relates to the use of the composition as a coating or printing ink in the preparation of organic electronic devices, preferably a preparation method of printing or coating.

The disclosure further relates to the uses of the organic compound, the mixture, or the composition in organic electronic devices. The organic electronic devices are selected from, but not limited to, organic light-emitting diodes (OLEDs), organic photovoltaic cells, an organic light-emitting cells, organic field-effect transistors, organic light-emitting field-effect transistors, organic lasers, organic spin electronic devices, organic sensors, organic plasmon emission diodes (OPEDs), or the like, in particular, preferably selected from OLEDs. In the embodiments of the disclosure, the organic compound, the mixture, or the composition is preferably used in light-emitting layers of OLED devices.

The disclosure further relates to an organic electronic device that includes at least one organic compound or the mixture as described above. Further, the organic electronic device includes at least one cathode, at least one anode, and at least one functional layer disposed between the cathode and the anode, and the functional layer includes at least one organic compound or the mixture as described above. The organic electronic device can be any one of the organic electronic devices as described above.

In some preferable embodiments, a light-emitting layer of an electroluminescence device includes one organic compound or the mixture as described above.

In some preferable embodiments, a light-emitting layer of an electroluminescence device includes one organic compound as described above, a combination or mixture of one organic compound as described above and one phosphorescent material, a combination or mixture of one organic compound as described above and one host material, or a combination or mixture of one organic compound as described above and one TADF material.

The above-mentioned light-emitting device, especially the OLED, includes a substrate, an anode, at least one light-emitting layer, and a cathode.

The substrate may be opaque or transparent. A transparent substrate can be used to prepare a transparent light-emitting element. Detailed description about the substrate can refer to Bulovic et al. (Nature 1996, 380, p29), and Gu et al. (Appl Phys Lett 1996, 68, p2606). The substrate may be rigid or elastic. The substrate may be plastic, metal, a semiconductor chip, or glass. It is preferable for the substrate to have a smooth surface. A substrate without surface defects is a particularly ideal choice. In one preferable embodiment, the substrate may be flexible, and selected from, but not limited to, a polymer film or plastic. A glass transition temperature Tg of a material of the substrate is greater than 150° C., preferably greater than 200° C., more preferably greater than 250° C., most preferably greater than 300° C. Suitable examples of the material of a flexible substrate include polyethylene terephthalate (PET) and polyethylene naphthalene-2,6-dicarboxylate (PEN).

The anode may include a conductive metal, a metal oxide, or a conductive polymer. Holes in the anode can be easily injected into a hole injection layer (HIL), a hole transport layer (HTL), or a light-emitting layer. In one embodiment, an absolute value of a difference between work function of the anode and HOMO energy level or valence band energy level of an emitter of the light-emitting layer, or a p-type semiconductor material of the HIL, the HTL, or an electron blocking layer (EBL) is less than 0.5 eV, preferably less than 0.3 eV, more preferably less than 0.2 eV. Examples of materials of the anode include, but not limited to, Al, Cu, Au, Ag, Mg, Fe, Co, Ni, Mn, Pd, Pt, ITO, zinc oxide doped with aluminum (AZO), and the like. Other suitable materials are known, and ordinary skilled in the art can easily select. Any suitable technology can be used for depositing the materials of the anode, such as a physical vapor deposition method, including RF magnetron sputtering, vacuum thermal evaporation, electron beam (e-beam), or the like. In some embodiments, the anode has a patterned structure. A patterned ITO conductive substrate is commercially available and can be used to prepare the device according to the disclosure.

The cathode may include a conductive metal or a metal oxide. Electrons in the cathode can be easily injected into an electron injection layer (EIL), an electron transport layer (ETL), or the light-emitting layer. In one embodiment, an absolute value of a difference between work function of the cathode and LUMO energy level or valence band energy level of an emitter of the light-emitting layer, or a n-type semiconductor material of the EIL, the ETL, or a hole blocking layer (HBL) is less than 0.5 eV, preferably less than 0.3 eV, more preferably less than 0.2 eV. Theoretically, all materials that can be used for the cathode of OLEDs can be used as materials of the cathode of the devices according to the disclosure. Examples of the materials of the cathode include, but not limited to, Al, Au, Ag, Ca, Ba, Mg, LiF/Al, MgAg alloy, BaF₂/Al, Cu, Fe, Co, Ni, Mn, Pd, Pt, ITO, and the like. Any suitable technology can be used for depositing the materials of the cathode, such as a physical vapor deposition method including RF magnetron sputtering, vacuum thermal evaporation, electron beam (e-beam), or the like.

The OLED may further include other functional layers, such as HIL, HTL, EBL, EIL, ETL, and HBL. The materials suitable for use in these functional layers have been described in detail above and can also refer to patent applications WO 2010/135519 A1, US 2009/0134784 A1, and WO 2011/110277 A1. All the contents of these three patent applications are incorporated herein by reference in their entirety in this context.

The light-emitting device according to the disclosure has an emission wavelength ranging from 300 nm to 1,200 nm, preferably 350 nm to 1000 nm, more preferably 400 nm to 900 nm.

The disclosure further relates to the uses of the electroluminescence device according to the disclosure in various electronic devices, which include, but not limited to, display devices, illumination devices, light sources, sensors, and the like.

The following contents will provide a description of the disclosure in conjunction with preferred examples, but not limited to these. It can be understood that, the claims summarize the scope of the disclosure. Under the guidance of concepts of the disclosure, those skilled in the art should be aware that certain changes made to each example of the disclosure will be covered by the claims.

### EXAMPLES

### 1. Synthesis of Compounds

### Synthesis Example 1: Synthesis of Organic Compound (1)

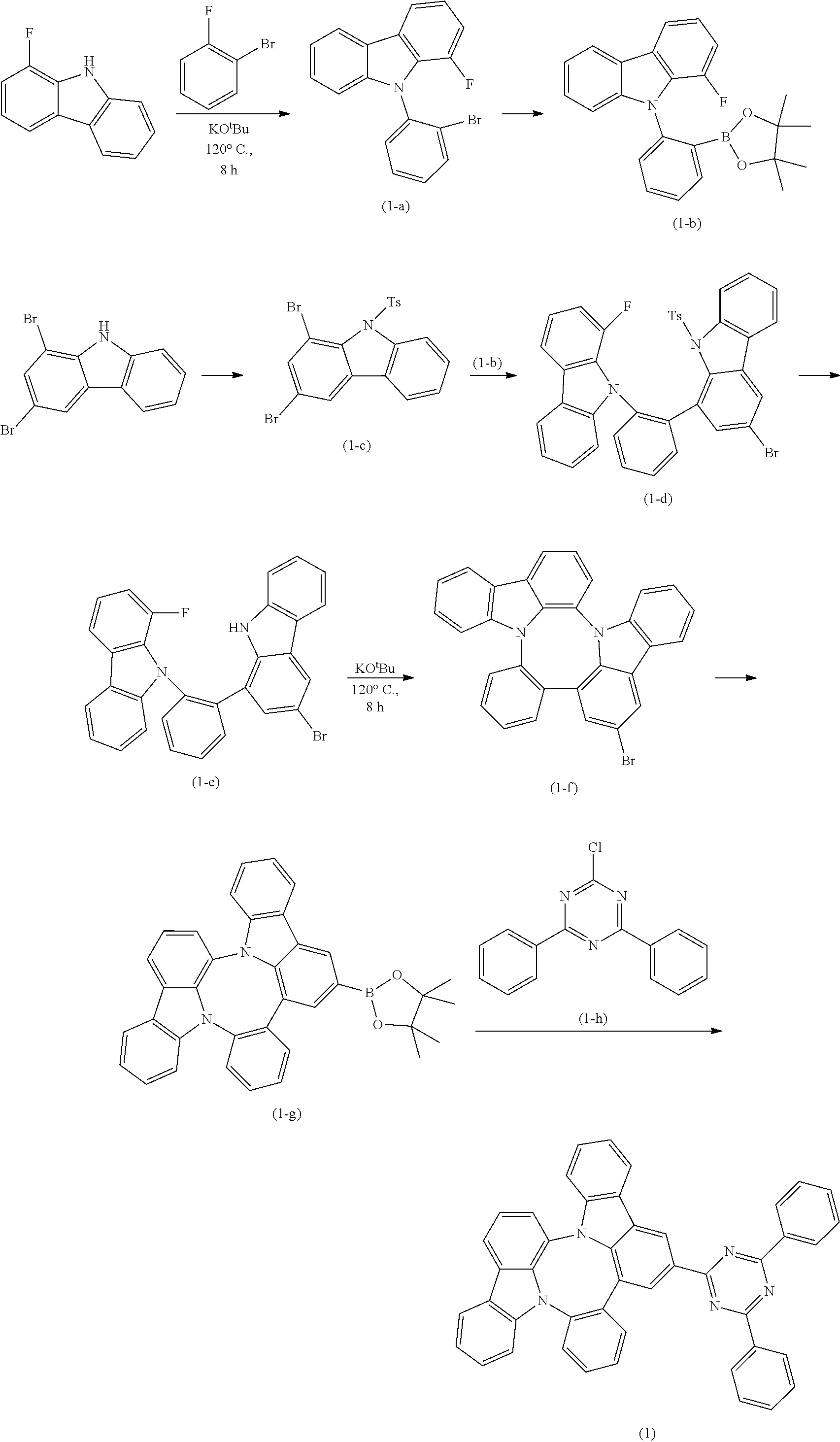

### Synthesis of Intermediate (1-a)

2-bromofluorobenzene (1.5 eq), 1-fluorocarbazole (1 eq), and potassium tert-butanol (5 eq) were placed into a single-necked flask, and 250 mL of DMF was added. The reaction solution was reacted at 120° C. for 8 hours. After the reaction was completed, the reaction solution was dried by rotary evaporation under vacuum, and washed with dichloromethane to obtain solids. Then the solids that cannot be completely dissolved was filtered out to obtain a filtrate. The filtrate was dried by rotary evaporation, and separated and purified by a silica gel chromatography column to obtain a white solid, namely the intermediate (1-a) with yield of 89%.

### Synthesis of Intermediate (1-b)

bis(pinacolato)diboron (1.5 eq), the intermediate (1-a) (1 eq), Pd(dppf)₂Cl₂ (0.05 eq), and potassium acetate (4 eq) were placed into a dry two-necked flask, then 250 mL of a mixture of dioxane and water in a ratio of 3:1 was added. The reaction solution was stirred at 90° C. for 12 hours and cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (1-b) with yield of 86%.

### Synthesis of Intermediate (1-c)

1,3-dibromocarbazole (1 eq) and sodium hydroxide (3 eq) were placed into a dry two-necked flask, 250 mL of a mixture of tetrahydrofuran and water in a ratio of 4:1 was added, and 4-toluenesulfonyl chloride (1.3 eq) was slowly added at 0° C. The reaction solution was stirred and reacted at 0° C. for 12 hours, then raised to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (1-c) with yield of 97%.

### Synthesis of Intermediate (1-d)

The intermediate (1-b) (1 eq), the intermediate (1-c) (1.2 eq), Pd(PPh₃)₄ (0.05 eq), and potassium carbonate (4 eq) were placed into a dry two-necked flask, then 250 mL of a mixture of dioxane and water in a ratio of 3:1 was added. The reaction solution was stirred at 90° C. for 12 hours, then cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (1-d) with yield of 83%.

### Synthesis of Intermediate (1-e)

The intermediate (1-d) (1 eq) was placed into a dry single-necked flask, 250 mL of tetrahydrofuran was added to dissolve it, then concentrated HCl (10 eq) was slowly added at 0° C. After the reaction solution was stirred and reacted for 4 hours, it was dried by rotary evaporation, separated with dichloromethane and water, dried with magnesium sulfate, and dried by rotary evaporation again, then separated and purified by a silica gel chromatography column to obtain a solid intermediate (1-e) with yield of 95%.

### Synthesis of Intermediate (1-f)

The intermediate (1-e) (1 eq) and potassium tert-butanol (5 eq) were placed into a single-necked flask, and 250 mL of DMF was added. The reaction solution was reacted at 120° C. for 8 hours. After the reaction was completed, the reaction solution was dried by rotary evaporation under vacuum, and washed with dichloromethane to obtain solids. Then the solids that cannot be completely dissolved was filtered out to obtain a filtrate. The filtrate was dried by rotary evaporation, and separated and purified by a silica gel chromatography column to obtain a white solid, namely the intermediate (1-f) with yield of 68%.

### Synthesis of Intermediate (1-g)

bis(pinacolato)diboron (1.5 eq), the intermediate (1-f) (1 eq), Pd(dppf)₂Cl₂ (0.05 eq), and potassium acetate (4 eq) were placed into a dry two-necked flask, then 250 mL of a mixture of dioxane and water in a ratio of 3:1 was added. The reaction solution was stirred at 90° C. for 12 hours and cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (1-g) with yield of 70%.

### Synthesis of Organic Compound (1)

The intermediate (1-g) (1 eq), the intermediate (1-h) (1.5 eq), Pd(PPh₃)₄ (0.05 eq), and potassium carbonate (4 eq) were placed into a dry two-necked flask, then 250 mL of a mixture of dioxane and water in a ratio of 3:1 was added. The reaction solution was stirred at 90° C. for 12 hours, then cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid, namely the organic compound (1) with yield of 80%. Mass spectrometry peak: m/z=673.2334 [M]⁺.

### Synthesis Example 2: Synthesis of Organic Compound (2)

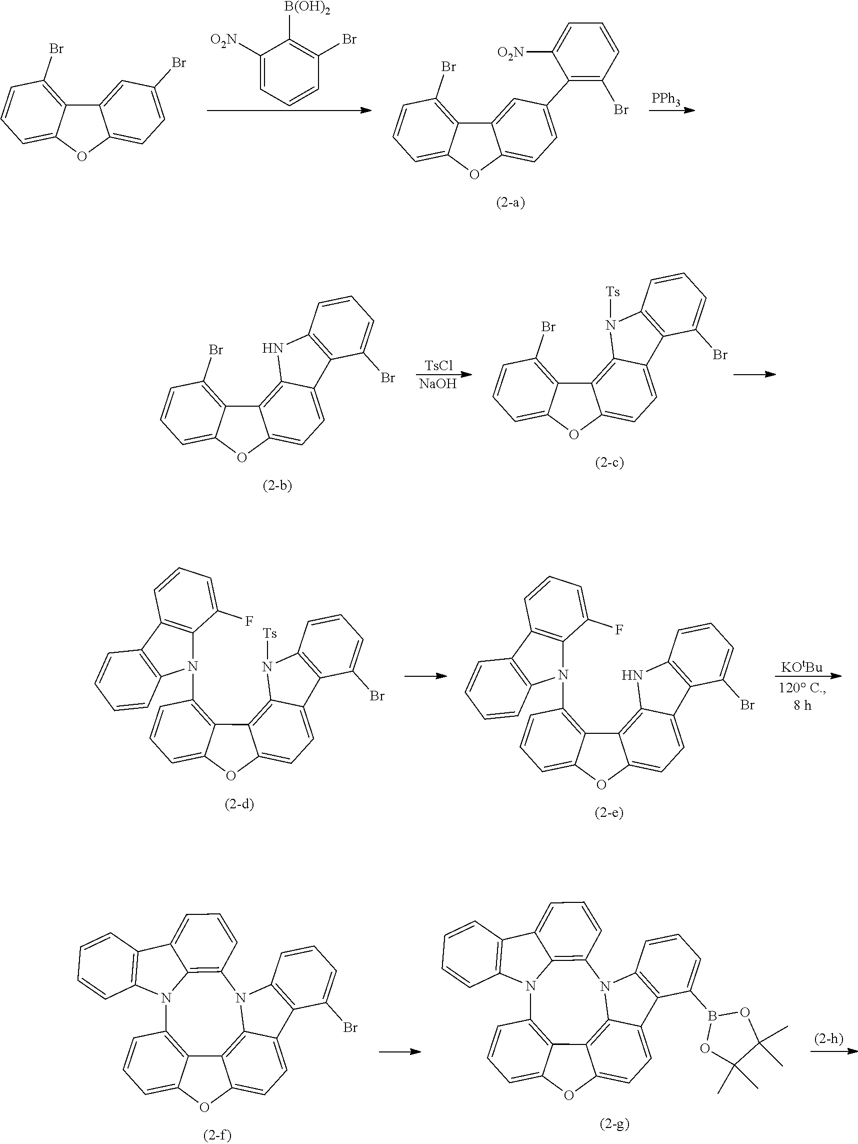

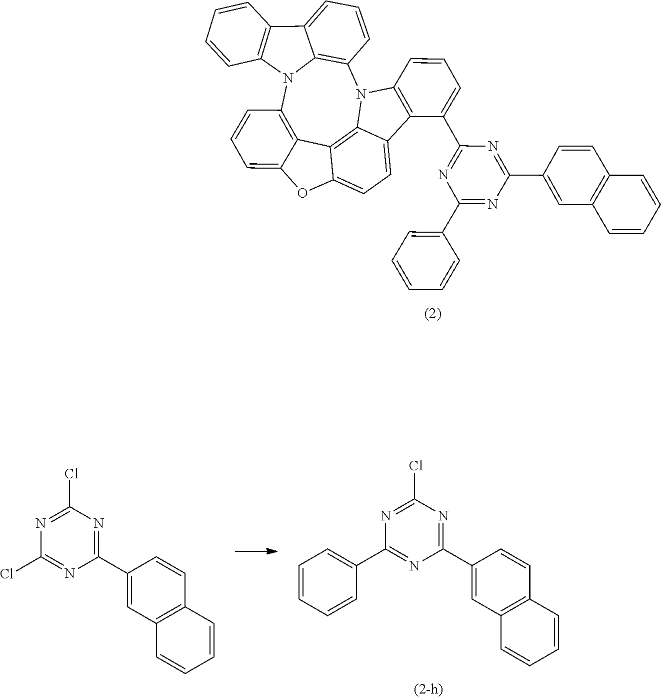 [SMILES: c1ccc(-c2nc(-c3ccc4ccccc4c3)nc(-c3cccc4c3c3ccc5oc6cccc7c6c5c3n4c3cccc4c5ccccc5n7c43)n2)cc1 ; Clc1nc(Cl)nc(-c2ccc3ccccc3c2)n1 ; Clc1nc(-c2ccccc2)nc(-c2ccc3ccccc3c2)n1]

### Synthesis of Intermediate (2-a)

According to the synthesis method of the intermediate (1-d), the differences were in that 1,8-dibromodibenzofuran was substitute for the intermediate (1-b) and 2-bromo-6-nitrophenylboronic acid was substitute for the intermediate (1-c) to obtain a solid intermediate (2-a) with yield of 85%.

### Synthesis of Intermediate (2-b)

The intermediate (2-a) (1 eq) and triphenylphosphine (2 eq) were placed into a dry two-necked flask, then 100 mL of 1,2-dichlorobenzene was added as the solvent. The reaction solution was stirred and reacted at 200° C. for 24 hours, then cooled to room temperature. After the reaction was completed, the solvent was evaporated under vacuum. Then the reaction solution was separated with dichloromethane and water, dried with magnesium sulfate, and dried by rotary evaporation again, then separated and purified by a silica gel chromatography column to obtain a solid intermediate (2-b) with yield of 72%.

### Synthesis of Intermediate (2-c)

According to the synthesis method of the intermediate (1-c), the difference was in that the intermediate (2-b) was substitute for 1,3-dibromocarbazole to obtain a solid intermediate (2-c) with yield of 90%.

### Synthesis of Intermediate (2-d)

The intermediate (2-c) (1 eq), 1-fluorocarbazole (1.2 eq), and potassium carbonate (4 eq) were placed into a dry two-necked flask, 100 mL of nitrobenzene was added as the solvent, and a small amount (about 0.1 g) of copper metal was added as the catalyst. The reaction solution was heated to 200° C. and reacted for 12 hours, then cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (2-d) with yield of 54%.

### Synthesis of Intermediate (2-e)

According to the synthesis method of the intermediate (1-e), the difference was in that the intermediate (2-d) was substitute for the intermediate (1-d) to obtain a solid intermediate (2-e) with yield of 81%.

### Synthesis of Intermediate (2-f)

According to the synthesis method of the intermediate (1-f), the difference was in that the intermediate (2-e) was substitute for the intermediate (1-e), to obtain a white solid intermediate (2-f) with yield of 53%.

### Synthesis of Intermediate (2-g)

According to the synthesis method of the intermediate (1-g), the difference was in that the intermediate (2-f) was substitute for the intermediate (1-f) to obtain a solid intermediate (2-g) with yield of 59%.

### Synthesis of Intermediate (2-h)

According to the synthesis method of the intermediate (1-d), the differences were in that 2,4-dichloro-6-naphthalen-2-yl-[1,3,5]triazine was substitute for the intermediate (1-b) and phenylboronic acid was substitute for the intermediate (1-c) to obtain a solid intermediate (2-h) with yield of 66%.

### Synthesis of Organic Compound (2)

According to the synthesis method of the compound (1), the differences were in that the intermediate (2-g) was substitute for the intermediate (1-g) and the intermediate (2-h) was substitute for the intermediate (1-h) to obtain a solid organic compound (2) with yield of 70%. Mass spectrometry peak: m/z=701.2236 [M]⁺.

### Synthesis Example 3: Synthesis of Organic Compound (3)

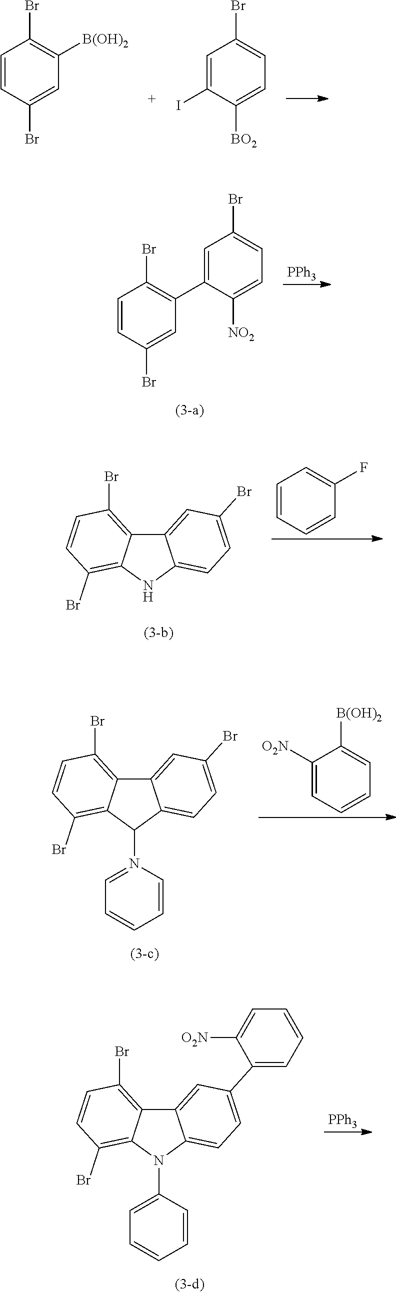

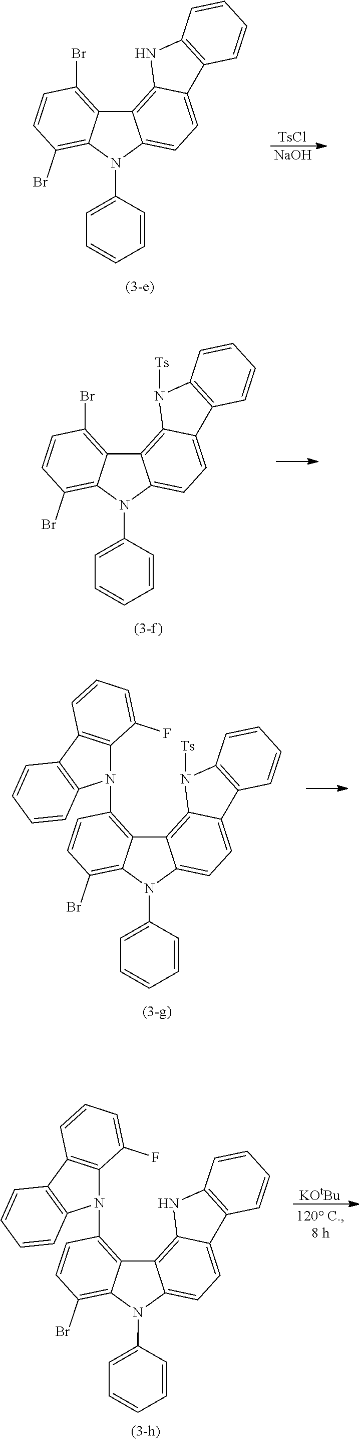

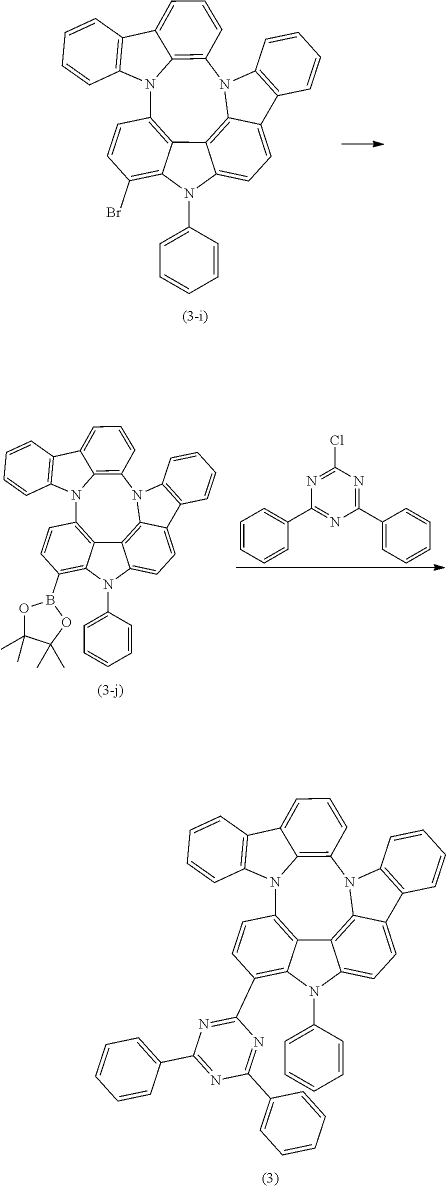

### Synthesis of Intermediate (3-a)

According to the synthesis method of the intermediate (1-d), the differences were in that 2,5-dibromophenylboronic acid was substitute for the intermediate (1-b) and 1-nitro-2-iodo-4-bromobenzene was substitute for the intermediate (1-c) to obtain a solid organic compound (3-a) with yield of 79%.

### Synthesis of Intermediate (3-b)

According to the synthesis method of the intermediate (2-b), the difference was in that the intermediate (3-a) was substitute for the intermediate (2-a) to obtain a solid intermediate (3-b) with yield of 69%.

### Synthesis of Intermediate (3-c)

According to the synthesis method of the intermediate (1-a), the differences were in that the intermediate (3-b) was substitute for 2-bromofluorobenzene and fluorobenzene was substitute for 1-fluorocarbazole to obtained a white solid intermediate (3-c) with yield of 93%.

### Synthesis of Intermediate (3-d)

According to the synthesis method of the intermediate (1-d), the differences were in that the intermediate (3-c) was substitute for the intermediate (1-b) and 2-nitrophenylboronic acid was substitute for the intermediate (1-c) to obtain a solid intermediate (3-d) with yield of 89%.

### Synthesis of Intermediate (3-e)

According to the synthesis method of the intermediate (2-b), the difference was in that the intermediate (3-d) was substitute for the intermediate (2-a) to obtain a solid intermediate (3-e) with yield of 57%.

### Synthesis of Intermediate (3-f)

According to the synthesis method of the intermediate (1-c), the difference was in that the intermediate (3-e) was substitute for 1,3-dibromocarbazole to obtain a solid intermediate (3-f) with yield of 83%.

### Synthesis of Intermediate (3-g)

According to the synthesis method of the intermediate (2-d), the difference was in that the intermediate (3-f) was substitute for the intermediate (2-c) to obtain a solid intermediate (3-g) with yield of 56%.

### Synthesis of Intermediate (3-h)

According to the synthesis method of the intermediate (1-e), the difference was in that the intermediate (3-g) was substitute for the intermediate (1-d) to obtain a solid intermediate (3-h) with yield of 99%.

### Synthesis of Intermediate (3-i)

According to the synthesis method of the intermediate (1-f), the difference was in that the intermediate (3-h) was substitute for the intermediate (1-e), to obtain a white solid intermediate (3-i) with yield of 35%.

### Synthesis of Intermediate (3-j)

According to the synthesis method of the intermediate (1-g), the difference was in that the intermediate (3-i) was substitute for the intermediate (1-f) to obtain a solid intermediate (3-j) with yield of 43%.

### Synthesis of Organic Compound (3)

According to the synthesis method of the compound (1), the difference was in that the intermediate (3-j) was substitute for the intermediate (1-g) to obtain a solid organic compound (3) with yield of 68%. Mass spectrometry peak: m/z=726.2514 [M]⁺.

### Synthesis Example 4: Synthesis of Organic Compound (4)

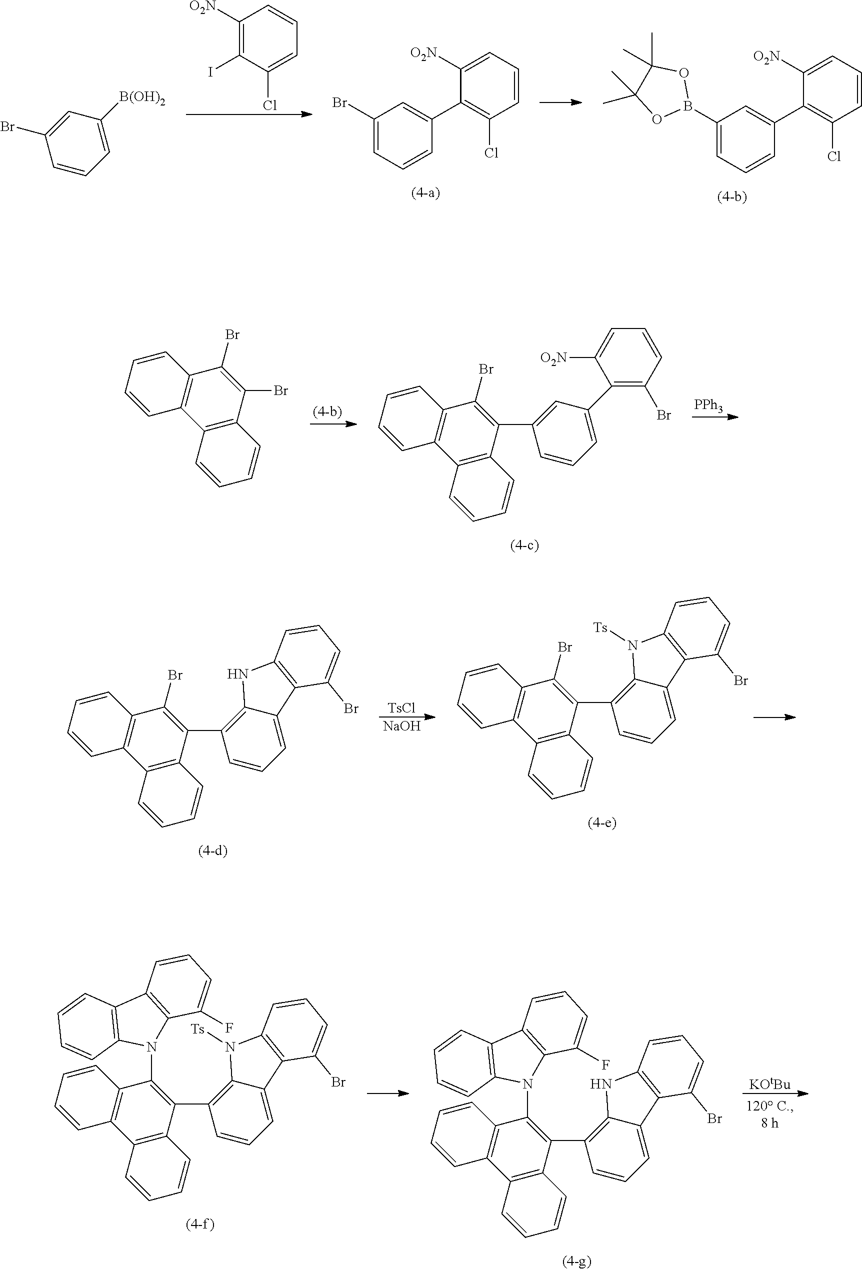

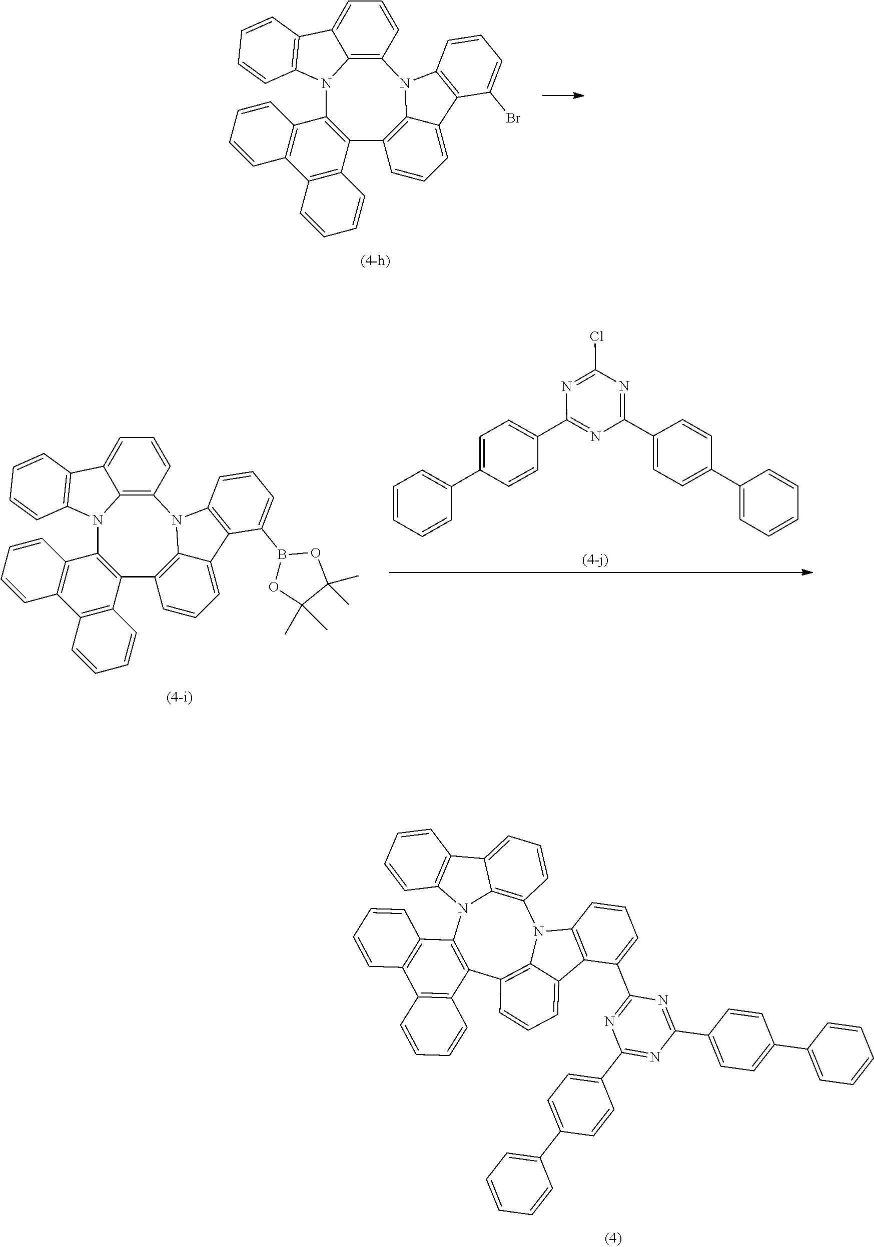 [SMILES: Brc1cccc2c1c1cccc3c4c5ccccc5c5ccccc5c4n4c5ccccc5c5cccc(c54)n2c13 ; CC1(C)OB(c2cccc3c2c2cccc4c5c6ccccc6c6ccccc6c5n5c6ccccc6c6cccc(c65)n3c24)OC1(C)C ; Clc1nc(-c2ccc(-c3ccccc3)cc2)nc(-c2ccc(-c3ccccc3)cc2)n1 ; c1ccc(-c2ccc(-c3nc(-c4ccc(-c5ccccc5)cc4)nc(-c4cccc5c4c4cccc6c7c8ccccc8c8ccccc8c7n7c8ccccc8c8cccc(c87)n5c46)n3)cc2)cc1]

### Synthesis of Intermediate (4-a)

According to the synthesis method of the intermediate (1-d), the differences were in that 1-chloro-2-iodo-3-nitrobenzene was substitute for the intermediate (1-b) and 3-bromophenylboronic acid was substitute for the intermediate (1-c) to obtain a solid intermediate (4-a) with yield of 53%.

### Synthesis of Intermediate (4-b)

According to the synthesis method of the intermediate (1-b), the difference was in that the intermediate (4-a) was substitute for the intermediate (1-a) to obtain a solid intermediate (4-b) with yield of 68%.

### Synthesis of Intermediate (4-c)

According to the synthesis method of the intermediate (1-d), the differences were in that 9,10-dibromophenone was substitute for the intermediate (1-b) and the intermediate (4-b) was substitute for the intermediate (1-c) to obtain a solid intermediate (4-c) with yield of 56%.

### Synthesis of Intermediate (4-d)

According to the synthesis method of the intermediate (2-b), the difference was in that the intermediate (4-c) was substitute for the intermediate (2-a) to obtain a solid intermediate (4-d) with yield of 70%.

### Synthesis of Intermediate (4-e)

According to the synthesis method of the intermediate (1-c), the difference was in that the intermediate (4-d) was substitute for 1,3-dibromocarbazole to obtain a solid intermediate (4-e) with yield of 80%.

### Synthesis of Intermediate (4-f)

According to the synthesis method of the intermediate (2-d), the difference was in that the intermediate (4-e) was substitute for the intermediate (2-c) to obtain a solid intermediate (4-f) with yield of 56%.

### Synthesis of Intermediate (4-g)

According to the synthesis method of the intermediate (1-e), the difference was in that the intermediate (4-f) was substitute for the intermediate (1-d) to obtain a solid intermediate (4-g) with yield of 90%.

### Synthesis of Intermediate (4-h)

According to the synthesis method of the intermediate (1-f), the difference was in that the intermediate (4-g) was substitute for the intermediate (1-e), to obtained a white solid intermediate (4-h) with yield of 44%.

### Synthesis of Intermediate (4-i)

According to the synthesis method of the intermediate (1-g), the difference was in that the intermediate (4-h) was substitute for the intermediate (1-f) to obtain a solid intermediate (4-i) with yield of 61%.

### Synthetic organic compounds (4)

According to the synthesis method of the compound (1), the differences were in that the intermediate (4-i) was substitute for the intermediate (1-g) and the intermediate (4-j) was substitute for the intermediate (1-h) to obtain a solid organic compound (4) with yield of 72%. Mass spectrometry peak: m/z=889.3217 [M]⁺.

### Synthesis Example 5: Synthesis of Organic Compound (5)

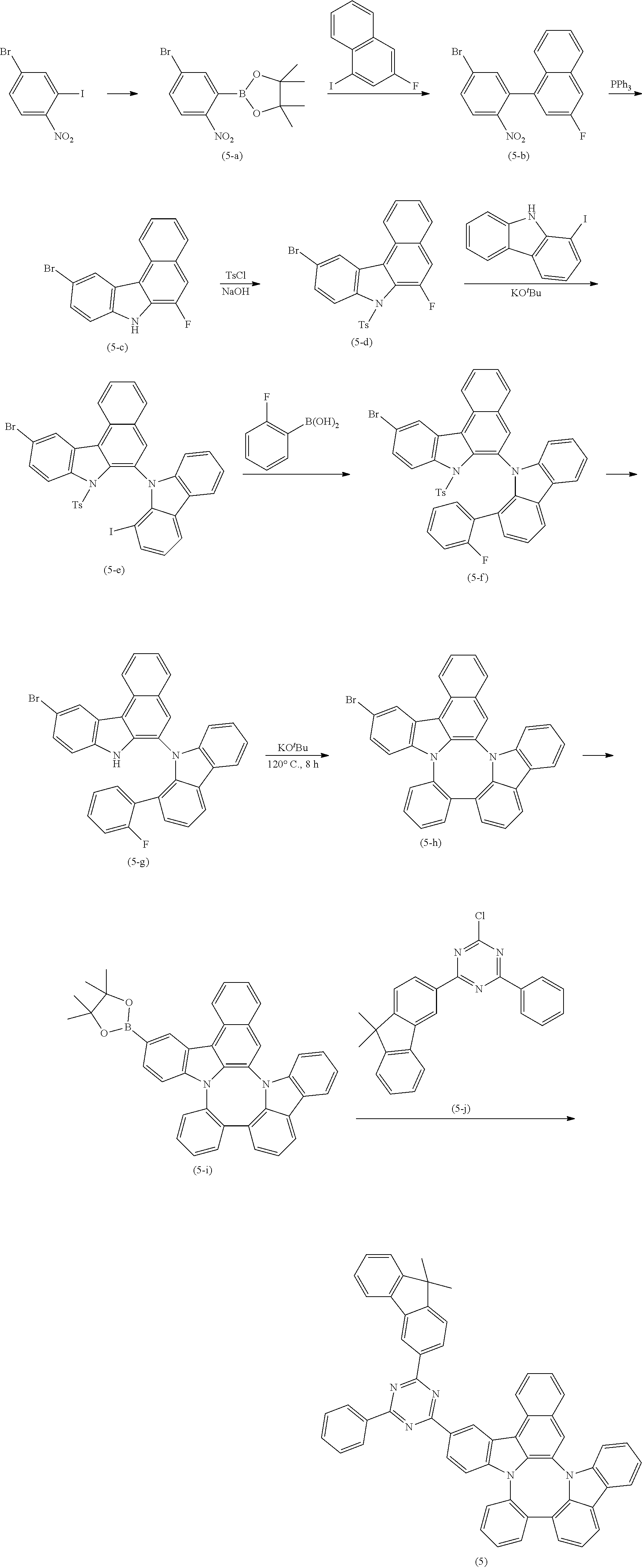

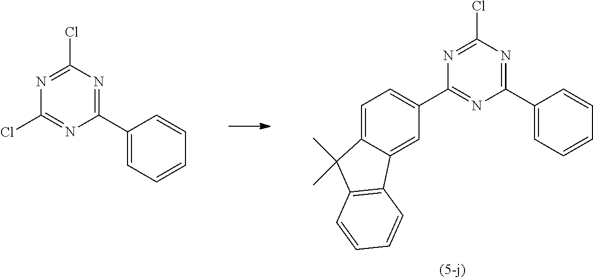 [SMILES: Clc1nc(Cl)nc(-c2ccccc2)n1 ; CC1(C)c2ccccc2-c2cc(-c3nc(Cl)nc(-c4ccccc4)n3)ccc21]

### Synthesis of Intermediate (5-a)

bis(pinacolato)diboron (1.5 eq), 1-nitro-2-iodo-4-bromobenzene (1-a) (1 eq), Pd(dppf)₂Cl₂ (0.05 eq), and potassium acetate (4 eq) were placed into a dry two-necked flask, and 250 mL of a mixture of dioxane and water in a ratio of 3:1 was added. The reaction solution was stirred at 90° C. for 12 hours, then cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (5-a) with yield of 66%.

### Synthesis of Intermediate (5-b)

According to the synthesis method of the intermediate (1-d), the differences were in that 1-iodo-3-fluoronaphthalene was substitute for the intermediate (1-b) and the intermediate (5-a) was substitute for the intermediate (1-c) to obtain a solid intermediate (5-b) with yield of 51%.

### Synthesis of Intermediate (5-c)

According to the synthesis method of the intermediate (2-b), the difference was in that the intermediate (5-b) was substitute for the intermediate (2-a) to obtain a solid intermediate (5-c) with yield of 50%.

### Synthesis of Intermediate (5-d)

According to the synthesis method of the intermediate (1-c), the difference was in that the intermediate (5-c) was substitute for 1,3-dibromocarbazole to obtain a solid intermediate (5-d) with yield of 90%.

### Synthesis of Intermediate (5-e)

According to the synthesis method of the intermediate (1-a), the differences were in that the intermediate (5-d) was substitute for 2-bromofluorobenzene and 1-iodocarazole was substitute for 1-fluorocarbazole to obtain a white solid intermediate (5-e) with yield of 80%.

### Synthesis of Intermediate (5-f)

According to the synthesis method of the intermediate (1-d), the differences were in that the intermediate (5-e) was substitute for the intermediate (1-b) and ortho-fluoroboric acid was substitute for the intermediate (1-c) to obtain a solid intermediate (5-f) with yield of 42%.

### Synthesis of Intermediate (5-g)

According to the synthesis method of the intermediate (1-e), the difference was in that the intermediate (5-f) was substitute for the intermediate (1-d) to obtain a solid intermediate (5-g) with yield of 87%.

### Synthesis of Intermediate (5-h)

According to the synthesis method of the intermediate (1-f), the difference was in that the intermediate (5-g) was substitute for the intermediate (1-e) to obtain a white solid intermediate (5-h) with yield of 35%.

### Synthesis of Intermediate (5-i)

According to the synthesis method of the intermediate (1-g), the difference was in that the intermediate (5-h) was substitute for the intermediate (1-f) to obtain a solid intermediate (5-i) with yield of 70%.

### Synthesis of Intermediate (5-j)

According to the synthesis method of the intermediate (1-d), the differences were in that 2,4-dichloro-6-phenyl-1,3,5-triazine was substitute for the intermediate (1-b) and 9,9-dimethylfluoren-2-boronic acid was substitute for the intermediate (1-c) to obtain a solid intermediate (5-j) with yield of 54%.

### Synthesis of Organic Compound (5)

According to the synthesis method of the compound (1), the differences were in that the intermediate (5-i) was substitute for the intermediate (1-g) and the intermediate (5-j) was substitute for the intermediate (1-h) to obtain a solid organic compound (5) with yield of 55%. Mass spectrometry peak: m/z=803.3041 [M]⁺.

### Synthesis Example 6: Synthesis of Organic Compound (6)

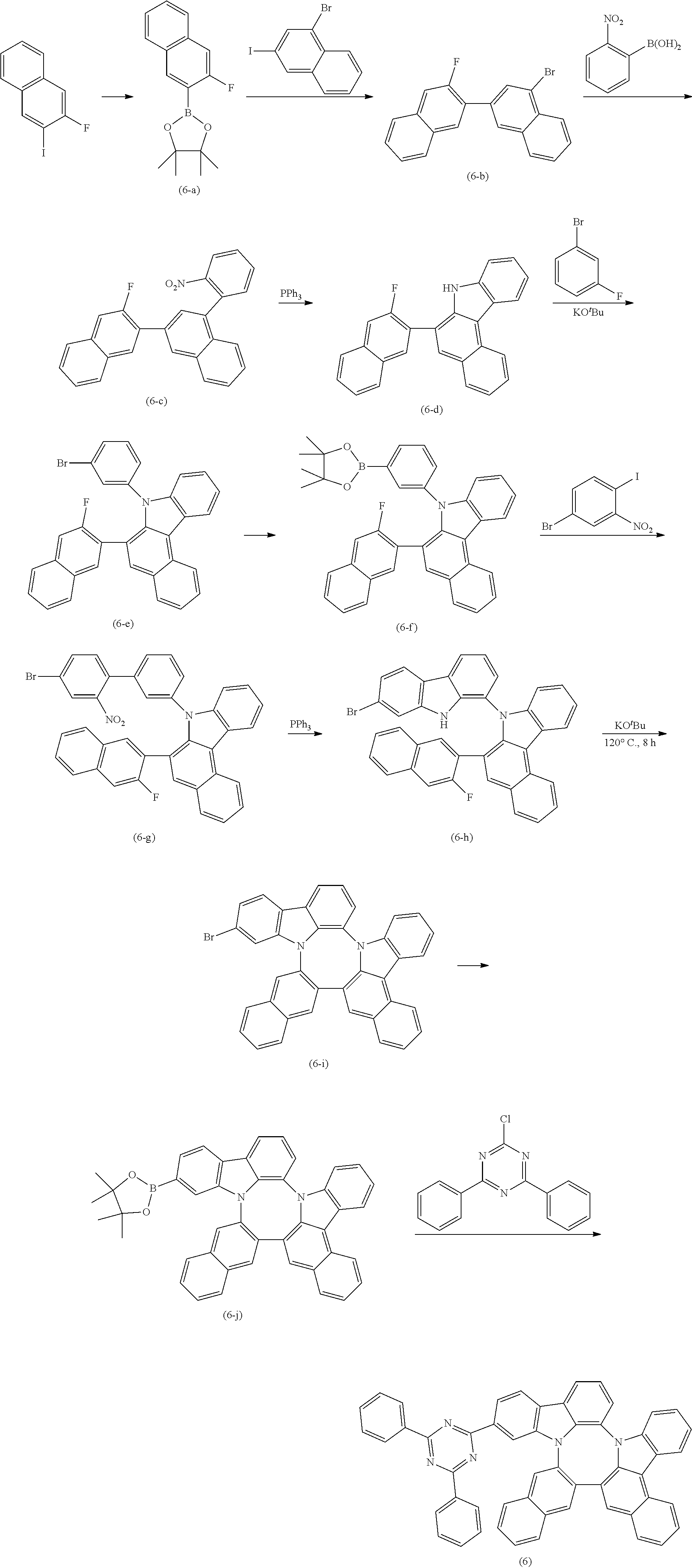

### Synthesis of Intermediate (6-a)

bis(pinacolato)diboron (1.5 eq), 2-fluoro-6-iodonaphthalene (1 eq), Pd(dppf)₂Cl₂ (0.05 eq), and potassium acetate (4 eq) were placed into a dry two-necked flask, and 250 mL of a mixture of dioxane and water in a ratio of 3:1 was added. The reaction solution was stirred at 90° C. for 12 hours, then cooled to room temperature. After the reaction was completed, the reaction solution was dried by rotary evaporation, separated with dichloromethane and water, dried with magnesium sulfate, and dried by rotary evaporation again, then separated and purified by a silica gel chromatography column to obtain a solid intermediate (6-a) with yield of 89%.

### Synthesis of Intermediate (6-b)

According to the synthesis method of the intermediate (1-d), the differences were in that 1-bromo-3-iodo-naphthalene was substitute for intermediate (1-b) and the intermediate (6-a) was substitute for the intermediate (1-c) to obtain a solid intermediate (6-b) with yield of 78%.

### Synthesis of Intermediate (6-c)

According to the synthesis method of the intermediate (1-d), the difference was in that 2-nitrophenylboronic acid was substitute for the intermediate (1-b) and the intermediate (6-b) was substitute for the intermediate (1-c) to obtain a solid intermediate (6-c) with yield of 70%.

### Synthesis of Intermediate (6-d)

According to the synthesis method of the intermediate (2-b), the difference was in that the intermediate (6-c) was substitute for the intermediate (2-a) to obtain a solid intermediate (6-d) with yield of 43%.

### Synthesis of Intermediate (6-e)

According to the synthesis method of the intermediate (1-a), the difference was in that the intermediate (6-d) was substitute for 2-bromofluorobenzene and 3-bromofluorobenzene was substitute for 1-fluorocarbazole to obtain a white solid intermediate (6-e) with yield of 66%.

### Synthesis of Intermediate (6-f)

According to the synthesis method of the intermediate (1-b), the difference was in that the intermediate (6-e) was substitute for the intermediate (1-a) to obtain a solid intermediate (6-f) with yield of 80%.

### Synthesis of Intermediate (6-g)

According to the synthesis method of the intermediate (1-d), the differences were in that 1-nitro-2-iodo-4-bromobenzene was substitute for the intermediate (1-b) and the intermediate (6-f) was substitute for the intermediate (1-c) to obtain a solid intermediate (6-g) with yield of 78%.

### Synthesis of Intermediate (6-h)

According to the synthesis method of the intermediate (2-b), the difference was in that the intermediate (6-g) was substitute for the intermediate (2-a) to obtain a solid intermediate (6-h) with yield of 53%.

### Synthesis of Intermediate (6-i)

According to the synthesis method of the intermediate (1-f), the difference was in that the intermediate (6-h) was substitute for the intermediate (1-e) to obtain a white solid intermediate (6-i) with yield of 31%.

### Synthesis of Intermediate (6-j)

According to the synthesis method of the intermediate (1-g), the difference was in that the intermediate (6-i) was substitute for the intermediate (1-f) to obtain a solid intermediate (6-j) with yield of 90%.

### Synthesis of Organic Compound (6)

According to the synthesis method of the compound (1), the difference was in that the intermediate (6-j) was substitute for the intermediate (1-g) to obtain a solid organic compound (6) with yield of 54%. Mass spectrometry peak: m/z=737.2618 [M]⁺.

### Synthesis Example 7

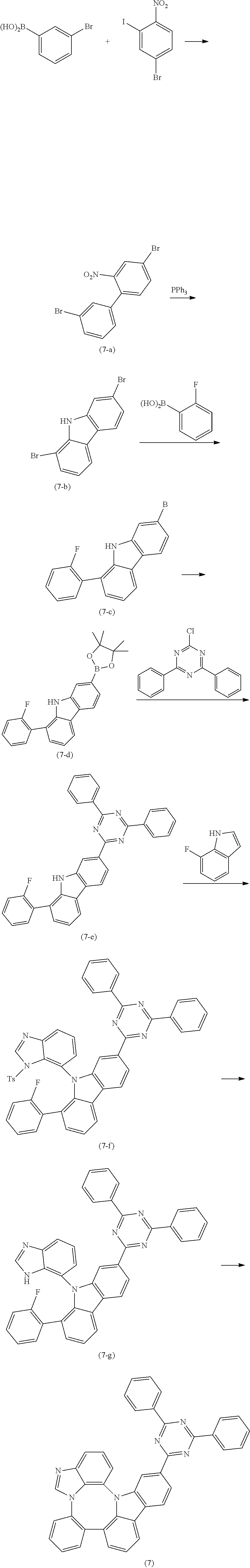

### Synthesis of Intermediate (7-a)

According to the synthesis method of the intermediate (1-d), the differences were in that 3-bromophenylboronic acid was substitute for the intermediate (1-b) and 1-nitro-2-iodo-4-bromobenzene was substitute for the intermediate (1-c) to obtain a solid intermediate (7-a) with yield of 60%.

### Synthesis of Intermediate (7-b)

According to the synthesis method of the intermediate (2-b), the difference was in that the intermediate (7-a) was substitute for the intermediate (2-a) to obtain a solid intermediate (7-b) with yield of 57%.

### Synthesis of Intermediate (7-c)

According to the synthesis method of the intermediate (1-d), the differences were in that 2-fluorophenylboronic acid was substitute for the intermediate (1-b) and the intermediate (7-b) was substitute for the intermediate (1-c) to obtain a solid intermediate (7-c) with yield of 54%.

### Synthesis of Intermediate (7-d)

According to the synthesis method of the intermediate (1-b), the difference was in that the intermediate (7-c) was substitute for the intermediate (1-a) to obtain a solid intermediate (7-d) with yield of 88%.

### Synthesis of Intermediate (7-e)

According to the synthesis method of the intermediate (1-d), the differences were in that the intermediate (7-d) was substitute for the intermediate (1-b) and 2-chloro-4,6-diphenyl-1,3,5-triazine was substitute for the intermediate (1-c) to obtain a solid intermediate (7-e) with yield of 70%.

### Synthesis of Intermediate (7-f)

According to the synthesis method of the intermediate (1-a), the differences were in that the intermediate (7-e) was substitute for 2-bromofluorobenzene and 7-fluorobenzimidazole was substitute for 1-fluorocarbazole to obtain a white solid intermediate (7-f) with yield of 66%.

### Synthesis of Intermediate (7-g)

According to the synthesis method of the intermediate (1-e), the difference was in that the intermediate (7-f) was substitute for the intermediate (1-d) to obtain a solid intermediate (7-g) with yield of 93%.

### Synthesis of Organic Compound (7)

According to the synthesis method of the intermediate (1-f), the difference was in that the intermediate (7-g) was substitute for the intermediate (1-e) to obtain a white solid organic compound (7) with yield of 27%. Mass spectrometry peak: m/z=588.2125 [M]⁺.

### Synthesis Example 8: Synthesis of Organic Compound (8)

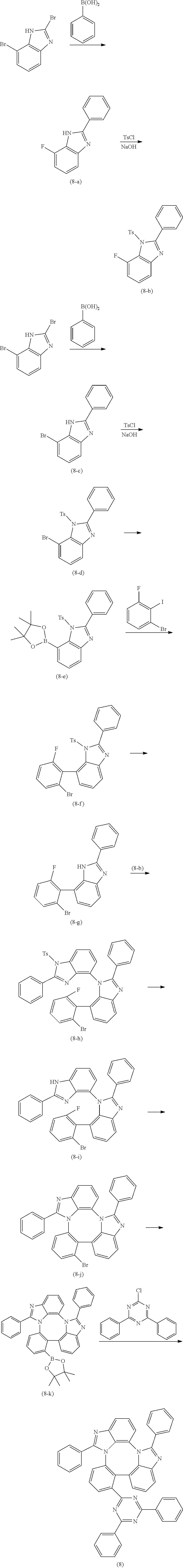

### Synthesis of Intermediate (8-a)

According to the synthesis method of the intermediate (1-d), the differences were in that 2-bromo-7-fluorobenzimidazole was substitute for the intermediate (1-b) and phenylboronic acid was substitute for the intermediate (1-c) to obtain a solid intermediate (8-a) with yield of 90%.

### Synthesis of Intermediate (8-b)

According to the synthesis method of the intermediate (1-c), the difference was in that the intermediate (8-a) was substitute for 1,3-dibromocarbazole to obtain a solid intermediate (8-b) with yield of 95%.

### Synthesis of Intermediate (8-c)

According to the synthesis method of the intermediate (1-d), the differences were in that 2,7-dibromobenzimidazole was substitute for the intermediate (1-b) and phenylboronic acid was substitute for the intermediate (1-c) to obtain a solid intermediate (8-c) with yield of 85%.

### Synthesis of Intermediate (8-d)

According to the synthesis method of the intermediate (1-c), the difference was in that the intermediate (8-c) was substitute for 1,3-dibromocarbazole to obtain a solid intermediate (8-d) with yield of 90%.

### Synthesis of Intermediate (8-e)

According to the synthesis method of the intermediate (1-b), the difference was in that the intermediate (8-d) was substitute for the intermediate (1-a) to obtain a solid intermediate (8-e) with yield of 72%.

### Synthesis of Intermediate (8-f)

According to the synthesis method of the intermediate (1-d), the differences were in that 1-bromo-2-iodo-3-fluorobenzene was substitute for the intermediate (1-b) and the intermediate (8-e) was substitute for the intermediate (1-c) to obtain a solid intermediate (8-f) with yield of 25%.

### Synthesis of Intermediate (8-g)

According to the synthesis method of the intermediate (1-e), the difference was in that the intermediate (8-f) was substitute for the intermediate (1-d) to obtain a solid intermediate (8-g) with yield of 89%.

### Synthesis of Intermediate (8-h)

According to the synthesis method of the intermediate (1-a), the differences were in that the intermediate (8-g) was substitute for 2-bromofluorobenzene and the intermediate (8-b) was substitute for 1-fluorocarbazole to obtain a white solid intermediate (8-h) with yield of 38%.

### Synthesis of Intermediate (8-i)

According to the synthesis method of the intermediate (1-e), the difference was in that the intermediate (8-h) was substitute for the intermediate (1-d) to obtain a solid intermediate (8-i) with yield of 84%.

### Synthesis of Intermediate (8-j)

According to the synthesis method of the intermediate (1-f), the difference was in that the intermediate (8-i) was substitute for the intermediate (1-e) to obtain a white solid intermediate (8-j) with yield of 26%.

### Synthesis of Intermediate (8-k)

According to the synthesis method of the intermediate (1-g), the difference was in that the intermediate (8-j) was substitute for the intermediate (1-f) to obtain a solid intermediate (8-k) with yield of 55%.

### Synthesis of Organic Compound (8)

According to the synthesis method of the compound (1), the difference was in that the intermediate (8-k) was substitute for the intermediate (1-g) to obtain a solid organic compound (8) with yield of 47%. Mass spectrometry peak: m/z=691.2509 [M]⁺.

### Synthesis Example 9: Synthesis of Organic Compound (9)

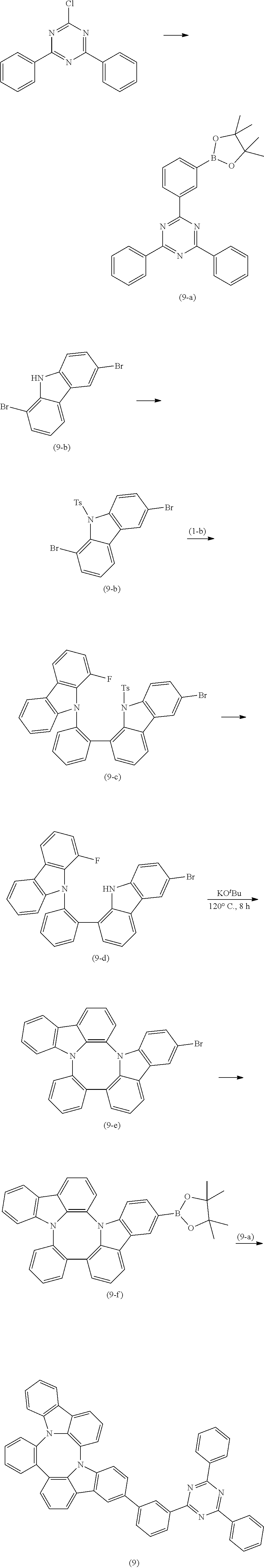

### Synthesis of Intermediate (9-a)

According to the synthesis method of the intermediate (1-d), the differences were in that 2-chloro-4,6-diphenyl-1,3,5-triazine was substitute for the intermediate (1-b) and 1,3-phenyldiboronic acid, bis(pinacol) ester was substitute for the intermediate (1-c) to obtain a solid intermediate (9-a) with yield of 74%.

### Synthesis of Intermediate (9-b)

According to the synthesis method of the intermediate (1-c), the difference was in that 1,6-dibromocarbazole was substitute for 1,3-dibromocarbazole to obtain a solid intermediate (9-b) with yield of 61%.

### Synthesis of Intermediate (9-c)

According to the synthesis method of the intermediate (1-d), the differences were in that the intermediate (1-b) was substitute for the intermediate (1-b) and the intermediate (9-b) was substitute for the intermediate (1-c) to obtain a solid intermediate (9-c) with yield of 67%.

### Synthesis of Intermediate (9-d)

According to the synthesis method of the intermediate (1-e), the difference was in that the intermediate (9-c) was substitute for the intermediate (1-d) to obtain a solid intermediate (9-d) with yield of 94%.

### Synthesis of Intermediate (9-e)

According to the synthesis method of the intermediate (1-f), the difference was in that the intermediate (9-d) was substitute for the intermediate (1-e) to obtain a white solid intermediate (9-e) with yield of 54%.

### Synthesis of Intermediate (9-f)

According to the synthesis method of the intermediate (1-g), the difference was in that the intermediate (9-e) was substitute for the intermediate (1-f) to obtain a solid intermediate (9-f) with yield of 69%.

### Synthesis of Organic Compound (9)

According to the synthesis method of the compound (1), the differences were in that the intermediate (9-f) was substitute for the intermediate (1-g) and the intermediate (9-a) was substitute for the intermediate (1-h) to obtain a solid organic compound (9) with yield of 72%. Mass spectrometry peak: m/z=713.2630 [M]⁺.

### Synthesis Example 10: Synthesis of Organic Compound (10)

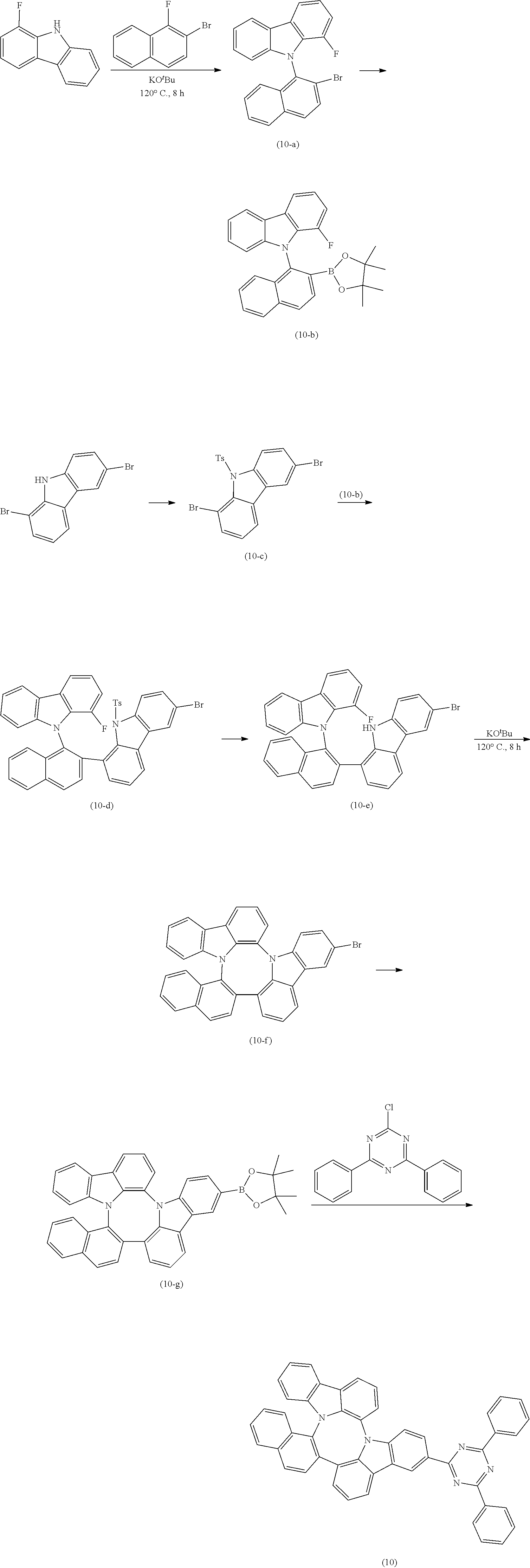

### Synthesis of Intermediate (10-a)

According to the synthesis method of the intermediate (1-a), the difference was in that 2-bromo-1-fluoronaphthalene was substitute for 2-bromofluorobenzene to obtain a white solid intermediate (10-a) with yield of 80%.

### Synthesis of Intermediate (10-b)

According to the synthesis method of the intermediate (1-b), the difference was in that the intermediate (10-a) was substitute for the intermediate (1-a) to obtain a solid intermediate (10-b) with yield of 46%.

### Synthesis of Intermediate (10-c)

According to the synthesis method of the intermediate (1-c), the difference was in that 1,6-dibromocarbazole was substitute for 1,3-dibromocarbazole to obtain a solid intermediate (10-c) with yield of 89%.

### Synthesis of Intermediate (10-d)

According to the synthesis method of the intermediate (1-d), the differences were in that the intermediate (10-b) was substitute for the intermediate (1-b) and the intermediate (10-c) was substitute for the intermediate (1-c) to obtain a solid intermediate (10-d) with yield of 70%.

### Synthesis of Intermediate (10-e)

According to the synthesis method of the intermediate (1-e), the difference was in that the intermediate (10-d) was substitute for the intermediate (1-d) to obtain a solid intermediate (10-e) with yield of 90%.

### Synthesis of Intermediate (10-f)

According to the synthesis method of the intermediate (1-f), the difference was in that the intermediate (10-e) was substitute for the intermediate (1-e) to obtain a white solid intermediate (10-f) with yield of 56%.

### Synthesis of Intermediate (10-g)

According to the synthesis method of the intermediate (1-g), the difference was in that the intermediate (10-f) was substitute for the intermediate (1-f) to obtain a solid intermediate (10-g) with yield of 67%.

### Synthesis of Organic Compound (10)

According to the synthesis method of the compound (1), the difference was in that the intermediate (10-g) was substitute for the intermediate (1-g) to obtain a solid organic compound (10) with yield of 62%. Mass spectrometry peak: m/z=687.2419 [M]⁺.

### Synthesis Example 11: Synthesis of Organic Compound (11)

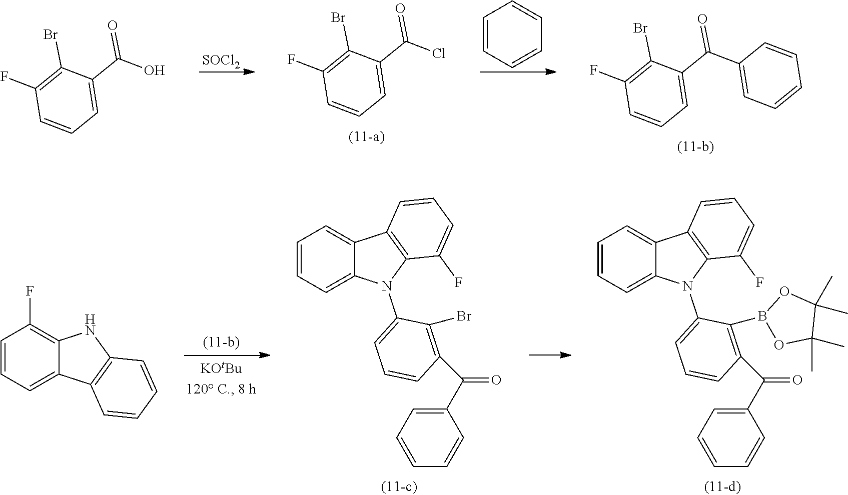

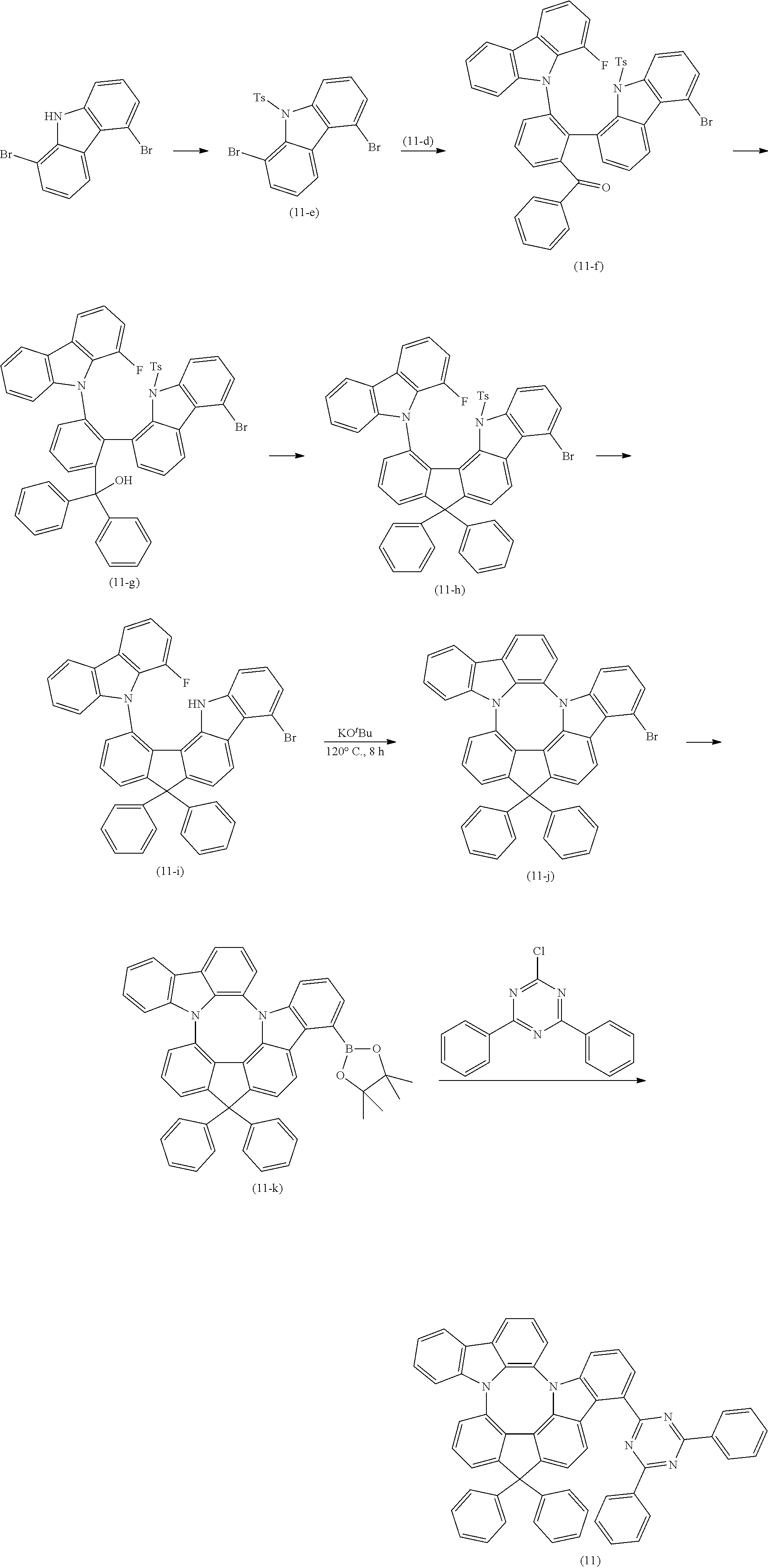

### Synthesis of Intermediate (11-a)

2-bromo-3-fluorobenzoic acid (1 eq) was placed into a single-necked flask, and sulfoxide chloride (10 eq) was added. The reaction solution was reacted at room temperature for 30 minutes. After the reaction was completed, the reaction solution was dried by rotary evaporation under vacuum without purification to obtain a white solid intermediate (11-a) with yield of 93%.

### Synthesis of Intermediate (11-b)

The intermediate (11-a) (1 eq) and aluminum trichloride (1.5 eq) were placed into a single-necked flask, and benzene (100 mL) was added as the reactant and solvent. After the reaction was completed, the reaction solution was dried by rotary evaporation under vacuum, washed with dichloromethane and dried again, then separated and purified by a silica gel chromatography column to obtain a white solid intermediate (11-b) with yield of 71%.

### Synthesis of Intermediate (11-c)

According to the synthesis method of the intermediate (1-a), the difference was in that the intermediate (11-b) was substitute for 2-bromofluorobenzene to obtain a white solid intermediate (11-c) with yield of 65%.

### Synthesis of Intermediate (11-d)

According to the synthesis method of the intermediate (1-b), the difference was in that the intermediate (11-c) was substitute for the intermediate (1-a) to obtain a solid intermediate (11-d) with yield of 54%.

### Synthesis of Intermediate (11-e)

According to the synthesis method of the intermediate (1-c), the difference was in that 1,5-dibromocarbazole was substitute for 1,3-dibromocarbazole to obtain a solid intermediate (11-e) with yield of 85%.

### Synthesis of Intermediate (11-f)

According to the synthesis method of the intermediate (1-d), the differences were in that the intermediate (11-e) was substitute for the intermediate (1-b) and the intermediate (11-d) was substitute for the intermediate (1-c) to obtain a solid intermediate (11-f) with yield of 64%.

### Synthesis of Intermediate (11-g)

The intermediate (11-f) (1 eq) was placed into a dry two-necked flask, in 500 mL of anhydrous tetrahydrofuran was added to dissolve it, then phenylmagnesium bromide (2.1 eq) was added. The reaction solution was stirred at 50° C. for 12 hours. After the reaction was completed, the reaction solution was cooled to room temperature, dried by rotary evaporation, separated with dichloromethane and water, dried with magnesium sulfate, and dried by rotary evaporation again, then separated and purified by a silica gel chromatography column to obtain a solid intermediate (11-g) with yield of 52%.

### Synthesis of Intermediate (11-h)

A dry flask with 250 mL was repeatedly performed on vacuum-pumping and filled three times with nitrogen, then the intermediate (11-g) (1 eq) was placed into the flask, ice acetic acid (50 mL) was added as the solvent, and concentrated sulfuric acid (10 eq) was slowly added. Then the reaction solution was heated to 80° C. and reacted for 2 hours. After the reaction was completed, the reaction solution was poured into 1 L of ice water to precipitate solid. The precipitated solid was filtered, and washed three times with water and methanol to obtain a white solid intermediate (11-h) with yield of 58%.

### Synthesis of Intermediate (11-i)

According to the synthesis method of the intermediate (1-e), the difference was in that the intermediate (11-h) was substitute for the intermediate (1-d) to obtain a solid intermediate (11-i) with yield of 90%.

### Synthesis of Intermediate (11-j)

According to the synthesis method of the intermediate (1-f), the difference was in that the intermediate (11-i) was substitute for the intermediate (1-e) to obtain a white solid intermediate (11-j) with yield of 63%.

### Synthesis of Intermediate (11-k)

According to the synthesis method of the intermediate (1-g), the difference was in that the intermediate (11-i) was substitute for the intermediate (1-f) to obtain a solid intermediate (11-k) with yield of 78%.

### Synthesis of Organic Compound (11)

According to the synthesis method of the compound (1), the difference was in that the intermediate (11-k) was substitute for the intermediate (1-g) to obtain a solid organic compound (11) with yield of 44%. Mass spectrometry peak: m/z=801.2909 [M]⁺.

### Synthesis Example 12: Synthesis of Organic Compound (12)

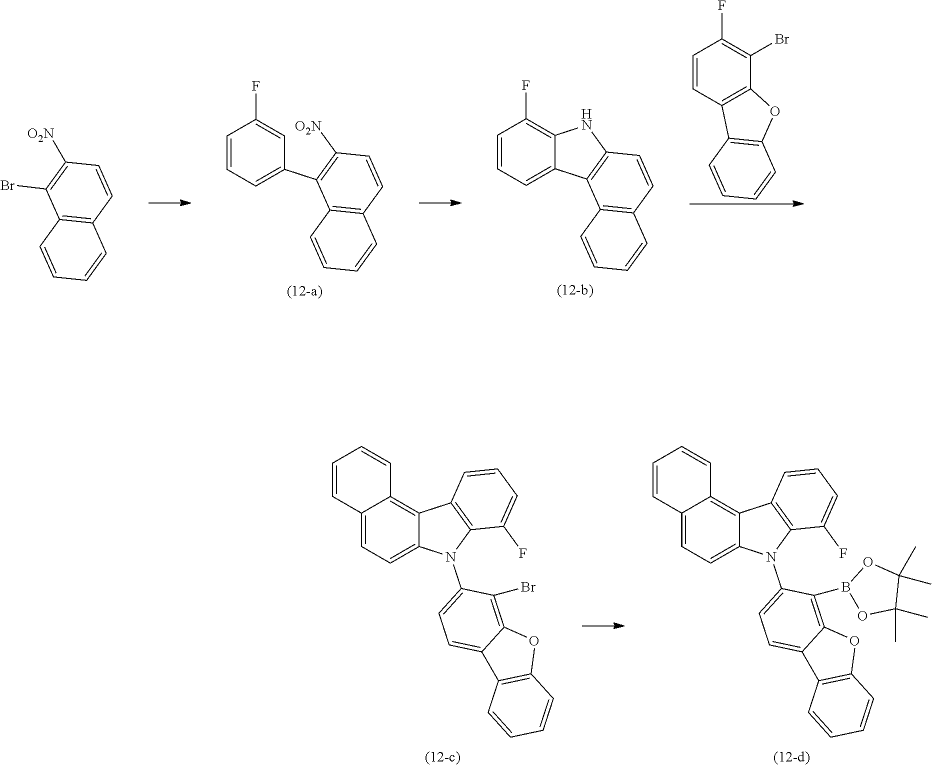 [SMILES: O=[N+]([O-])c1ccc2ccccc2c1Br ; O=[N+]([O-])c1ccc2ccccc2c1-c1cccc(F)c1 ; Fc1cccc2c1[nH]c1ccc3ccccc3c12 ; Fc1ccc2c(oc3ccccc32)c1Br ; Fc1cccc2c3c4ccccc4ccc3n(-c3ccc4c(oc5ccccc54)c3Br)c12 ; CC1(C)OB(c2c(-n3c4ccc5ccccc5c4c4cccc(F)c43)ccc3c2oc2ccccc23)OC1(C)C]

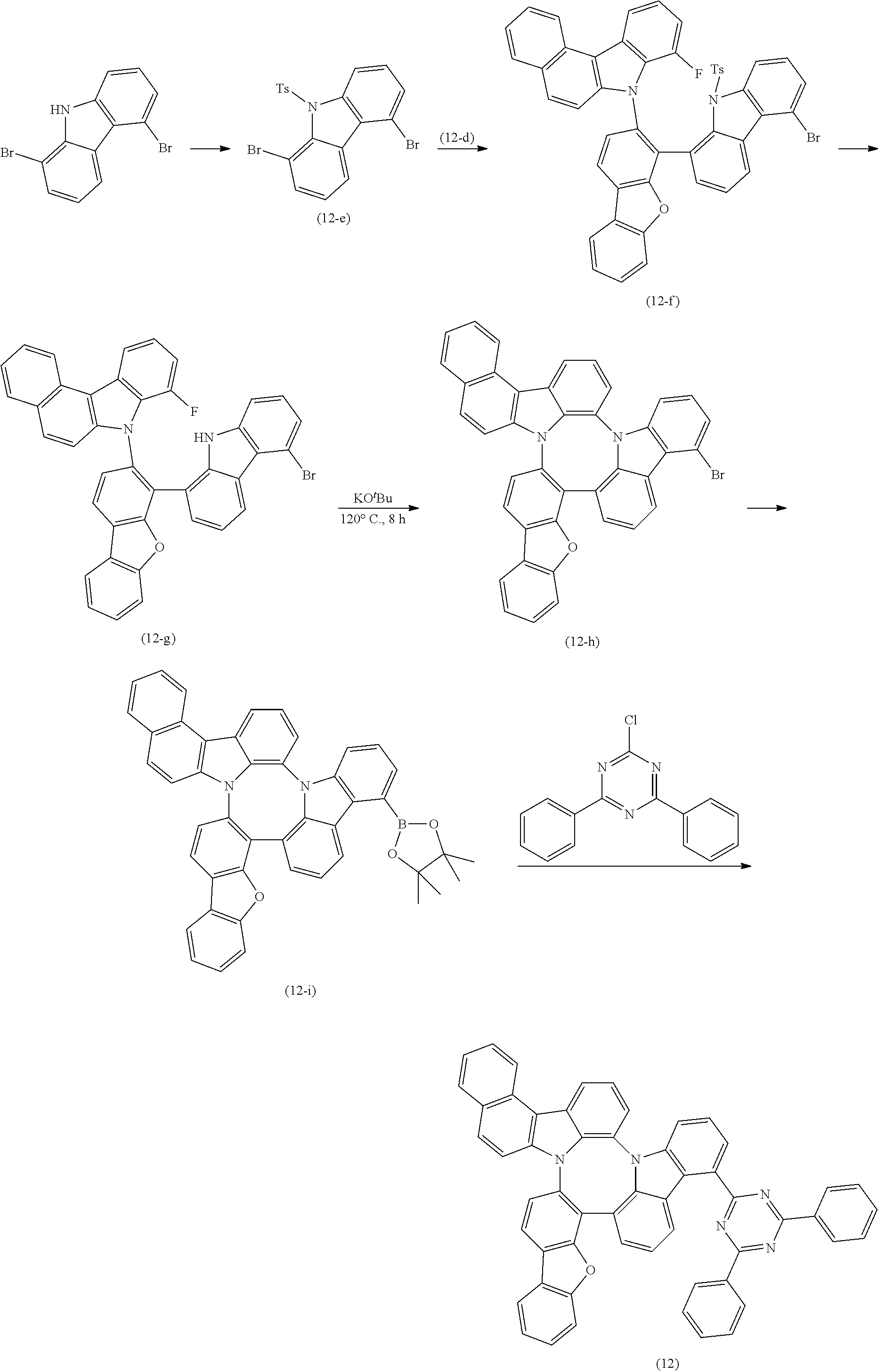

### Synthesis of Intermediate (12-a)

According to the synthesis method of the intermediate (1-d), the differences were in that 1-bromo-2-nitronaphthalene was substitute for the intermediate (1-b) and 3-fluorophenylboronic acid was substitute for the intermediate (1-c) to obtain a solid intermediate (12-a) with yield of 58%.

### Synthesis of Intermediate (12-b)

According to the synthesis method of the intermediate (2-b), the difference was in that the intermediate (12-a) was substitute for the intermediate (2-a) to obtain a solid intermediate (12-b) with yield of 62%.

### Synthesis of Intermediate (12-c)

According to the synthesis method of the intermediate (1-a), the differences were in that 3-fluoro-4-bromodibenzofuran was substitute for 2-bromofluorobenzene and the intermediate (12-b) was substitute for 1-fluorocarbazole to obtain a white solid intermediate (12-c) with yield of 49%.

### Synthesis of Intermediate (12-d)

According to the synthesis method of the intermediate (1-b), the difference was in that the intermediate (12-c) was substitute for the intermediate (1-a) to obtain a solid intermediate (12-d) with yield of 68%.

### Synthesis of Intermediate (12-e)

According to the synthesis method of the intermediate (1-c), the difference was in that 1,5-dibromocarbazole was substitute for 1,3-dibromocarbazole to obtain a solid intermediate (12-e) with yield of 85%.

### Synthesis of Intermediate (12-f)

According to the synthesis method of the intermediate (1-d), the differences were in that the intermediate (12-e) was substitute for the intermediate (1-b) and the intermediate (12-d) was substitute for the intermediate (1-c) to obtain a solid intermediate (12-f) with yield of 38%.

### Synthesis of Intermediate (12-g)

According to the synthesis method of the intermediate (1-e), the difference was in that the intermediate (12-f) was substitute for the intermediate (1-d) to obtain a solid intermediate (12-g) with yield of 88%.

### Synthesis of Intermediate (12-h)

According to the synthesis method of the intermediate (1-f), the difference was in that the intermediate (12-g) was substitute for the intermediate (1-e) to obtain a white solid intermediate (12-h) with yield of 59%.

### Synthesis of Intermediate (12-i)

According to the synthesis method of the intermediate (1-g), the difference was in that the intermediate (12-h) was substitute for the intermediate (1-f) to obtain a solid intermediate (12-i) with yield of 67%.

### Synthesis of Organic Compound (12)

According to the synthesis method of the compound (1), the difference was in that the intermediate (12-i) was substitute for the intermediate (1-g) to obtain a solid organic compound (12) with yield of 50%. Mass spectrometry peak: m/z=777.2530 [M]⁺.

### Synthesis Example 13: Synthesis of Organic Compound (13)

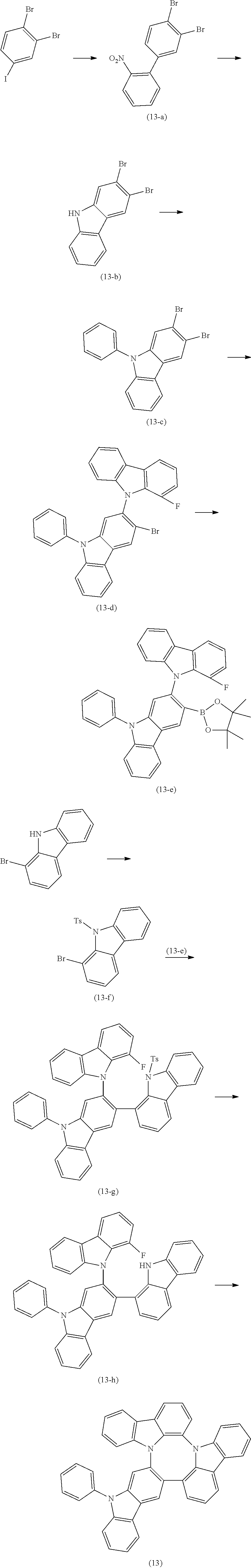

### Synthesis of Intermediate (13-a)

According to the synthesis method of the intermediate (1-d), the differences were in that 1,2-dibromo-4-iodobenzene was substitute for the intermediate (1-b) and 1-bromo-2-nitrobenzene was substitute for the intermediate (1-c) to obtain a solid intermediate (13-a) with yield of 70%.

### Synthesis of Intermediate (13-b)

According to the synthesis method of the intermediate (2-b), the difference was in that the intermediate (13-a) was substitute for the intermediate (2-a) to obtain a solid intermediate (13-b) with yield of 62%.

### Synthesis of Intermediate (13-c)

According to the synthesis method of the intermediate (1-a), the differences were in that fluorobenzene was substitute for 2-bromofluorobenzene and the intermediate (13-b) was substitute for 1-fluorocarbazole to obtain a white solid intermediate (13-c) with yield of 87%.

### Synthesis of Intermediate (13-d)

According to the synthesis method of the intermediate (1-d), the differences were in that 1-fluorocarbazole was substitute for the intermediate (1-b) and the intermediate (13-c) was substitute for the intermediate (1-c) to obtain a solid intermediate (13-d) with yield of 46%.

### Synthesis of Intermediate (13-e)

According to the synthesis method of the intermediate (1-b), the difference was in that the intermediate (13-d) was substitute for the intermediate (1-a) to obtain a solid intermediate (13-e) with yield of 73%.

### Synthesis of Intermediate (13-f)

According to the synthesis method of the intermediate (1-c), the difference was in that 1-bromocarbazole was substitute for 1,3-dibromocarbazole to obtain a solid intermediate (13-f) with yield of 84%.

### Synthesis of Intermediate (13-g)

According to the synthesis method of the intermediate (1-d), the differences were in that the intermediate (13-f) was substitute for the intermediate (1-b) and the intermediate (13-e) was substitute for the intermediate (1-c) to obtain a solid intermediate (13-g) with yield of 72%.

### Synthesis of Intermediate (13-h)

According to the synthesis method of the intermediate (1-e), the difference was in that the intermediate (13-g) was substitute for the intermediate (1-d) to obtain a solid intermediate (13-h) with yield of 91%.

### Synthesis of Organic Compound (13)

According to the synthesis method of the intermediate (1-f), the difference was in that the intermediate (13-h) was substitute for the intermediate (1-e) to obtain a white solid organic compound (13) with yield of 46% Mass spectrometry peak: m/z=571.2022 [M]⁺.

### Synthesis Example 14: Synthesis of Organic Compound (14)

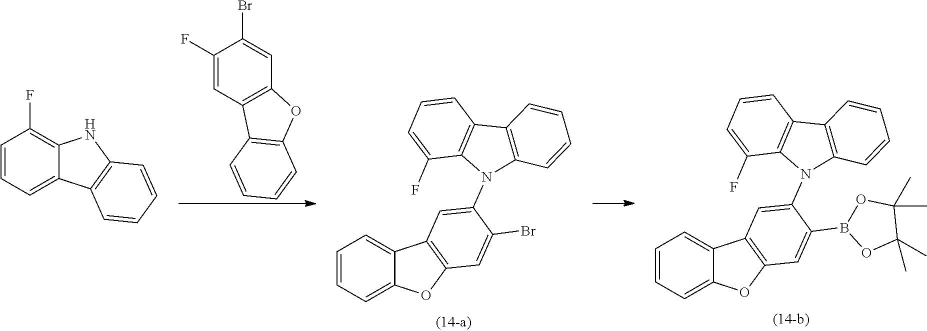 [SMILES: Fc1cccc2c1[nH]c1ccccc12 ; Fc1cc2c(cc1Br)oc1ccccc12 ; Fc1cccc2c3ccccc3n(-c3cc4c(cc3Br)oc3ccccc34)c12 ; CC1(C)OB(c2cc3oc4ccccc4c3cc2-n2c3ccccc3c3cccc(F)c32)OC1(C)C]

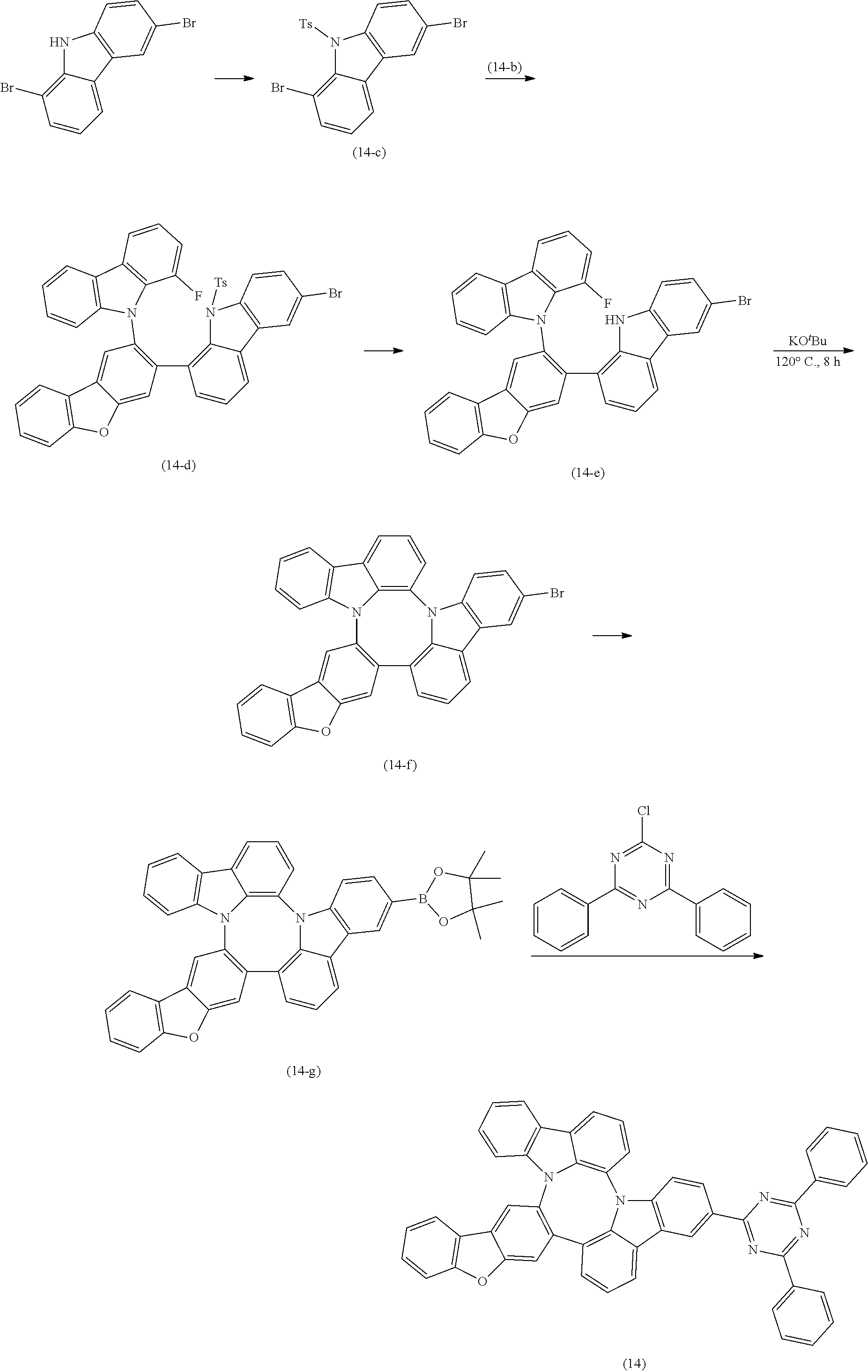

### Synthesis of Intermediate (14-a)

According to the synthesis method of the intermediate (1-a), the difference was in that 2-fluoro-3-bromodibenzofuran was substitute for 2-bromofluorobenzene to obtain a white solid intermediate (14-a) with yield of 53%.

### Synthesis of Intermediate (14-b)

According to the synthesis method of the intermediate (1-b), the difference was in that the intermediate (14-a) was substitute for the intermediate (1-a) to obtain a solid intermediate (14-b) with yield of 72%.

### Synthesis of Intermediate (14-c)

According to the synthesis method of the intermediate (1-c), the difference was in that 1,6-dibromocarbazole was substitute for 1,3-dibromocarbazole to obtain a solid intermediate (14-c) with yield of 80%.

### Synthesis of Intermediate (14-d)

According to the synthesis method of the intermediate (1-d), the differences were in that the intermediate (14-c) was substitute for the intermediate (1-b) and the intermediate (14-b) was substitute for the intermediate (1-c) to obtain a solid intermediate (14-d) with yield of 47%.

### Synthesis of Intermediate (14-e)

According to the synthesis method of the intermediate (1-e), the difference was in that the intermediate (14-d) was substitute for the intermediate (1-d) to obtain a solid intermediate (14-e) with yield of 88%.

### Synthesis of Intermediate (14-f)

According to the synthesis method of the intermediate (1-f), the difference was in that the intermediate (14-e) was substitute for the intermediate (1-e) to obtain a white solid intermediate (14-f) with yield of 73%.

### Synthesis of Intermediate (14-g)

According to the synthesis method of the intermediate (1-g), the difference was in that the intermediate (14-f) was substitute for the intermediate (1-f) to obtain a solid intermediate (14-g) with yield of 68%.

### Synthesis of Organic Compound (14)

According to the synthesis method of the compound (1), the difference was in that the intermediate (14-g) was substitute for the intermediate (1-g) to obtain a solid organic compound (14) with yield of 76%. Mass spectrometry peak: m/z=727.2418 [M]⁺.

### Synthesis Example 15: Synthesis of Organic Compound (15)

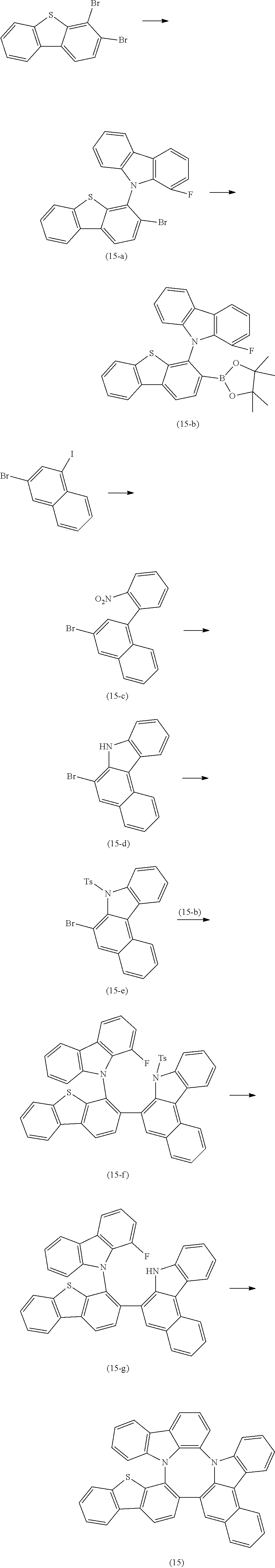

### Synthesis of Intermediate (15-a)

According to the synthesis method of the intermediate (1-d), the differences were in that 3,4-dibromobenzothiophene was substitute for the intermediate (1-b) and 1-fluorocarbazole was substitute for the intermediate (1-c) to obtain a solid intermediate (15-a) with yield of 66%.

### Synthesis of Intermediate (15-b)

According to the synthesis method of the intermediate (1-b), the difference was in that the intermediate (15-a) was substitute for the intermediate (1-a) to obtain a solid intermediate (15-b) with yield of 68%.

### Synthesis of Intermediate (15-c)

According to the synthesis method of the intermediate (1-d), the differences were in that 3-bromo-1-iodonaphthalene was substitute for the intermediate (1-b) and 2-bromophenylboronic acid was substitute for the intermediate (1-c) to obtain a solid intermediate (15-c) with yield of 57%.

### Synthesis of Intermediate (15-d)

According to the synthesis method of the intermediate (2-b), the difference was in that the intermediate (15-c) was substitute for the intermediate (2-a) to obtain a solid intermediate (15-d) with yield of 70%.

### Synthesis of Intermediate (15-e)

According to the synthesis method of the intermediate (1-c), the difference was in that the intermediate (15-d) was substitute for 1,3-dibromocarbazole to obtain a solid intermediate (15-e) with yield of 73%.

### Synthesis of Intermediate (15-f)

According to the synthesis method of the intermediate (1-d), the differences were in that the intermediate (15-e) was substitute for the intermediate (1-b) and the intermediate (15-b) was substitute for the intermediate (1-c) to obtain a solid intermediate (15-f) with yield of 65%.

### Synthesis of Intermediate (15-g)

According to the synthesis method of the intermediate (1-e), the difference was in that the intermediate (15-f) was substitute for the intermediate (1-d) to obtain a solid intermediate (15-g) with yield of 95%.

### Synthesis of Organic Compound (15)

According to the synthesis method of the intermediate (1-f), the difference was in that the intermediate (15-g) was substitute for the intermediate (1-e) to obtain a white solid organic compound (15) with yield of 52%. Mass spectrometry peak: m/z=562.1505 [M]⁺.

### Synthesis Example 16: Synthesis of Organic Compound (16)

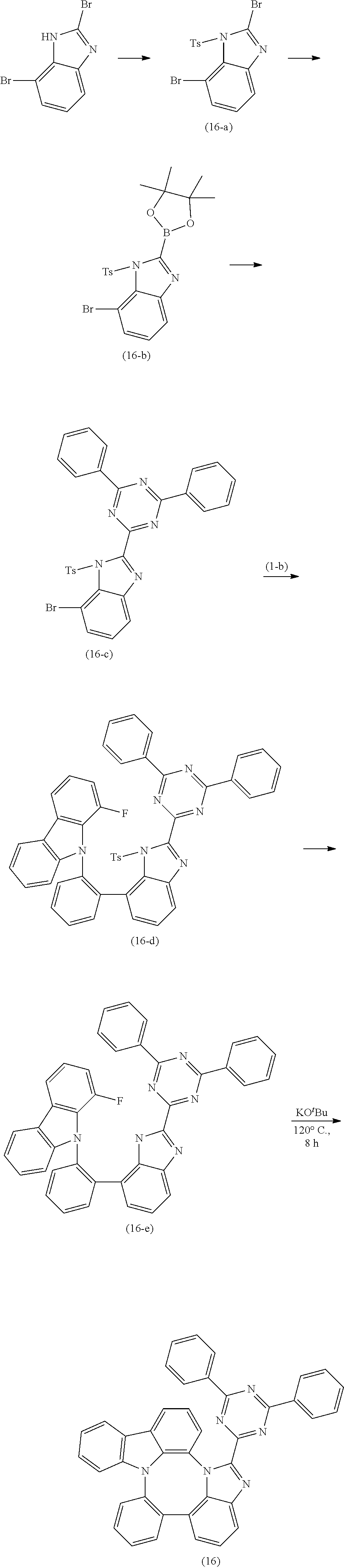

### Synthesis of Intermediate (16-a)

According to the synthesis method of the intermediate (1-c), the difference was in that 2,7-dibromobenzimidazole was substitute for 1,3-dibromocarbazole to obtain a solid intermediate (16-a) with yield of 84%.

### Synthesis of Intermediate (16-b)

According to the synthesis method of the intermediate (1-b), the difference was in that the intermediate (16-a) was substitute for the intermediate (1-a) to obtain a solid intermediate (16-b) with yield of 85%.

### Synthesis of Intermediate (16-c)

According to the synthesis method of the intermediate (1-d), the differences were in that the intermediate (16-b) was substitute for the intermediate (1-b) and 2-chloro-4,6-diphenyl-1,3,5-triazine was substitute for the intermediate (1-c) to obtain a white solid intermediate (16-c) with yield of 69%.

### Synthesis of Intermediate (16-d)

According to the synthesis method of the intermediate (1-d), the difference was in that the intermediate (16-c) was substitute for the intermediate (1-c) to obtain a solid intermediate (16-d) with yield of 71%.

### Synthesis of Intermediate (16-e)

According to the synthesis method of the intermediate (1-e), the difference was in that the intermediate (16-d) was substitute for the intermediate (1-d) to obtain a solid intermediate (16-e) with yield of 93%.

### Synthesis of Organic Compound (16)

According to the synthesis method of the intermediate (1-f), the difference was in that the intermediate (16-e) was substitute for the intermediate (1-e) to obtain a white solid organic compound (16) with yield of 66%. Mass spectrometry peak: m/z=588.2125 [M]⁺.

### Synthesis Example 17: Synthesis of Organic Compound (17)

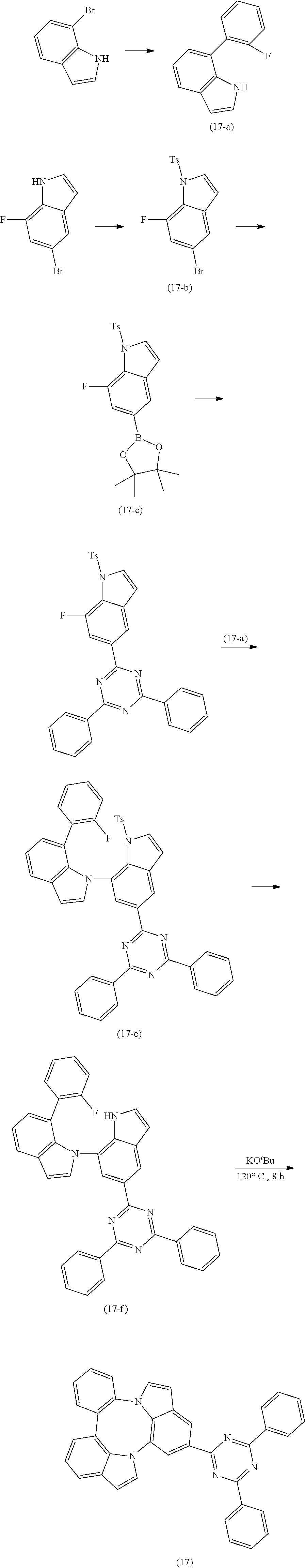

### Synthesis of Intermediate (17-a)

According to the synthesis method of the intermediate (1-d), the differences were in that 7-bromoindole was substitute for the intermediate (1-b) and 2-fluorophenylboronic acid was substitute for the intermediate (1-c) to obtain a solid intermediate (17-a) with yield of 80%.

### Synthesis of Intermediate (17-b)

According to the synthesis method of the intermediate (1-c), the difference was in that 5-bromo-7-fluoroindole was substitute for 1,3-dibromocarbazole to obtain a solid intermediate (17-b) with yield of 78%.

### Synthesis of Intermediate (17-c)

According to the synthesis method of the intermediate (1-b), the difference was in that the intermediate (17-b) was substitute for the intermediate (1-a) to obtain a solid intermediate (17-c) with yield of 65%.

### Synthesis of Intermediate (17-d)

According to the synthesis method of the intermediate (1-d), the differences were in that the intermediate (17-c) was substitute for the intermediate (1-b) and 2-chloro-4,6-diphenyl-1,3,5-triazine was substitute for the intermediate (1-c) to obtain a white solid intermediate (17-d) with yield of 64%.

### Synthesis of Intermediate (17-e)

According to the synthesis method of the intermediate (1-d), the differences were in that the intermediate (17-d) was substitute for the intermediate (1-b) and the intermediate (17-a) was substitute for the intermediate (1-c) to obtain a solid intermediate (17-e) with yield of 61%.

### Synthesis of Intermediate (17-f)

According to the synthesis method of the intermediate (1-e), the difference was in that the intermediate (17-e) was substitute for the intermediate (1-d) to obtain a solid intermediate (17-f) with yield of 95%.

### Synthesis of Organic Compound (17)

According to the synthesis method of the intermediate (1-f), the difference was in that the intermediate (17-f) was substitute for the intermediate (1-e) to obtain a white solid organic compound (17) with yield of 58%. Mass spectrometry peak: m/z=537.2046 [M]⁺.

### Synthesis Example 18: Synthesis of Organic Compound (18)

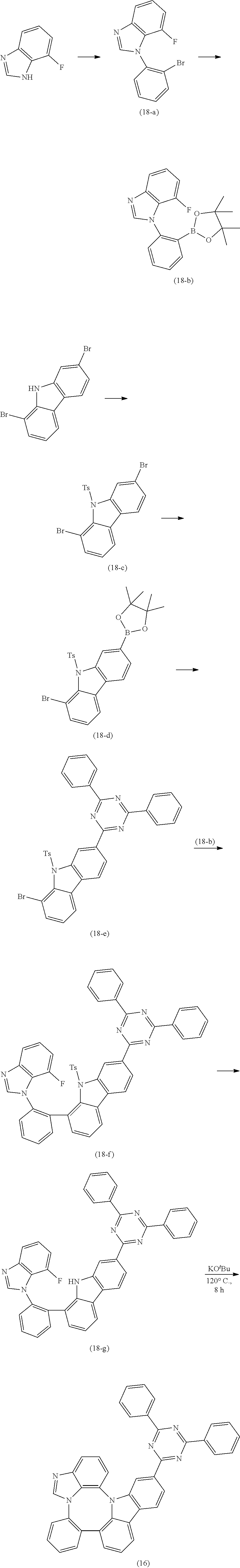

### Synthesis of Intermediate (18-a)

According to the synthesis method of the intermediate (1-d), the differences were in that 7-fluorobenzimidazole was substitute for the intermediate (1-b) and 2-bromofluorobenzene was substitute for the intermediate (1-c) to obtain a solid intermediate (18-a) with yield of 52%.

### Synthesis of Intermediate (18-b)

According to the synthesis method of the intermediate (1-b), the difference was in that the intermediate (18-a) was substitute for the intermediate (1-a) to obtain a solid intermediate (18-b) with yield of 72%.

### Synthesis of Intermediate (18-c)

According to the synthesis method of the intermediate (1-c), the difference was in that 1,7-dibromocarbazole was substitute for 1,3-dibromocarbazole to obtain a solid intermediate (18-c) with yield of 61%.

### Synthesis of Intermediate (18-d)

According to the synthesis method of the intermediate (1-b), the difference was in that the intermediate (18-c) was substitute for the intermediate (1-a) to obtain a solid intermediate (18-d) with yield of 76%.

### Synthesis of Intermediate (18-e)

According to the synthesis method of the intermediate (1-d), the differences were in that the intermediate (18-d) was substitute for the intermediate (1-b) and 2-chloro-4,6-diphenyl-1,3,5-triazine was substitute for the intermediate (1-c) to obtain a white solid intermediate (18-e) with yield of 61%.

### Synthesis of Intermediate (18-f)

According to the synthesis method of the intermediate (1-d), the differences were in that the intermediate (18-e) was substitute for the intermediate (1-b) and the intermediate (18-b) was substitute for intermediate (1-c) to obtain a white solid intermediate (18-f) with yield of 54%.

### Synthesis of Intermediate (18-g)

According to the synthesis method of the intermediate (1-e), the difference was in that the intermediate (18-f) was substitute for the intermediate (1-d) to obtain a solid intermediate (18-g) with yield of 90%.

### Synthesis of Organic Compound (18)

According to the synthesis method of the intermediate (1-f), the difference was in that the intermediate (18-g) was substitute for the intermediate (1-e) to obtain a white solid organic compound (18) with yield of 44%. Mass spectrometry peak: m/z=588.2125 [M]⁺.

### Synthesis Example 19: Synthesis of Organic Compound (19)

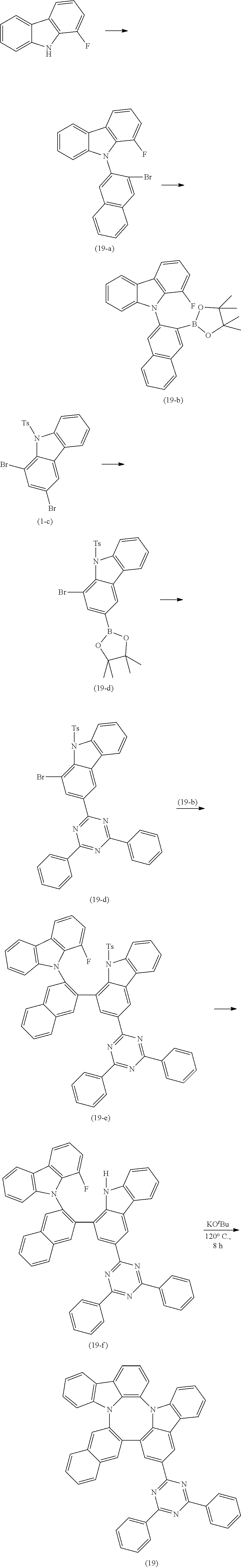

### Synthesis of Intermediate (19-a)

According to the synthesis method of the intermediate (18-a), the differences were in that 1-fluorocarbazole was substitute for 7-fluorobenzimidazole and 2-bromo-3-fluoronaphthalene was substitute for 2-bromofluorobenzene to obtain a solid intermediate (19-a) with yield of 57%.

### Synthesis of Intermediate (19-b)

According to the synthesis method of the intermediate (1-b), the difference was in that the intermediate (19-a) was substitute for the intermediate (1-a) to obtain a solid intermediate (19-b) with yield of 65%.

### Synthesis of Intermediate (19-c)

According to the synthesis method of the intermediate (1-b), the difference was in that the intermediate (1-c) was substitute for the intermediate (1-a) to obtain a solid intermediate (19-c) with yield of 68%.

### Synthesis of Intermediate (19-d)

According to the synthesis method of the intermediate (1-d), the differences were in that the intermediate (19-c) was substitute for the intermediate (1-b) and 2-chloro-4,6-diphenyl-1,3,5-triazine was substitute for the intermediate (1-c) to obtain a white solid intermediate (19-d) with yield of 66%.

### Synthesis of Intermediate (19-e)

According to the synthesis method of the intermediate (1-d), the differences were in that the intermediate (19-d) was substitute for the intermediate (1-b) and the intermediate (19-b) was substitute for the intermediate (1-c) to obtain a white solid intermediate (19-e) with yield of 50%.

### Synthesis of Intermediate (19-f)

According to the synthesis method of the intermediate (1-e), the difference was in that the intermediate (19-e) was substitute for the intermediate (1-d) to obtain a solid intermediate (19-f) with yield of 87%.

### Synthesis of Organic Compound (19)

According to the synthesis method of the intermediate (1-f), the difference was in that the intermediate (19-f) was substitute for the intermediate (1-e) to obtain a white solid organic compound (19) with yield of 41%. Mass spectrometry peak: m/z=687.8145 [M]⁺.

### 2. Organic Compounds and Energy Levels Thereof

Energy levels of the organic compounds were obtained by quantum calculation, such as Gaussian 03W (Gaussian Inc.) using time-dependent density functional theory (TD-DFT), specific simulation methods can refer to patent application WO 2011/41110. Molecular geometry was first optimized using a semi empirical method “Ground State/Hartree-Fock/Default Spin/LanL2MB” (Charge0/Spin Singlet). Then energy levels of the organic compounds were calculated by the TD-DFT method obtain “TD-SCF/DFT/Default Spin/B3PW91/gen geo=connectivity pseudo=LanL2” (Charge 0/Spin Singlet). HOMO and LUMO energy levels were calculated according to the following calibration equations, and S1 and T1 energy levels were directly used.

$$\begin{matrix}HOMO ( eV ) = ( ( HOMO ( Gaussian ) \times 27. 212 ) - 0.98 \; 99 ) / 1.1206 ; \\ LUMO ( eV ) = ( ( LUMO ( Gaussian ) \times 27. 212 ) - 2.0 \; 041 ) / 1.385 ;\end{matrix}$$

in which HOMO (G) and LUMO (G) were the direct calculation results of Gaussian 03W, in Hartree. The results are shown in Table 1:

TABLE 1

| Material | HOMO [eV] | LUMO [eV] | T1 [eV] | S1 [eV] | ΔE_{S-T} [eV] |
|---|---|---|---|---|---|
| (1) | −5.78 | −2.91 | 2.68 | 3.06 | 0.38 |
| (2) | −5.55 | −2.2 | 2.74 | 3.04 | 0.26 |
| (3) | −5.49 | −2.63 | 2.80 | 3.14 | 0.34 |
| (4) | −5.64 | −2.77 | 2.60 | 3.11 | 0.48 |
| (5) | −5.74 | −2.89 | 2.75 | 3.03 | 0.21 |
| (6) | −5.54 | −2.68 | 2.77 | 2.98 | 0.21 |
| (7) | −5.66 | −2.87 | 2.74 | 2.99 | 0.25 |
| (8) | −5.70 | −2.71 | 2.68 | 3.01 | 0.33 |
| (9) | −5.43 | −2.65 | 2.70 | 3.13 | 0.43 |
| (10) | −5.62 | −2.74 | 2.74 | 3.08 | 0.34 |
| (11) | −5.44 | −2.99 | 2.69 | 3.14 | 0.45 |
| (12) | −5.64 | −3.01 | 2.83 | 3.04 | 0.21 |
| (13) | −5.78 | −2.83 | 2.74 | 2.99 | 0.25 |
| (14) | −5.98 | −2.59 | 2.83 | 3.20 | 0.37 |
| (15) | −5.84 | −2.74 | 2.76 | 3.15 | 0.39 |
| (16) | −5.56 | −2.89 | 2.89 | 3.09 | 0.2 |
| (17) | −5.43 | −2.94 | 2.67 | 3.07 | 0.4 |
| (18) | −5.88 | −2.52 | 2.74 | 3.01 | 0.27 |
| (19) | −5.68 | −2.43 | 2.70 | 3.02 | 0.32 |
| (Ref) | −6.03 | −2.86 | 2.60 | 2.98 | 0.38 |

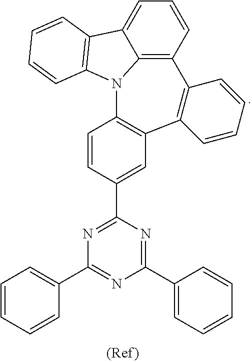 [SMILES: c1ccc(-c2nc(-c3ccccc3)nc(-c3ccc4c(c3)-c3ccccc3-c3cccc5c6ccccc6n-4c35)n2)cc1]

### 3. Preparation and Characterization of OLED Devices

The structures of OLED devices are as follows:
ITO/HIL (100 Å)/HTL (450 Å)/EBM (50 Å)/EML (400 Å)/ETL (350 Å)/cathode;
in which EML is composed of H-Host, E-Host, and transition metal complex of Ir(ppy)₃. A mass ratio of H-Host to E-Host is 6:4, and a doping amount of Ir(ppy)₃ is 10% w/w of the total mass of H-Host and E-Host. E-Host is selected from the organic compounds (1) to (18) in examples of the disclosure or (Ref). ETL is composed of 8-hydroxyquinoline lithium (LiQ) doped with 40% w/w of ETM. The structures of materials used in the devices are as follows:

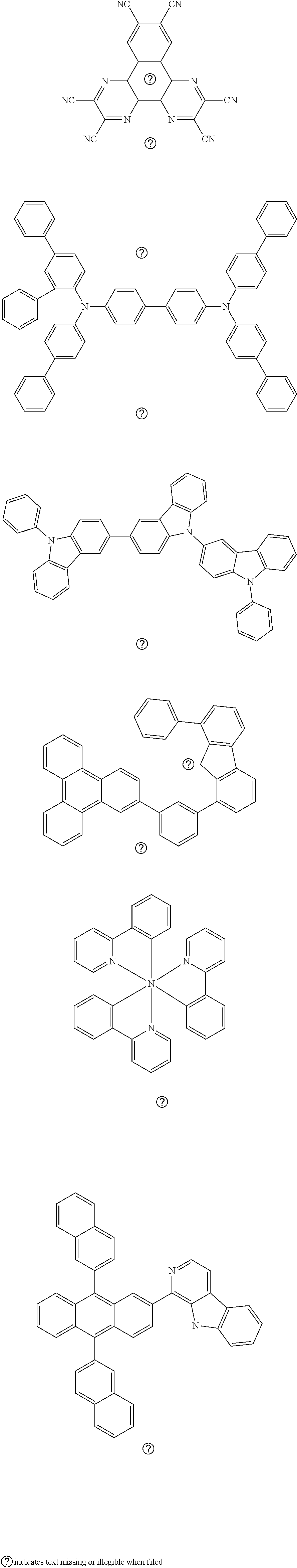

The preparation steps for the OLED devices are as follows:
a. cleaning a conductive glass substrate. When used for the first time, the conductive glass substrate was cleaned using various solvents, such as chloroform, ketone, and isopropanol, followed by UV ozone plasma treatment;
b. forming the HIL (100 Å), the HTM (450 Å), the EBM (50 Å), the EML (400 Å), and the ETL (350 Å) by hot vapor deposition in high vacuum (10⁻⁶ millibars);
c. forming the cathode consisting of a LiF layer (1 nm) and an Al layer (150 nm) by hot evaporation in high vacuum (10⁻⁶ millibars); and
d. encapsulation. The devices were encapsulated by UV cured resin in a nitrogen glove box.

The current-voltage-brightness (JVL) characteristics of the OLED devices were measured by characterization devices, to obtain parameters such as efficiency, external quantum efficiency, and lifetime of the devices. Compared with the classical triazine molecule (Ref) with a seven-membered ring, relative parameters of the OLED devices are shown in Table 2:

TABLE 2

| | | | | External | |
|---|---|---|---|---|---|
| | | Starting | | Quantum | Lifetime |
| Device | E-host | Voltage | Efficiency | Efficiency | (T₉₅, h) @ |
| Example | Material | (V) | (cd/A) | (EQE) | 50 mA/cm² |
| OLED-1 | (1) | 95% | 108% | 114% | 113% |
| OLED-2 | (2) | 93% | 115% | 113% | 114% |
| OLED-3 | (3) | 92% | 119% | 117% | 115% |
| OLED-4 | (4) | 93% | 111% | 113% | 112% |
| OLED-5 | (5) | 93% | 110% | 112% | 111% |
| OLED-6 | (6) | 91% | 114% | 109% | 116% |
| OLED-7 | (7) | 92% | 104% | 106% | 108% |
| OLED-8 | (8) | 97% | 105% | 105% | 107% |
| OLED-9 | (9) | 94% | 106% | 108% | 113% |
| OLED-10 | (10) | 92% | 109% | 109% | 112% |
| OLED-11 | (11) | 93% | 113% | 116% | 117% |
| OLED-12 | (12) | 95% | 109% | 108% | 115% |
| OLED-13 | (13) | 95% | 108% | 106% | 112% |
| OLED-14 | (14) | 96% | 110% | 104% | 114% |
| OLED-15 | (15) | 95% | 111% | 103% | 114% |
| OLED-16 | (16) | 97% | 106% | 105% | 105% |
| OLED-17 | (17) | 99% | 102% | 101% | 104% |
| OLED-18 | (18) | 97% | 105% | 107% | 107% |
| OLED-19 | (19) | 95% | 109% | 108% | 110% |
| OLED-20 | (Ref) | 100% | 100% | 100% | 100% |

It can be seen from the data in Table 2 that the disclosure can significantly improve the external quantum efficiency and lifetime of OLED devices by using the nitrogen-containing heterocyclic compounds as E-host materials for light-emitting layers (EMLs).

Not bondary to the theory, this is because the eight-membered ring in the organic compound of the disclosure is relatively stable, and the organic compound has a larger ring structure, which can increase the conjugation system of the organic compound and make the organic compound more stable, thus improving stability of devices and reducing the starting voltages to increase lifetime of devices.

Further, by optimizing the combination of HTM, ETM, and host materials, performances of the devices, especially efficiency, driving voltage, and lifetime, can be further improved.

Various technical features of the above examples can be optionally combined. To make the description concise, the above examples do not illustrate all possible combinations of various technical features. However, as long as there is no contradiction in the combinations of these technical features, they can be considered within the scope of this specification.

The above examples only illustrate several examples of the disclosure, and the description to these examples is specific and detailed, but cannot be understood as a limitation on the scope of the disclosure. It should be pointed out that for ordinary skilled in the art, several modifications and improvements can be made without departing from the concept of this disclosure, all of which fall within the scope of the disclosure. Therefore, the scope of this disclosure shall be based on the attached claims.

1. A nitrogen-containing heterocyclic compound having a structure represented by formula (1): 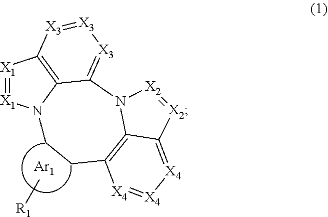
wherein,
Ar₁ is selected from a substituted or unsubstituted aromatic group having 6 to 40 ring atoms or a substituted or unsubstituted heteroaromatic group having 5 to 40 ring atoms;
X₁ at each occurrence is independently selected from CR₂ or N;
X₂ at each occurrence is independently selected from CR₃ or N;
X₃ at each occurrence is independently selected from CR₄ or N;
X₄ at each occurrence is independently selected from CR₅ or N;
R₁ to R₅ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
R₁ and X₄ are connected to form a ring or not form a ring together; two adjacent R₂ groups are connected to form a ring or not form a ring together; two adjacent R₃ groups are connected to form a ring or not form a ring together; two adjacent R₄ groups are connected to form a ring or not form a ring together; and two adjacent R₅ groups are connected to form a ring or not form a ring together.

2. The nitrogen-containing heterocyclic compound of claim 1, wherein the nitrogen-containing heterocyclic compound has a structure represented by any one of formulae (2-1) to (2-3): 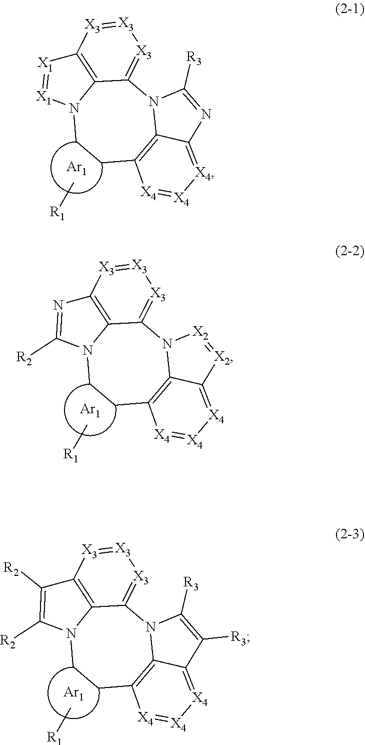
wherein two adjacent X₁ groups in formula (2-1) do not form a ring together, and two adjacent X₂ groups in formula (2-2) do not form a ring together.

3. The nitrogen-containing heterocyclic compound of claim 1, wherein the nitrogen-containing heterocyclic compound has a structure represented by any one of formulae (2-4) to (2-5): 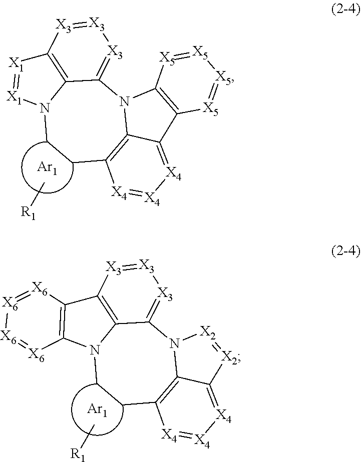
wherein two adjacent X₁ groups in formula (2-4) do not form a ring together; and two adjacent X₂ groups in formula (2-5) do not form a ring together;
X₅ at each occurrence is independently selected from CR₆ or N;
X₆ at each occurrence is independently selected from CR₇ or N; and
R₆ and R₇ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; two adjacent R₆ groups are connected to form a ring or not form a ring together; and two adjacent R₇ groups are connected to form a ring or not form a ring together.

4. The nitrogen-containing heterocyclic compound of claim 1, wherein the nitrogen-containing heterocyclic compound has a structure represented by formula (2-6): 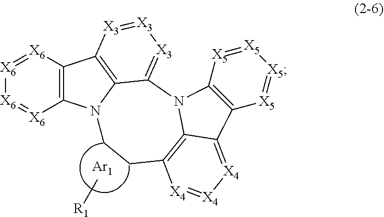
wherein,
X₅ at each occurrence is independently selected from CR₆ or N;
X₆ at each occurrence is independently selected from CR₇ or N; and
R₆ and R₇ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; two adjacent R₆ groups are connected to form a ring or not form a ring together; and two adjacent R₇ groups are connected to form a ring or not form a ring together.

5. The nitrogen-containing heterocyclic compound of claim 1, wherein Ar₁ is any one selected from a group consisting of following groups: 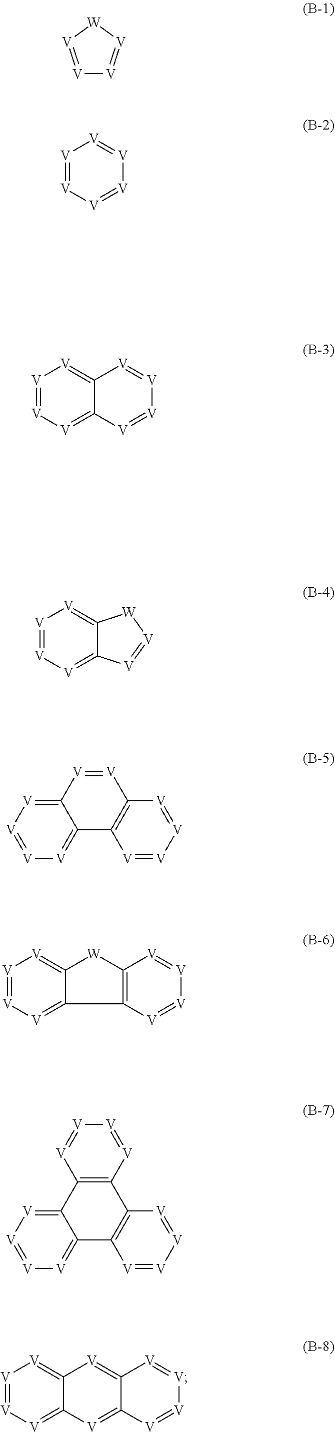
wherein,
V at each occurrence is independently selected from CR₁ or N, and at least one V is selected from CR₁;
W at each occurrence is independently selected from NR₈, PR₈, CR₈R₉, SiR₈R₉, O, S, S(═O)₂, or S(═O); and
R₈ and R₉ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups.

6. The nitrogen-containing heterocyclic compound of claim 5, wherein Ar₁ is any one selected from a group consisting of following groups: 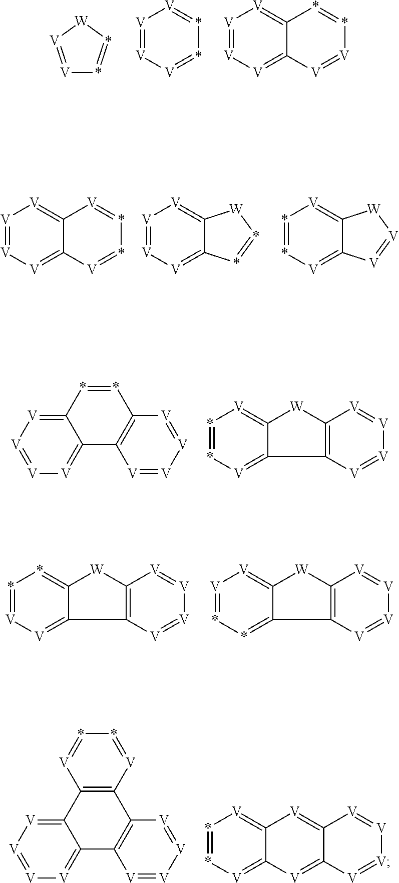
wherein * indicates a linking site.

7. The nitrogen-containing heterocyclic compound of claim 1, wherein the nitrogen-containing heterocyclic compound has a structure represented by any one of formulae (3-1) to (3-16): 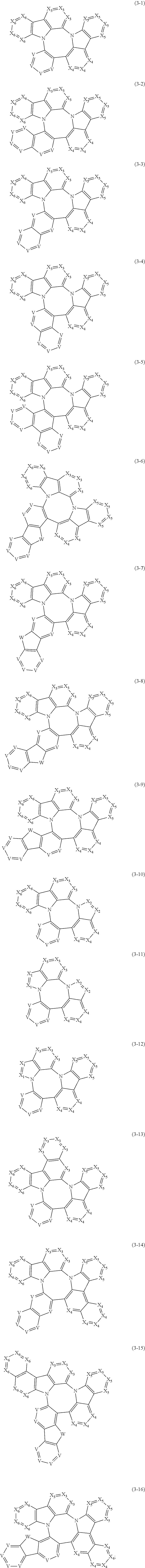
wherein X₅ at each occurrence is independently selected from CR₆ or N;
X₆ at each occurrence is independently selected from CR₇ or N;
V at each occurrence is independently selected from CR₁ or N, and at least one V is selected from CR₁;
W at each occurrence is independently selected from NR₈, PR₈, CR₈R₉, SiR₈R₉, O, S, S(═O)₂, or S(═O);
R₆ to R₉ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 5 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; two adjacent R₆ groups are connected to form a ring or not form a ring together; and two adjacent R₇ groups are connected to form a ring or not form a ring together; and
two adjacent X₂ groups in formula (3-10) do not form a ring together; and two adjacent X₁ groups in formulae (3-11) and (3-12) do not form a ring together.

8. The nitrogen-containing heterocyclic compound of claim 1, wherein the nitrogen-containing heterocyclic compound has a structure represented by any one of formulae (4-1) and (4-2): 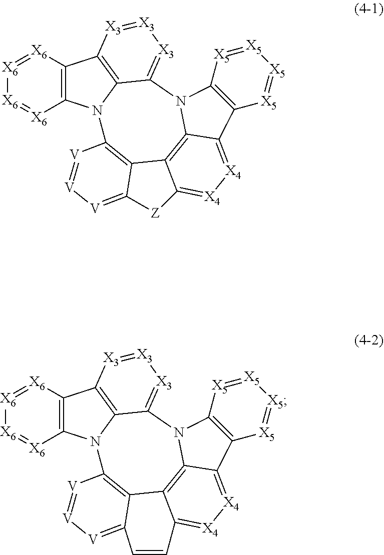
wherein,
Z at each occurrence is independently selected from NR₁₀, PR₁₀, CR₁₁R₁₂, SiR₁₁R₁₂, O, S, S(═O)₂, or S(═O);
R₁₀ to R₁₂ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
R₁₁ and R₁₂ are connected to form a ring or not form a ring together.

9. The nitrogen-containing heterocyclic compound of claim 1, wherein the nitrogen-containing heterocyclic compound comprises at least one group represented by formula (5): 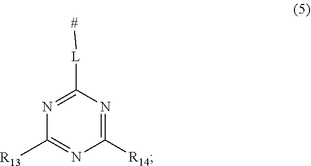
wherein,
L is selected from a single bond, a substituted or unsubstituted aromatic group having 6 to 40 ring atoms, or a substituted or unsubstituted heteroaromatic group having 5 to 40 ring atoms;
R₁₃ and R₁₄ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
# indicates a linking site.

10. The nitrogen-containing heterocyclic compound of claim 9, wherein R₁ to R₅ at each occurrence are independently from —H, -D, or a triazine group represented by formula (5).

11. The nitrogen-containing heterocyclic compound of claim 9, wherein the nitrogen-containing heterocyclic compound has a structure represented by any one of following structures:

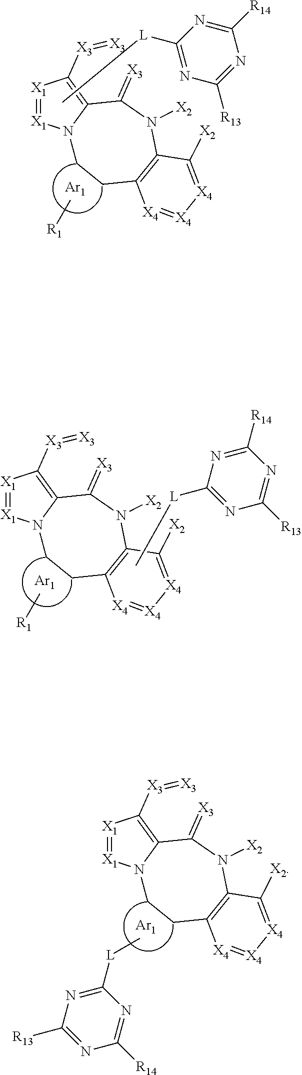

12. The nitrogen-containing heterocyclic compound of claim 9, wherein R₁₃ and R₁₄ at each occurrence are independently selected from a group consisting of following groups: 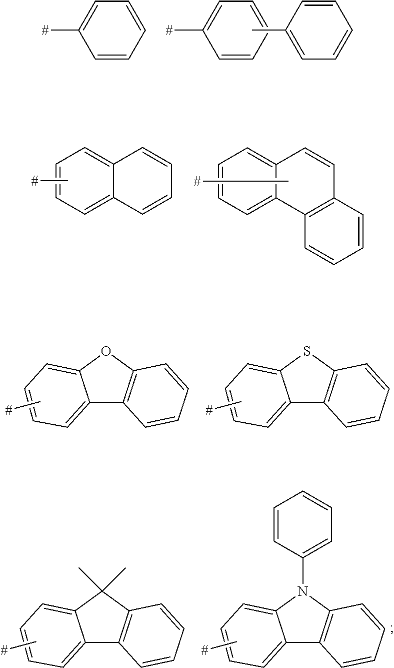
wherein # indicates a linking site.

13. A mixture, wherein the mixture comprises a nitrogen-containing heterocyclic compound as claimed in claim 1, and at least one organic functional material, wherein the organic functional material is a hole injection material, a hole transport material, an electron transport material, an electron injection material, an electron blocking material, a hole blocking material, a light-emitting material, a host material, or an organic dye.

14. A composition, wherein the composition comprises a nitrogen-containing heterocyclic compound and at least one organic solvent, wherein the nitrogen-containing heterocyclic compound has a structure represented by formula (1): 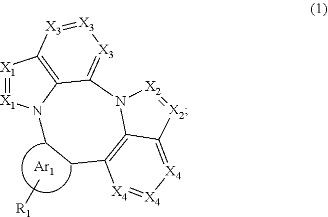
wherein Ar₁ is selected from a substituted or unsubstituted aromatic group having 6 to 40 ring atoms or a substituted or unsubstituted heteroaromatic group having 5 to 40 ring atoms;
X₁ at each occurrence is independently selected from CR₂ or N;
X₂ at each occurrence is independently selected from CR₃ or N;
X₃ at each occurrence is independently selected from CR₄ or N;
X₄ at each occurrence is independently selected from CR₅ or N;
R₁ to R₅ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
R₁ and X₄ are connected to form a ring or not form a ring together; two adjacent R₂ groups are connected to form a ring or not form a ring together; two adjacent R₃ groups are connected to form a ring or not form a ring together; two adjacent R₄ groups are connected to form a ring or not form a ring together; and two adjacent R₅ groups are connected to form a ring or not form a ring together.

15. An organic electronic device, wherein the organic electronic device comprises at least one functional layer, wherein a material of the functional layer comprises a nitrogen-containing heterocyclic compound having a structure represented by formula (1): 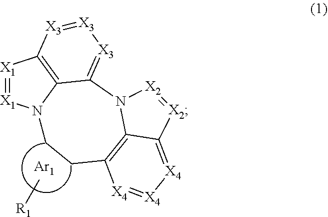
wherein Ar₁ is selected from a substituted or unsubstituted aromatic group having 6 to 40 ring atoms or a substituted or unsubstituted heteroaromatic group having 5 to 40 ring atoms;
X₁ at each occurrence is independently selected from CR₂ or N;
X₂ at each occurrence is independently selected from CR₃ or N;
X₃ at each occurrence is independently selected from CR₄ or N;
X₄ at each occurrence is independently selected from CR₅ or N;
R₁ to R₅ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
R₁ and X₄ are connected to form a ring or not form a ring together; two adjacent R₂ groups are connected to form a ring or not form a ring together; two adjacent R₃ groups are connected to form a ring or not form a ring together; two adjacent R₄ groups are connected to form a ring or not form a ring together; and two adjacent R₅ groups are connected to form a ring or not form a ring together.

16. The organic electronic device of claim 15, wherein the functional layer is a light-emitting layer.

17. The nitrogen-containing heterocyclic compound of claim 1, wherein the nitrogen-containing heterocyclic compound has a structure represented by any one of formulae (A-1) to (A-3): 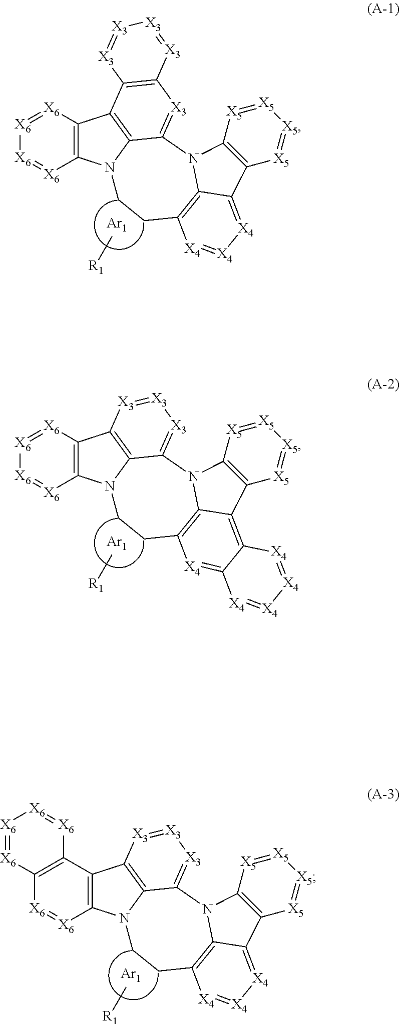
wherein X₅ at each occurrence is independently selected from CR₆ or N;
X₆ at each occurrence is independently selected from CR₇ or N;
R₆ and R₇ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
two adjacent R₆ groups are connected to form a ring or not form a ring together; and two adjacent R₇ groups are connected to form a ring or not form a ring together.

18. The nitrogen-containing heterocyclic compound of claim 1, wherein the nitrogen-containing heterocyclic compound has a structure represented by any one of formulae (I-1) to (I-7): 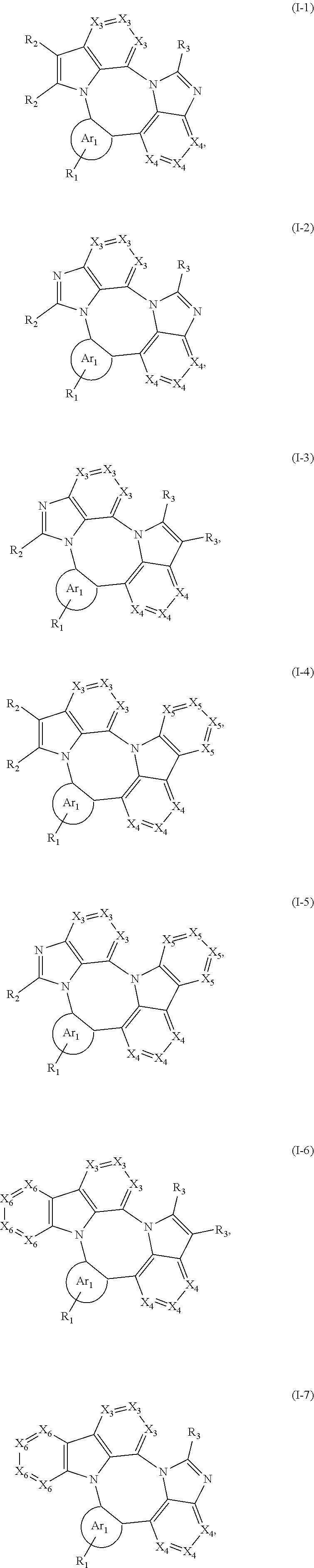
wherein two adjacent X₂ groups in formula (I-1) do not form a ring together, and two adjacent X₃ groups in formula (I-3) do not form a ring together;
X₅ at each occurrence is independently selected from CR₆ or N;
X₆ at each occurrence is independently selected from CR₇ or N; and
R₆ and R₇ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
two adjacent R₆ groups are connected to form a ring or not form a ring together; and two adjacent R₇ groups are connected to form a ring or not form a ring together.

19. The nitrogen-containing heterocyclic compound of claim 1, wherein the nitrogen-containing heterocyclic compound has a structure represented by any one of formulae (2-1) to (2-6): 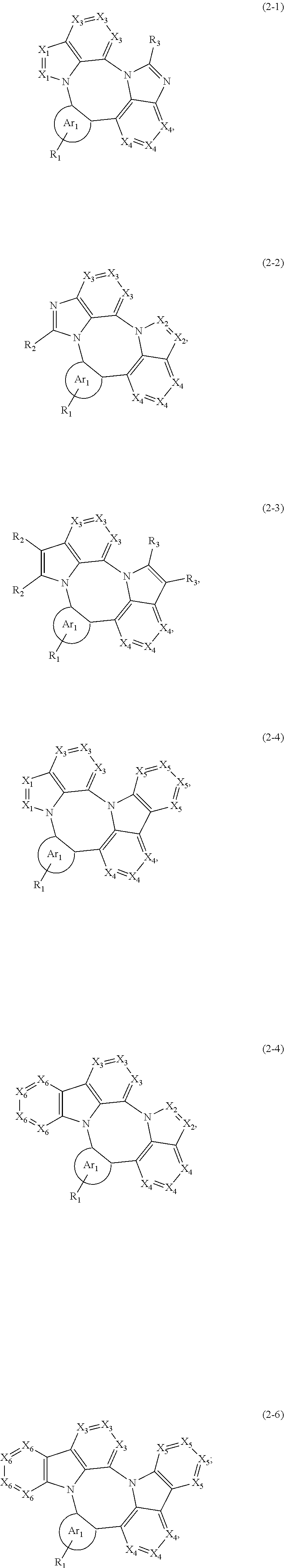
wherein two adjacent X₁ groups in formulae (2-1) and (2-4) do not form a ring together; and two adjacent X₂ groups in formulae (2-2) and (2-5) do not form a ring together;
X₅ at each occurrence is independently selected from CR₆ or N;
X₆ at each occurrence is independently selected from CR₇ or N;
R₆ and R₇ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups; and
two adjacent R₆ groups are connected to form a ring or not form a ring together; and two adjacent R₇ groups are connected to form a ring or not form a ring together.

20. The nitrogen-containing heterocyclic compound of claim 19, wherein R₁ to R₇ at each occurrence are independently from —H, -D, or a triazine group represented by formula (5): 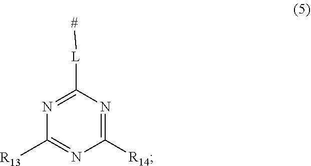
wherein L is selected from a single bond, a substituted or unsubstituted aromatic group having 6 to 40 ring atoms, or a substituted or unsubstituted heteroaromatic group having 5 to 40 ring atoms; and
R₁₃ and R₁₄ at each occurrence are independently selected from —H, -D, a linear alkyl group having 1 to 20 carbon atoms, an alkoxy group having 1 to 20 carbon atoms, a branched alkyl group having 3 to 20 carbon atoms, a cyclic alkyl group having 3 to 20 carbon atoms, a silyl group, a ketone group having 1 to 20 carbon atoms, a cyano group, an isocyano group, an isocyanate group, a hydroxyl group, a nitro group, an alkenyl group, —CF₃, —OCF₃, —Cl, —Br, —F, a substituted or unsubstituted aromatic group having 6 to 40 ring atoms, a substituted or unsubstituted heteroaromatic group having 5 to 40 ring atoms, a substituted or unsubstituted aryloxy group having 5 to 40 ring atoms, a substituted or unsubstituted heteroaryloxy group having 5 to 40 ring atoms, or any combination of these groups.
